SEQUENCE LISTING (12 sequences)

SEQ ID NO: 1 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 2 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 3 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 4 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 5 - protein, 107 aa, unidentified
  source 1..107: note = mouse
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTK

SEQ ID NO: 6 - protein, 110 aa, unidentified
  source 1..110: note = mouse
APDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPR

SEQ ID NO: 7 - protein, 110 aa, unidentified
  source 1..110: note = mouse
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 8 - protein, 110 aa, unidentified
  source 1..110: note = mouse
APNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIK

SEQ ID NO: 9 - protein, 110 aa, unidentified
  source 1..110: note = mouse
AGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPK

SEQ ID NO: 10 - protein, 263 aa, synthetic construct
  REGION 1..263: note = UCHT1
VQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGSGGGSGGGSGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGGSLPETGGHHHHHH

SEQ ID NO: 11 - protein, 155 aa, synthetic construct
  REGION 1..155: note = IL-2
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLTGGGGSGGGGSLPETGGHHHHHH

SEQ ID NO: 12 - protein, 229 aa, synthetic construct
  REGION 1..229: note = LC-GGGGY
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGY

(19) United States
(12) Patent Application Publication
(10) Pub. No.: US 2023/0405143 A1
(43) Pub. Date: Dec. 21, 2023

(54) TYROSINE-BASED ANTIBODY CONJUGATES

(71) Applicant: Synaffix B.V., Oss (NL)

(72) Inventors: Floris Louis VAN DELFT, Nijmegen (NL); Jorick Julian BRUINS, Neckargemund (DE); Hendrik Bauke ALBADA, Oosterbeek (NL); Maria Antonia WIJDEVEN, Oss (NL)

(21) Appl. No.: 18/320,403

(22) Filed: May 19, 2023

Related U.S. Application Data
(63) Continuation of application No. PCT/NL2021/050714, filed on Nov. 22, 2021.

(30) Foreign Application Priority Data
Nov. 20, 2020 (NL) 2026947

(51) Int. Cl. A61K 47/68; A61K 47/60

(52) U.S. Cl. CPC A61K 47/6889; A61K 47/68031; A61K 47/6813; A61K 47/6849; A61K 47/60

(57) ABSTRACT

A process for preparing a glycoprotein-conjugate is provided, comprising: (a) providing an N-glycoprotein having an exposed tyrosine residue, wherein the exposed tyrosine residue is located within 10 amino acids of an N-glycosylation site, but the N-glycosylation site has been modified such that the glycoprotein does not contain a glycan longer than two monosaccharide residues within 10 amino acids of the exposed tyrosine residue; (b) converting the phenol moiety of the exposed tyrosine residue into an ortho-quinone moiety by contacting the glycoprotein with an oxidative enzyme capable of oxidizing tyrosine; and (c) reacting the ortho-quinone moiety with an alkene or alkyne compound via a [4+2] cycloaddition, wherein the compound comprises a (hetero)cycloalkene or (hetero)cycloalkyne moiety and (i) a chemical handle to further modify the compound with a payload, or (ii) a payload. The resulting glycoprotein-conjugates and pharmaceutical compositions and methods of treatment comprising same are also disclosed.

### CROSS-REFERENCE TO RELATED APPLICATIONS

This application is a continuation of International Patent Application No. PCT/NL2021/050714 filed Nov. 22, 2021, which application claims priority to Netherlands Patent Application No. 2026947 filed Nov. 20, 2020, the contents of which are all incorporated herein by reference in their entireties.

### SEQUENCE LISTING

The present application is filed along with a Sequence Listing in electronic format. The Sequence Listing is provided as a file entitled 2023-09-05_069818-1090_Sequence_Listing.xml, created on Aug. 31, 2023, which is 14,845 bytes in size. The information in the electronic format of the Sequence Listing is incorporated herein by reference in its entirety.

### FIELD OF THE INVENTION

The present invention relates to the field of antibody-drug conjugates, in particular to antibody-drug conjugates prepared by tyrosinase-mediated bioconjugation, which are suitable for the treatment of cancer.

### BACKGROUND OF THE INVENTION

Antibody-drug conjugates (ADC), considered as magic bullets in therapy, are comprised of an antibody to which is attached a pharmaceutical agent. The antibodies (also known as ligands) are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated payload to the tumour, via binding, internalization, intracellular processing and finally release of active catabolite. The payload may be a small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases. Finally, attachment of an oligonucleotide to an antibody selectively taken up by muscle cells is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

In the field of ADCs, a chemical linker is typically employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment). The cleavable linker may also contain a self-immolative unit, for example based on a para-aminobenzyl alcohol group and derivatives thereof. A linker may also contain an additional, non-functional element, often referred to as spacer or stretcher unit, to connect the linker with a reactive group for reaction with the antibody.

Currently, cytotoxic payloads include for example microtubule-disrupting agents [e.g. auristatins such as monomethyl auristatin E (MMAE) and monomethyl auristatin F (MMAF), maytansinoids, such as DM1 and DM4, tubulysins], DNA-damaging agents [e.g., calicheamicin, pyrrolobenzodiazepine (PBD) dimers, indolinobenzodiapine dimers, duocarmycins, anthracyclines], topoisomerase inhibitors [e.g. DXd, SN-38] or RNA polymerase II inhibitors [e.g. amanitin]. ADCs that have reached market approval include for example payloads MMAE, MMAF, DM1, calicheamicin, SN-38 and DXd, while various pivotal trials are running for ADCs based on duocarmycin, DM4 and PBD dimer. A larger variety of payloads is still under clinical evaluation or has been in clinical trials in the past, e.g. eribulin, indolinobenzodiazepine dimer, PNU-159,682, hemi-asterlin, doxorubicin, vinca alkaloids and others. Finally, various ADCs in late-stage preclinical stage are conjugated to novel payloads for example amanitin, KSP inhibitors, MMAD, and others.

With the exception of sacituzumab govetican (Trodelvy®), all of the clinical and marketed ADCs contain cytotoxic drugs that are not suitable as stand-alone drug. Trodelvy® is the exception because it features SN-38 as cytotoxic payload, which is also the active catabolite of irinotecan (an SN-38 prodrug). Several other payloads now used in clinical ADCs have been initially evaluated for chemotherapy as free drug, for example calicheamicin, PBD dimers and eribulin. but have failed because the extremely high potency of the cytotoxin (picomolar-low nanomolar IC₅₀ values) versus the typically low micromolar potency of standard chemotherapy drugs, such as paclitaxel and doxorubicin.

Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948, incorporated by reference, and for example studied by Li et al, Cancer Res. 2016, 76, 2710-2719, incorporated by reference. Generally spoken, cytotoxic payloads that are neutral will show bystander killing whereas ionic (charged) payloads do not, as a consequence of the fact that ionic species do not readily pass a cellular membrane by passive diffusion. Payloads with established bystander effect are for example MMAE and DXd. Examples of payloads that do not show bystander killing are MMAF or the active catabolite of Kadcyla (lysine-MCC-DM1).

ADCs are prepared by chemical attachment of a reactive linker-drug to a protein, a process known as bioconjugation. Many technologies are known for bioconjugation, as summarized in G. T. Hermanson, “Bioconjugate Techniques”, Elsevier, 3^{rd} Ed. 2013, incorporated by reference. Two main technologies can be recognized for random conjugation to antibodies, either based on acylation of lysine side chain or based on alkylation of cysteine side chain. Acylation of the ε-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in Adcetris©. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, DOI: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/ (accessed Jan. 7, 2020). An alternative approach to antibody conjugation via cysteine involves the addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ˜4 (DAR4). Another useful technology for conjugation to a cysteine side chain is by means of disulfide bond, a bioactivatable connection that has been utilized for reversibly connecting protein toxins, chemotherapeutic drugs, and probes to carrier molecules (see for example Pillow et al., Chem. Sci. 2017, 8, 366-370, incorporated by reference).

A frequent method for attachment of linker-drugs to azido-modified proteins is strain-promoted alkyne-azide cycloaddition (SPAAC). In a SPAAC reaction, the linker-drug is functionalized with a cyclic alkyne and the cycloaddition with azido-modified antibody is driven by relief of ring-strain. Conversely, the linker-drug is functionalized with azide and the antibody with cyclic alkyne. Various strained alkynes suitable for metal-free click chemistry are indicated in FIG. 1. Besides cyclooctyne, certain cycloheptynes are also suitable for metal-free click chemistry, as reported by Weterings et al., Chem. Sci. 2020, doi: 10.1039/d0sc03477k, incorporated by reference. Smaller strained alkynes may also be employed, however in most cases require in situ generation of the strained alkyne due to inherent instability.

Reaction of strained alkynes with tetrazine is also a metal-free click reaction. Moreover, tetrazines also react with strained alkenes (tetrazine ligation). Both strained alkynes and strained alkenes react with tetrazines via inverse electron-demand Diels-Alder (IEDDA) reactions, exhibiting remarkably fast kinetics. For example, reaction of trans-cyclooctene (TCO) with tetrazine is unrivalled in its reaction speed and such rapid reaction has enabled applications in rodent models and other large organisms, settings where only minimal reaction times and reagent concentrations are tolerated. Triazine and other heteroaromatic moieties can also undergo reaction with strained alkynes or alkenes. Notably, strained alkenes typically do not undergo reaction with azides. Various strained alkenes suitable for metal-free click chemistry are indicated in FIG. 2.

Besides azides, strained alkynes can also undergo reaction with a range of other functional groups, such as nitrile oxide, nitrone, ortho-quinone, dioxothiophene and sydnone. A list of couples of functional groups F and Q (=strained alkyne or strained alkene) for metal-free click chemistry is provided in FIG. 3. A comprehensive overview of metal-free click chemistries for bioconjugation, extending also beyond proteins (e.g. glycans, nucleic acids), is provided by Nguyen and Prescher, Nature rev. 2020, doi: 10.1038/s41570-020-0205-0, incorporated by reference.

Based on the above, a general method for the preparation of a protein conjugate, exemplified for a monoclonal antibody in FIG. 4, entails the reaction of a protein containing x number of reactive moieties F with a linker-drug construct containing a single molecule Q.

Introduction of an azide or a tetrazine moiety onto a protein can be achieved by genetic encoding, by enzymatic installation or by chemical acylation. One method is based on genetic encoding of a non-natural amino acid, e.g. p-acetophenylalanine suitable for oxime ligation, or p-azidomethylphenylalanine or p-azidophenylalanine suitable for click chemistry conjugation, as for example demonstrated by Axup et al. Proc. Nat. Acad. Sci. 2012, 109, 16101-16106, incorporated by reference. Similarly, Zimmerman et al., Bioconj. Chem. 2014, 25, 351-361, incorporated by reference have employed a cell-free protein synthesis method to introduce azidomethylphenylalanine (AzPhe) into monoclonal antibodies for conversion into ADC by means of metal-free click chemistry. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of (copper-catalysed) click chemistry. Finally, genetic encoding of aliphatic azides in recombinant proteins using a pyrrolysyl-tRNA synthetase/tRNA_{CUA} pair was shown by Nguyen et al., J. Am. Chem. Soc. 2009, 131, 8720-8721, incorporated by reference and labelling was secured by click chemistry.

Another method is based on enzymatic installation of a non-natural functionality. For example, Dennler at al., Bioconj. Chem. 2014, 25, 569-578 and Lhospice et al., Mol. Pharmaceut. 2015, 12, 1863-1871, both incorporated by reference, employ the bacterial enzyme transglutaminase (BTG or TGase) for installation of an azide moiety onto an antibody. To this end, the key glutamine residue for TGase-mediated installation is first liberated by PNGase F-mediated removal of the native N-glycan, as first demonstrated by Jeger et al., Angew. Chem. Int. Ed. 2010, 49, 9995-9997, incorporated by reference. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

It has been shown by van Geel et al., Bioconj. Chem. 2015, 26, 2233-2242 and Verkade et al., Antibodies 2018, 7, 12, all incorporated by reference, that enzymatic remodelling of the native antibody glycan at N297 also enables introduction of an azide into the antibody by means of an azido sugar, suitable for attachment of cytotoxic payload using click chemistry. Chemical approaches have also been developed for site-specific modification of antibodies without prior genetic modification, as for example highlighted by Yamada and Ito, ChemBioChem. 2019, 20, 2729-2737.

Of the functional moieties F in FIG. 3, azide and nitrone can be installed onto a natural protein also by chemical modification. The resulting azide- or nitrone-containing protein can then undergo metal-free click conjugation with a suitable probe Q, providing the resulting protein conjugate in a straightforward two-stage process. For example, treatment of a natural protein with a diazo transfer reagent leads to chemical conversion of free amino groups to azide groups, as was for example reported by Schoffelen et al., Chem. Sci. 2011, 2, 701-705, incorporated by reference. Moreover, careful titration of the pH in some cases leads to selective conversion of the amine with the lowest pKa (typically the amine at the N-terminus of the model proteins). The resulting azides were modified based on strain-promoted cycloaddition of functionalized cycloalkynes. Also, it was shown by Ning et al., Angew. Chem. Int. Ed. 2010, 49, 3065-3068, incorporated by reference, that an N-terminal nitrone can be generated onto a natural polypeptide by periodate-mediated oxidation of an N-terminal serine or threonine, followed by treatment with excess of N-alkyl hydroxylamine. The resulting nitrone was shown to undergo rapid in situ cycloaddition with a strained alkyne.

Of the functional moieties F in Table 3, an ortho-quinone can be generated directly from a natural protein by oxidation of tyrosine side chain, as reviewed by Bruins et al., Chem. Eur. J. 2017, 24, 4749-4756, incorporated by reference. A main advantage of the generation of an ortho-quinone versus azide or nitrone is the fact that the ortho-quinone is able to undergo in situ follow-up chemistry to generate the protein conjugate in a one-stage process without isolation of the quinone intermediate. For example, it was reported by Wilchek and Miron, Bioconj. Chem. 2015, 26, 502, incorporated by reference, that direct chemical conversion of phenol group in tyrosine to ortho-quinone can be achieved by treatment with potassium nitrosodisulfonate (PTN, also known as Fremy's salt) and used it for protein polymerization. Similarly, George et al., ChemistrySelect 2017, 2, 7117-7112, incorporated by reference, showed that strain-promoted oxidation-controlled cyclooctyne-1,2-quinone cycloaddition (SPOCQ) can be employed for protein modification by generation of ortho-quinone with Fremy's salt followed by in situ reaction with bicyclononyne (BCN), as highlighted in FIG. 5. However, the use of strong oxidants like Fremy's salt can result in collateral oxidation of cysteine and methionine side chains, and oxidants needs to be removed from the protein solution following the reaction. Besides, multiple tyrosine moieties may undergo oxidation to ortho-quinone, thereby leading to a heterogeneous mixture of protein conjugates.

One elegant solution to circumvent chemical oxidants is the use of an enzyme to generate an ortho-quinone. Tyrosinase- and phenol oxidase-mediated generation of ortho-quinones has been known for decades to mediate cross-linking between proteins in meat, whey and flour via non-selective tyrosine-tyrosine, tyrosine-cysteine, and tyrosine-lysine linkages. By performing the enzyme-mediated generation of the ortho-quinone in the presence of a suitable external nucleophile, the oxidized protein will readily undergo chemical conjugation, as for example demonstrated by Struck et al., J. Am. Chem. Soc. 2016, 138, 3038-3045. A disadvantage of enzymatic oxidation of proteins is that the majority or all of the tyrosine moieties are typically buried in the hydrophobic interior of the protein and therefore not be accessible for a bulky enzyme like tyrosinase. On the other hand, the absence of a native tyrosine for oxidation has paved the way for selective peripheral protein oxidation by the introduction of an N- or C-terminal fusion tag with an exposed tyrosine. For example, it was shown by Bruins et al., Bioconj. Chem. 2017, 28, 1189-1193, incorporated by reference, that laminarase A, a hyperstable endo-β-1,3-glucanase, could be selectively fluorophore-modified upon SPOCQ by fusion of C-terminal G₄Y-tag onto the glucanase, while the same C-terminal G₄Y-tag fused to trastuzumab light chain enabled the generation of a site-specific antibody-drug conjugate upon reaction with BCN-linker-MMAE. Bruins et al. have also demonstrated, Chem. Commun. 2018, 54, 7338-7341, incorporated by reference, that an antibody-drug conjugate could be generated by reaction of the C-terminal ortho-quinone with a linker-auristatin construct based on conformationally strained trans-cyclooctene (sTCO). A similar approach was most recently reported by Marmelstein et al., J. Am. Chem. Soc. 2020, 142, 5078-5086, incorporated by reference, showing that a C-terminal GGY tag on trastuzumab single chain enables selective tyrosinase-mediated coupling of various tags.

The introduction of functionality F in all cases described above requires either genetic modification of the protein (genetic encoding of non-natural amino acid, introduction of specific fusion tag) or a two-stage approach where the functionality F is first introduced chemically or enzymatically. However, there currently exists no generic method for the one-step modification of native proteins based on modification of natural amino acid side-chains by means of metal-free click chemistry.

### SUMMARY OF THE INVENTION

The present inventors have surprisingly found that natural N-glycoprotein are not sensitive to oxidative enzymes like tyrosinase or (poly)phenol oxidase, however if the native N-glycan is modified, e.g. (a) removed, e.g. by PNGase F hydrolysis, or (b) trimmed, e.g. by endoglycosidase, or (c) mutated to another amino acid, a nearby tyrosine residue of the glycoprotein becomes exposed, and susceptible to oxidative enzymes, leading to the formation of ortho-quinone (FIG. 6). By performing the enzymatic oxidation in the presence of a (functionalized) strained alkyne or alkene (exemplary structures in FIG. 7), the resulting ortho-quinone can undergo in situ [4+2]cycloaddition with a strained alkyne or strained alkene, thereby forming an glycoprotein conjugate in a one-pot process.

The invention first and foremost concerns conjugates having structure (1a) or (1b):

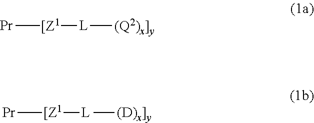

wherein:
Pr is an N-glycoprotein;
Z¹ comprises structure (Za) or (Zb):

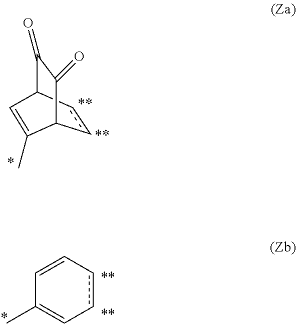

wherein the carbon labelled with * is directly connected to the peptide chain of the antibody at an amino acid located within 10 amino acids of an N-glycosylation site, which has been modified such that the glycoprotein does not contain a glycan longer than two monosaccharide residues within 10 amino acids of the amino acid residue, and both of the carbon atoms labelled with ** are connected to L, and the bond depicted as [symbol] is a single bond or a double bond;
L is a linker;
x is an integer in the range of 1-4;
y is an integer in the range of 1-4;
Q² is a chemical handle that is reactive towards an appropriately functionalized payload;
D is a payload.

The invention further concerns a process for the synthesis of the conjugate according to the invention, the medical use of the conjugate according to the invention and a pharmaceutical composition comprising the conjugate according to the invention.

### DESCRIPTION OF THE FIGURES

FIG. 1 shows cyclic alkynes suitable for metal-free click chemistry, and preferred embodiments for reactive moiety Q¹ and Q². The list is not comprehensive, for example alkynes can be further activated by fluorination, by substitution of the aromatic rings or by introduction of heteroatoms in the aromatic ring.

FIG. 2 shows cyclic alkenes suitable for metal-free click chemistry, and preferred embodiments for reactive moiety Q¹ and Q². The list is not comprehensive, for example alkenes can be further activated by fluorination, by introduction of (hetero)aromatic rings, which may be further substituted.

FIG. 3 shows a representative (but not comprehensive) set of functional groups (F) that can be introduced into a glycoprotein by engineering, by chemical modification, or by enzymatic means, which upon metal-free click reaction with a complementary reactive group Q lead to connecting group Z. Functional group F may be introduced into a glycoprotein at any position of choice by engineering, chemical or enzymatic modification. Various functional groups are known to react exclusively or with high preference with strained alkynes (azide, sydnone). Other functional groups F (nitrile oxide, nitrone, quinone, dioxothiophene, tetrazine, triazine) are reactive with both strained alkynes and strained alkenes. The bicyclic cycloadduct formed by reaction of ortho-quinone or dioxothiophene with strained alkyne may eliminate CO or SO₂, respectively, to form an aromatic ring. Similar elimination may also occur after cycloaddition with strained alkene, however will also require subsequent oxidation of the intermediate dihydrobenzene ring. The pyridine or pyridazine connecting group is the product of the rearrangement of the tetrazabicyclo[2.2.2]octane connecting group, formed upon reaction of triazine or tetrazine with alkyne (but not alkene), respectively, with loss of N₂. Similar functional groups (F) are normally present or can be introduced into a payload, for conjugation by metal-free click reaction with a complementary reactive group Q leading to connecting group Z.

FIG. 4 shows the general scheme for preparation of antibody-drug conjugates by reaction of a monoclonal antibody (in most cases a symmetrical dimer) containing an x number of functionalities F. By incubation of antibody-(F)ₓ with excess of a linker-drug construct (Q-spacer-linker-payload) a conjugate is obtained by reaction of F with Q, forming connecting group Z.

FIG. 5 depicts the general concept of oxidation-mediated generation of an ortho-quinone on a protein, followed by in situ [4+2] cycloaddition with a suitable strained alkyne (for example BCN).

FIG. 6 depicts the lack of reactivity of native antibodies that are N-glycosylated in the C_{H}2 domain (e.g. N297) for enzymatic oxidation (arrow to the left), however upon removal of the entire glycan (with PNGase F or other hydrolases N297 glycan is hydrolysed, leaving asparate-297) or trimming of the glycan (with endoglycosidase) the antibody becomes susceptible to tyrosinase-mediated oxidation of the neighbouring tyrosine.

FIG. 7 depicts representative structures linker-payloads suitable for cycloaddition with ortho-quinones. For example, the linker-payload may be functionalized with a strained alkyne like BCN, may contain one or more units of a carbamoyl sulfamide, may be branched and may contain a cleavable linker based on valine-citrulline or valine-alanine (all top structure). Alternative, the linker-payload may be functionalized with a strained alkene like sTCO and may contain non-cleavable linker (bottom structure).

FIG. 8 shows the amino acid sequences of the C_{H}2 constant domain of human IgG1, IgG2, IgG3 and IgG4 and mouse IgG1, IgG2ab, IgG2aa, IgG2b, IgG3. Native glycosylation site (N) is underlined, tyrosine moieties (Y) that can undergo oxidation after the glycan is removed or truncated in bold italics.

FIG. 9 depicts the structures of various functionalized click reagents for conjugation to ortho-quinone functionality (e.g. dyes, ODNs, proteins).

FIG. 10 depicts the structures of BCN-linker-payloads with MMAE (6a and 6b) or PBD (7).

FIG. 11 depicts the structure of bifunctional reagent 8, functionalized with a strained alkyne (BCN) as well as a strained alkene (TCO). Also depicted are the structures of various methyltetrazine-modified reporter molecules, i.e. TAMRA (9a), IL-2 (9b), UCHT1 (9c) and ODN1826 (9d).

FIG. 12 shows the reducing SDS-PAGE for trastuzumab (lane I), PNGase F deglycosylated trastuzumab (lane II) and deglycosylated trastuzumab after treatment with mushroom tyrosinase in the presence BCN-lissamine 1 (lane III). Picture on the left=Coomassie staining, picture on the right=fluorescence image. A fluorescent band is apparent only for trastuzumab upon deglycosylation and treatment with tyrosinase in the presence of 1.

FIG. 13 depicts the MS data for the Fc-fragment of IdeS-treated trastuzumab (top), PNGase F deglycosylated trastuzumab (middle) and deglycosylated trastuzumab after treatment with mushroom tyrosinase in the presence BCN-lissamine 1 (bottom). Picture on the left shows full range (0-100,000 Da), picture on the right is zoom (23,000-27,000).

FIG. 14 depicts the MS data for the Fc-fragment of IdeS-treated cetuximab (top), PNGase F deglycosylated cetuximab (middle) and deglycosylated cetuximab after treatment with mushroom tyrosinase in the presence BCN-lissamine 1 (bottom). Picture on the left shows full range (0-100,000 Da), picture on the right is zoom (23,000-27,000).

FIG. 15 depicts the relationship between stoichiometry of BCN-lissamine (1) versus deglycosylation trastuzumab in the presence of mushroom tyrosinase. Clean conversion into a new product (retention time 8.7 min) is achieved with minimum of 2.5 equiv. of 1 (t=6.4 min=LC; t=8.3 min=HCO; t=8.7 min=HCl).

FIG. 16 shows the reducing SDS-PAGE for labelling to deglycosylated trastuzumab and cetuximab upon treatment with tyrosinase in the presence of TCO-AF568 (3).

FIG. 17 shows the HPLC-traces for deglycosylated trastuzumab (top), after reaction with BCN-lissamine (1) in the presence of tyrosinase (middle) and after reaction with TCO-AF568 (3) in the presence of tyrosinase (bottom) (t=6.6 min=LC; t=7.9 min=HCO; t=8.1 min=HCl (with 3); t=8.6 min=HCl (with 1)).

FIG. 18 shows the lack of fluorescence labelling for mouse IgG1 and human IgG2 before and after deglycosylation and tyrosinase in the presence of 3 treatment due to the lack of native tyrosine in the vicinity of the native glycosylation site.

FIG. 19 shows the HPLC-trace for a competition experiment (bottom trace D) for labelling of trastuzumab-LC-G₄Y (trace A) in the presence of both BCN-lissamine (1) and TCO-AF568 (3), showing the clear formation of primarily the adduct of LC-G₄Y and 1 (t=7.1 min) and minute amounts of LC-G₄Y adduct with 3 (t=6.4 min). Separate experiments depicted for 1 (trace B) and 3 (trace C). In all traces shown, t=6.2 corresponds to the unmodified LC-G₄Y (LC0) and t=7.4 min corresponds to the unmodified HCO.

FIG. 20 depicts the strategy for conversion of an antibody into a TCO-labelled antibody by (a) deglycosylation, and (b) treatment with bifunctional BCN-TCO reagent 8 in the presence of tyrosinase.

FIG. 21 shows the results of treatment of trastuzumab-TCO (depicted in FIG. 8, lane A) with reagents 9a-9d (lanes B-E) by reducing SDS-PAGE (Coomassie staining and fluorescence imaging). Formation of new bands with higher molecular weight than HC is visible by Coomassie staining for reagents 9a-9c, while a fluorescent band for HC becomes visible of 9d.

FIG. 22 shows MS data and RP-HPLC data for PNGase F-deglycosylated trastuzumab.

FIG. 23 shows MS data and RP-HPLC data for PNGase F-deglycosylated B12.

FIG. 24 shows SEC data, MS data and RP-HPLC data for PNGase F-deglycosylated trastuzumab, after treatment with BCN-MMAE (6a).

FIG. 25 shows SEC data, MS data and RP-HPLC data for PNGase F-deglycosylated trastuzumab, after treatment with BCN-MMAE₂ (6b).

FIG. 26 shows SEC data, MS data and RP-HPLC data for PNGase F-deglycosylated trastuzumab, after treatment with BCN-PBD (7). Note: *BCN-HS-PEG₂-va-PABC-PBD is not stable under the acidic conditions used in sample work-up and analysis. Therefore, some peak broadening is observed, so conversion was determined by the amount of starting material left.

FIG. 27 shows SEC data, MS data and RP-HPLC data for PNGase F-deglycosylated B12, after treatment with BCN-MMAE₂ (6b).

FIG. 28 shows the in vitro efficacy on HER2-positive cell line SK-BR-3 of various antibody conjugates, prepared from trastuzumab or B12 (negative control). As a positive control is included GC-ADC: BCN-MMAE 6a conjugated to trastuzumab after enzymatic remodelling with 6-azidoGalNAc (according to WO2016170186, incorporated by reference).

FIG. 29 shows the RP-HPLC analysis of the antibody conjugate obtained by endoglycosidase trimming of trastuzumab (mostly fucosylated) followed by incubation with tyrosinase and linker-payload 6a.

FIG. 30 shows mass spectrometry data of the antibody conjugate obtained by trimming of high-mannose trastuzumab (non-fucosylated) followed by incubation with tyrosinase and linker-payload 6a.

FIG. 31 shows the RP-HPLC analysis of the antibody conjugate obtained by trimming of high-mannose trastuzumab (non-fucosylated) followed by incubation with tyrosinase and linker-payload 6a.

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as R and S stereoisomers. Unless stated otherwise, the description of any compound in the description and in the claims is meant to include both the individual R and the individual S stereoisomers of a compound, as well as mixtures thereof. When the structure of a compound is depicted as a specific S or R stereoisomer, it is to be understood that the invention of the present application is not limited to that specific S or R stereoisomer.

The compounds disclosed in this description and in the claims may further exist as R and S stereoisomers. Unless stated otherwise, the description of any compound in the description and in the claims is meant to include both the individual R and the individual S stereoisomers of a compound, as well as mixtures thereof. When the structure of a compound is depicted as a specific S or R stereoisomer, it is to be understood that the invention of the present application is not limited to that specific S or R stereoisomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically acceptable” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

A “conjugate” is herein defined as a compound wherein an antibody is covalently connected to a payload via a linker. A conjugate comprises one or more antibodies and/or one or more payloads.

The term “payload” refers to the moiety that is covalently attached to a targeting moiety such as an antibody, but also to the molecule that is released from the conjugate upon uptake of the protein conjugate and/or cleavage of the linker. Payload thus refers to the monovalent moiety having one open end which is covalently attached to the targeting moiety via a linker and also to the molecule that is released therefrom. In the context of the present invention, the payload is exatecan.

The terms “tyrosinase” and “(poly)phenol oxidase” refer to an enzyme that is capable of catalysing the ortho-hydroxylation of a monophenol moiety to an ortho-dihydroxybenzene (catechol) moiety, followed by further oxidation of the ortho-dihydroxybenzene moiety to produce an ortho-quinone (1,2-quinone) moiety.

The term “deglycosylation” refers to the treatment of an N-glycoprotein with an amidase to remove the entire glycan, i.e. by enzymatic hydrolysis of the amide bond between the amino acid, usually asparagine, of the protein and the first monosaccharide, usually GlcNAc, at the reducing end of the glycan.

The term “deglycosylated protein” refers to an N-glycoprotein that has been treated with an amidase to remove the entire glycan, i.e. by enzymatic hydrolysis of the amide bond between the amino acid, usually asparagine, of the protein and the first monosaccharide, usually GlcNAc, at the reducing end of the glycan.

The term “trimming” refers to the treatment of an N-glycoprotein with an endoglycosidase to hydrolyse the glycosidic bond between the first monosaccharide, usually GlcNAc, at the reducing end of the glycan, which is attached to an amino acid, usually asparagine, and the second monosaccharide, usually GlcNAc.

The term “trimmed protein” refers to an N-glycoprotein that has been treated with an endoglycosidase to hydrolyse the glycosidic bond between the first monosaccharide, usually GlcNAc, at the reducing end of the glycan, which is attached to an amino acid, usually asparagine, and the second monosaccharide, usually GlcNAc.

### The Invention

The inventors have found that N-glycoproteins that are normally not reactive towards enzymatic oxidation of a tyrosine residue, by enzymes such as tyrosinase or (poly)phenol oxidase, can be made reactive by shortening or removing the glycan. This finding provides a new opportunity for preparing glycoprotein conjugates, as these tyrosine residues can now readily be converted into ortho-quinone moieties, which are in turn chemical handles that can be reacted with (hetero)cycloalkene or (hetero)cycloalkyne moieties. Thus, when the ortho-quinone moiety is reacted with a compound comprising a (hetero)cycloalkene or (hetero)cycloalkyne moiety, a covalent attachment is formed between the N-glycoprotein and that compound, and when that compound further comprises (i) a chemical handle to further modify the compound with a payload, or (ii) a payload itself, a conjugate of the N-glycoprotein and a payload is readily formed.

In a first aspect, the invention concerns a process for the preparation of N-glycoprotein-conjugates. The process according to the invention comprises:
(a) providing an N-glycoprotein having an exposed tyrosine residue, wherein the exposed tyrosine residue is located within 10 amino acids of an N-glycosylation site, but that N-glycosylation site has been modified such that the glycoprotein does not contain a glycan longer than two monosaccharide residues within 10 amino acids of the exposed tyrosine residue;
(b) converting the phenol moiety of the exposed tyrosine residue into an ortho-quinone moiety by contacting the antibody with an oxidative enzyme capable of oxidizing tyrosine;
(c) reacting the ortho-quinone moiety with an alkene or alkyne compound via a [4+2] cycloaddition, wherein the compound comprises a (hetero)cycloalkene or (hetero)cycloalkyne moiety and (i) a chemical handle to further modify the compound with a payload, or (ii) a payload.

The invention further concerns conjugates obtainable by the process according to the invention. The conjugate according to the invention may also be defined as having structure (1a) or (1b):

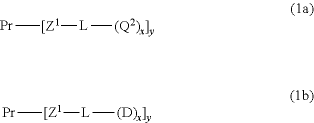

wherein:
Pr is an N-glycoprotein;
Z¹ comprises structure (Za) or (Zb):

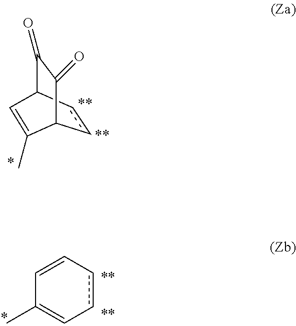

wherein the carbon labelled with * is directly connected to the peptide chain of the antibody at an amino acid located within 10 amino acids of an N-glycosylation site, which has been modified such that the glycoprotein does not contain a glycan longer than two monosaccharide residues within 10 amino acids of the amino acid residue, and both of the carbon atoms labelled with ** are connected to L, and the bond depicted as [symbol] is a single bond or a double bond;
L is a linker;
x is an integer in the range of 1-4;
y is an integer in the range of 1-4;
Q² is a chemical handle that is reactive towards an appropriately functionalized payload;
D is a payload.

The invention further concerns a process for the synthesis of the conjugate according to formula (1b) from a conjugate according to formula (1a), the medical use of the conjugate according to formula (1b) and a pharmaceutical composition comprising the conjugate according to formula (1b).

### The N-glycoprotein

The N-glycoprotein that is provided in step (a) contains an exposed tyrosine residue. A tyrosine residue is considered to be exposed in the context of the present invention as it would normally be located within 10 amino acids of an N-glycosylation site, but that N-glycosylation site has been modified such that the glycoprotein does not contain a glycan longer than two monosaccharide residues within 10 amino acids of the exposed tyrosine residue. In other words, this N-glycosylation site does not contain a glycan longer than two monosaccharide residues. Herein, “within X amino acids” refers to maximally X-1 amino acids located in between the exposed tyrosine residue and the (modified) N-glycosylation site, such that exposed tyrosine residue is at most the X^{th} amino acids counting from the glycosylated amino acid. Thus, the exposed tyrosine residue is located within 10 amino acids of a native N-glycosylation site. Such a native N-glycosylation site is typically at a asparagine residue. Preferably, the exposed tyrosine residue is located within 8 amino acids, more preferably within 5 amino acids or even within 3 amino acids, of such an N-glycosylation site. The exposed tyrosine residue being located within 10 amino acids of the native N-glycosylation site could also refer to a tyrosine residue which is introduced, e.g. by point mutation, at the position of the N-glycosylated amino acid, usually an asparagine residue. By introduction of a tyrosine residue in lieu of the asparagine residue, the N-glycan will be absent, i.e. a glycan having no monosaccharide residues, and the introduced tyrosine residue fulfils the location requirements of being within 10 amino acids of the native N-glycosylation site.

N-glycan structures at the glycosylation site may come in various isoforms (e.g. G0, G1, G2), which have at least 5 monosaccharide residues, but typically much more such as at least 7. These large glycans block nearby tyrosine residues from being reactive towards oxidative enzymes, and these tyrosine residues are made available (“exposed”) for such enzymes. The phenolic side chains of tyrosine residues are usually folded towards the interior of proteins, such that they are not reactive towards oxidative enzymes. However, the phenolic side chains of tyrosine residues nearby an N-glycosylation site typically point towards to outside of the protein, such that they may be reactive towards oxidative enzymes if the glycan would not be in the way. This is particularly true for antibodies, which normally have one or two tyrosine residues located nearby an N-glycosylation site, which are exposed for reaction with oxidative enzymes in step (b) of the process according to the invention.

The glycoprotein may not have a glycan longer than two monosaccharide residues within 10 amino acids of the exposed tyrosine residue. Preferably, such a glycan is not present within 15 amino acids or even within 20 amino acids. Most preferably, the glycoprotein does not comprise a glycan longer than two monosaccharide residues at all. Typically, this refers to the glycan at the native N-glycosylation site. The inventors found that glycans of at most two monosaccharide residues may be present within this range around the exposed tyrosine residue, and the reaction of step (b) will still take place, whereas such tyrosine residues would be blocked (i.e. not exposed) if the glycan would be longer. Both partial (leaving up to two monosaccharide residues) and complete removal of the glycan is found to expose the otherwise blocked tyrosine residue and make it available for reaction as in step (b). In a preferred embodiment, the glycan is completely absent or has the structure -GlcNAc(Fuc)_{b}, wherein b is 0 or 1. Herein, the GlcNAc moiety is directly attached to a nitrogen atom of an amino acid in the peptide chain of the glycoprotein, mostly to the amide nitrogen of an asparagine residue. Such a GlcNAc moiety is referred to as a core GlcNAc moiety. The core GlcNAc moiety may be further substituted at its 6-OH by α-Fuc, in which case b=1. Such optional fucosylation of the core GlcNAc moiety is a common feature of antibodies, and in the context of the present invention the presence of the fucosyl moiety is irrelevant.

Since the N-glycan(s) of the N-glycoprotein may be completely removed in step (a), the N-glycoprotein having an exposed tyrosine residue may not contain an N-glycan at all. Since the tyrosine residue(s) was/were originally blocked by the glycan(s), the protein that remains after removal of the glycan(s) is still referred to as an N-glycoprotein in the context of the present invention.

The original or native N-glycoprotein that is used in the process according to the invention may have more than one tyrosine residue. It is preferred that the N-glycoprotein only contains blocked tyrosine residues before being exposed. It is thus preferred that the N-glycoprotein, before the tyrosine residue(s) is/are exposed, is unreactive towards an oxidative enzyme capable of oxidizing tyrosine, such as tyrosinase or (poly)phenol oxidase. Alternatively, the N-glycoprotein may also contain one or more tyrosine residues that are reactive towards an oxidative enzyme capable of oxidizing tyrosine even without modification of the N-glycan. The process according to the invention is still beneficial for such glycoproteins, as one or more additional tyrosine residues become available as conjugation site, thus enabling the preparation of glycoprotein conjugates with higher payload loading. The N-glycoprotein preferably comprises 1-4 exposed tyrosine residues, more preferably the glycoprotein comprises 1, 2 or 4 exposed tyrosine residues, most preferably the glycoprotein comprises 2 or 4 exposed tyrosine residues. This number is also denoted as y in the definition of the conjugate. The tyrosine residue(s) that is/are exposed may be introduced by genetic modification of the N-glycoprotein, or preferably is/are located at the native position.

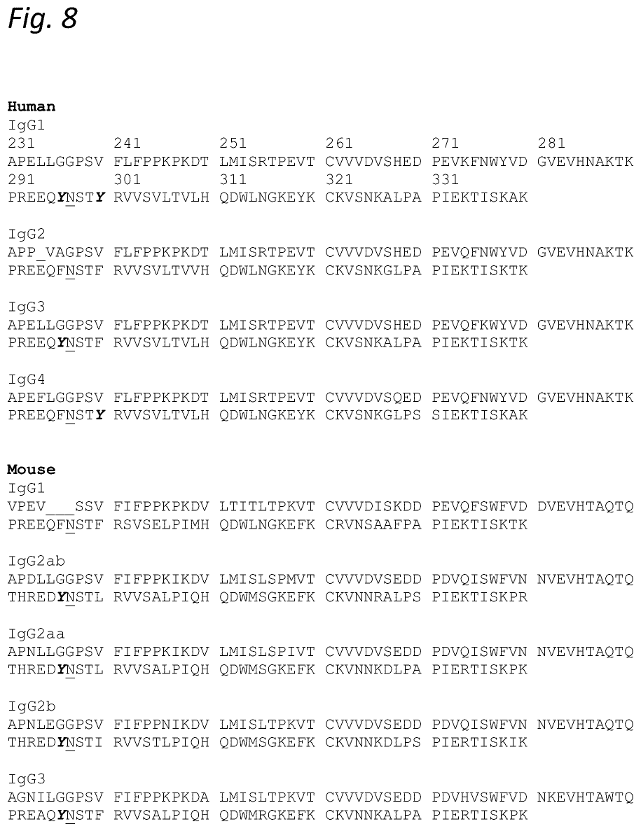

In a preferred embodiment, the N-glycoprotein is an antibody, preferably a recombinant antibody, generated in mammalian host systems. Antibodies normally have a conserved N-glycosylation site at (or around) asparagine-297 (N297), as part of the consensus sequence of N-glycosylation NST, see also FIG. 8. Glycan structures of various isoforms (e.g. G0, G1, G2) may be present at this glycosylation site, which may have 12 to 18 monosaccharide residues. These large glycans block nearby tyrosine residue from being reactive towards oxidative enzymes. Thus, in case the N-glycoprotein is an antibody, it is preferred that the N-glycosylation site is the glycosylation site at or around position 297 of the amino acid sequence of the antibody, such as at a position in the range of 294-300, preferably in the range 295-298, most preferably at position 297. The exposed tyrosine residue is thus located within 10 amino acids of that N-glycosylation site, preferably within 8 amino acids, more preferably within 5 amino acids, most preferably within 3 amino acids. It is thus preferred that the exposed tyrosine residue is located at an amino acid position in the range of 284-310, preferably in the range of 287-307, preferably in the range of 289-305, more preferably in the range of 292-302, most preferably in the range of 294-300 of the amino acid sequence of the antibody. More specifically, in case the N-glycoprotein is an antibody, it is preferred that the N-glycosylation site is the glycosylation site at or around N297 and the exposed tyrosine residue is located within 10 amino acids of that N-glycosylation site, preferably within 8 amino acids, more preferably within 5 amino acids, most preferably within 3 amino acids. Even more specifically, in case the N-glycoprotein is an antibody, it is preferred that the N-glycosylation site is the glycosylation site at N297 and the exposed tyrosine residue is located within 10 amino acids of that N-glycosylation site, i.e. at a position in the range of 287-307, preferably within 8 amino acids, i.e. at a position in the range of 289-305, more preferably within 5 amino acids, i.e. at a position in the range of 292-302, most preferably within 3 amino acids, i.e. at a position in the range of 294-300 of the amino acid sequence of the antibody. Preferably, the tyrosine residue at position Y296 and/or Y300 is exposed. Preferred amino acid sequences are depicted in FIG. 8.

The exposed tyrosine residue may be located at a native position, i.e. at the position of a tyrosine residue in the amino acid sequence of the native N-glycoprotein, or at a non-native position, wherein a tyrosine residue is introduced at a position within 10 amino acids of an N-glycosylation site. Such point mutations wherein a specific amino acid residue is introduced at a specific site in the amino acid sequence of a protein is well-known in the art. Preferably, native tyrosine residues are used as exposed tyrosine residues in the context of the present invention.

The N-glycoprotein having the exposed tyrosine residue may be prepared by any means known in the art. Suitable techniques include deglycosylation, trimming, removing the glycosylated amino acid by a non-glycosylated amino acid and/or introducing a tyrosine residue at a non-native position. More specifically, the N-glycoprotein having the exposed tyrosine residue may be prepared by:
(a1) subjecting an N-glycoprotein to deglycosylation by contacting it with an amidase, preferably with PNGase F, to obtain an N-glycoprotein from which the glycan is removed; or
(a2) subjecting an N-glycoprotein to trimming by contacting it with an endoglycosidase, to form an N-glycoprotein having a glycan of structure -GlcNAc(Fuc)_{b}, wherein b is 0 or 1; or
(a3) providing a mutated N-glycoprotein wherein the N-glycosylated amino acid is replaced by a non-glycosylated amino acid.

Deglycosylation of step (a1) is known in the art, and can be performed in any suitable way. Typically, the N-glycoprotein, such as an antibody, is contacted with an amidase which removes the glycan. Thus, step (a1) affords an N-glycoprotein from which the glycan is completely removed, with no remaining monosaccharide moieties. Although any amidase enzyme can be used, beneficial results have been obtained with PNGase F.

Trimming of glycoproteins, as in option (a2), is known in the art, from e.g. Yamamoto, Bitechnol. Lett. 2013, 35, 1733, WO 2007/133855 or WO 2014/065661, which are incorporated herein in their entirety. The trimming of step (a2) can be performed in any suitable way. Typically, the N-glycoprotein, such as an antibody, is contacted with an endoglycosidase. Herein, the endoglycosidase is capable of trimming complex glycans on glycoproteins (such as antibodies) at the core GlcNAc unit, leaving only the core GlcNAc residue on the glycoprotein, which is optionally fucosylated. Depending on the nature of the glycan, a suitable endoglycosidase may be selected. The endoglycosidase is preferably selected from the group consisting of EndoS, EndoA, EndoE, EfEndo18A, EndoF, EndoM, EndoD, EndoH, EndoT and EndoSH and/or a combination thereof, the selection of which depends on the nature of the glycan. EndoSH is described in PCT/EP2017/052792, see Examples 1-3, and SEQ. ID No: 1, which is incorporated by reference herein.

Providing mutated glycoproteins, as in option (a3), is well-known in the art. In the context of the present invention, the glycoprotein may be mutated in any suitable way, typically, by a point mutation. Herein, the N-glycosylated amino acid, typically an asparagine, is replaced by any other amino acid, that is not glycosylated. Any non-glycosylated amino acid is suitable in this context, typically any amino acid except asparagine.

Preferably, a non-mutated N-glycoprotein is used, wherein the glycan is modified according to option (a1) or (a2), most preferably by option (a1).

In an alternative aspect of the present invention, in step (a) a mutant protein is provided, which is in its native form unreactive towards oxidative enzymes capable of oxidizing tyrosine, but is rendered reactive towards such enzyme by providing a mutated form of the protein, wherein a tyrosine residue is introduced at a non-native position in a position of the amino acid sequence of the protein where it is reactive towards oxidative enzymes capable of oxidizing tyrosine. If such a mutant protein is subjected to steps (b), (c) and optionally (d), of the process according to the present invention, it will be conjugated with one or more payloads.

Although the protein may be an N-glycoprotein in the context of the present aspect, it is not necessarily so, since a tyrosine residue is exposed not by modification of the glycan, but by introduction of a tyrosine residue at a specific position. The skilled person is capable of determining the position where the tyrosine residue may be introduced, for example by 3D-modeling of the mutant protein to determine the orientation of the phenolic side chain. The mutation is typically a point mutation.

### Oxidation Step (b)

The exposed tyrosine residue of the N-glycoprotein is subjected to oxidation in step (b), wherein the phenol sidechain of the tyrosine residue is converted into an ortho-quinone moiety. The oxidation is performed by the action of an oxidative enzyme capable of oxidizing tyrosine. Such oxidative enzymes are known in the art, and are preferably selected from tyrosinases, phenol oxidases and polyphenol oxidases. The oxidation of tyrosine residues is known in the art, but is as yet never performed on tyrosine residues that are blocked by a nearby glycan. The present inventors have for the first time been able to subject such tyrosine residues to oxidation by exposing them.

### Reaction Step (c)

The ortho-quinone moiety that is formed during step (b) can be used as chemical handle for further functionalizing the N-glycoprotein. As such, payloads can be conjugated to the N-glycoprotein, in case the payload is functionalized with a moiety reactive towards an ortho-quinone moiety. In step (c), this reaction or conjugation is carried out. Thus, the N-glycoprotein comprising an ortho-quinone moiety is contacted with a compound that comprises a (hetero)cycloalkene or (hetero)cycloalkyne moiety, which is reactive towards the ortho-quinone moiety in a [4+2]cycloaddition, forming a covalent attachment of the glycoprotein with the compound.

The compound further comprises either (i) a chemical handle, herein also referred to as Q², to further modify the compound with a payload D, or (ii) a payload D. Chemical handle Q² can be employed to introduce a payload in a further step (d) as defined below. As such, a conjugate of the glycoprotein and the payload molecule is afforded. The compound that is covalently attached to the glycoprotein is further defined below, as well as the connecting group that is formed upon the reaction of step (c).

The use of (hetero)cycloalkenes and (hetero)cycloalkynes in metal-free click chemistry, such as the [4+2] cycloaddition of step (c), is well-known in the art (see e.g. from WO 2014/065661 and Nguyen and Prescher, Nature rev. 2020, doi: 10.1038/s41570-020-0205-0, both incorporated by reference). These cycloadditions may be strain-promoted, which is also well-known in the art (e.g. a strain-promoted alkyne-azide cycloaddition, SPAAC). In a preferred embodiment, the reaction is a metal-free strain-promoted cycloaddition.

In a preferred embodiment, steps (b) and (c) are performed in a single pot, wherein the N-glycoprotein is contacted simultaneously with the oxidative enzyme and the alkene or alkyne compound.

### Optional Step (d)

In case the compound that is used in step (c) comprises chemical handle Q², it is preferred that the process according to the present invention includes a step (d), wherein the chemical handle obtained in step (c) is subjected to a conjugation reaction with a payload having structure F²-D, wherein F² is reactive towards the chemical handle. Conjugation reactions between two compatible reactive groups, here Q² and F², are well-known in the art, and within the context of the present invention, and conjugation method can be employed.

Care should be taken that the presence of chemical handle Q² does not interfere with the reaction of step (c). So, it is preferred that Q² is not reactive towards ortho-quinone moieties, or the reactivity of Q² towards ortho-quinone moieties is lower than the reactivity of Q¹ towards ortho-quinone moieties, such that in step (c) only Q¹ will react. The product of step (c) is then a glycoprotein modified with a chemical handle Q², which is available for further reaction in step (d). It is also preferred that Q² is not reactive towards Q¹, to avoid polymerization of the compound. In other words, Q² is compatible with Q¹.

In a preferred embodiment, the conjugation reaction between Q² and F² is of the same kind as the conjugation reaction between Q¹ and the ortho-quinone moiety. Thus, preferably the conjugation reaction between Q² and F² is a cycloaddition, preferably a 1,3-dipolar cycloaddition or a [4+2] cycloaddition. The cycloaddition of step (d) is preferably a metal-free strain-promoted cycloaddition. Preferred options for Q² are the same as those for Q¹ defined below, and the skilled person is capable of determining which combination of Q¹ and Q² is suitable such that Q¹ is more reactive then Q² during step (c).

A typical [4+2] cycloaddition is the (hetero)-Diels-Alder reaction, wherein Q² is a diene or a dienophile. As appreciated by the skilled person, the term “diene” in the context of the Diels-Alder reaction refers to 1,3-(hetero)dienes, and includes conjugated dienes (R₂C═CR—CR═CR₂), imines (e.g. R₂C═CR—N═CR₂ or R₂C═CR—CR═NR, R₂C═N—N═CR₂) and carbonyls (e.g. R₂C═CR—CR═O or O═CR—CR═O). Hetero-Diels-Alder reactions with N- and O-containing dienes are known in the art. Any diene known in the art to be suitable for [4+2] cycloadditions may be used as reactive group Q². Preferred dienes include tetrazines, 1,2-quinones and triazines. Although any dienophile known in the art to be suitable for [4+2] cycloadditions may be used as reactive group Q², the dienophile is preferably an alkene or alkyne group as described above, most preferably an alkyne group. For conjugation via a [4+2] cycloaddition, it is preferred that Q² is a dienophile (and F² is a diene), more preferably Q² is or comprises an alkynyl group.

For a 1,3-dipolar cycloaddition, Q² is a 1,3-dipole or a dipolarophile. Any 1,3-dipole known in the art to be suitable for 1,3-dipolar cycloadditions may be used as reactive group Q². Preferred 1,3-dipoles include azido groups, nitrone groups, nitrile oxide groups, nitrile imine groups and diazo groups. Although any dipolarophile known in the art to be suitable for 1,3-dipolar cycloadditions may be used as reactive groups Q², the dipolarophile is preferably an alkene or alkyne group, most preferably an alkyne group. For conjugation via a 1,3-dipolar cycloaddition, it is preferred that Q² is a dipolarophile (and F² is a 1,3-dipole), more preferably Q² is or comprises an alkynyl group.

Thus, in a preferred embodiment, Q² is selected from dipolarophiles and dienophiles.

The skilled person also capable to determine which combination of Q² and F² is suitable for a proper conjugation reaction. Preferred options for F² are selected from an azide, tetrazine, triazine, nitrone, nitrile oxide, nitrile imine, diazo compound, ortho-quinone, dioxothiophene and sydnone, preferably F² is an azide moiety. Further preferred options for F² are provided below.

### The Compound

The compound that is reacted in step (c) comprises a (hetero)cycloalkene or (hetero)cycloalkyne moiety and (i) a chemical handle to further modify the compound with a payload, or (ii) a payload. Typically, the compound has structure (3a) or (3b):

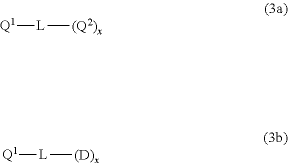

Herein:
Q¹ is a (hetero)cycloalkene or (hetero)cycloalkyne moiety;
L is a linker;
x is an integer in the range of 1-4;
Q² is a chemical handle that is reactive towards an appropriately functionalized payload;
D is a payload.

### Chemical Handle Q¹

Q¹ serves as chemical handle for the connection to the ortho-quinone moiety. In other words, Q¹ is reactive towards the ortho-quinone moiety in a [4+2] cycloaddition. Q¹ is a cyclic (hetero)alkene or a cyclic (hetero)alkyne moiety, most preferably Q is a cyclic (hetero)alkyne moiety.

In an especially preferred embodiment, Q¹ comprises a cyclic (hetero)alkyne moiety. The alkynyl group may also be referred to as a (hetero)cycloalkynyl group, i.e. a heterocycloalkynyl group or a cycloalkynyl group, wherein the (hetero)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Herein, the (hetero)cycloalkynes may optionally be substituted. Preferably, the (hetero)cycloalkynyl group is an optionally substituted (hetero)cycloheptynyl group or an optionally substituted (hetero)cyclooctynyl group. Most preferably, the (hetero)cycloalkynyl group is a (hetero)cyclooctynyl group, wherein the (hetero)cyclooctynyl group is optionally substituted.

In an especially preferred embodiment, Q¹ comprises an (hetero)cycloalkynyl group and is according to structure (Q1):

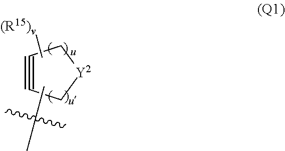

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S) is a cationic sulphur atom counterbalanced by B⁽⁻⁾, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5. Typically, v=(u+u′)×2 or [(u+u′)×2]−1. In a preferred embodiment, v=8, 9 or 10, more preferably v=9 or 10, most preferably v=10.

In a preferred embodiment, Q¹ is selected from the group consisting of (Q2)-(Q20) depicted here below.

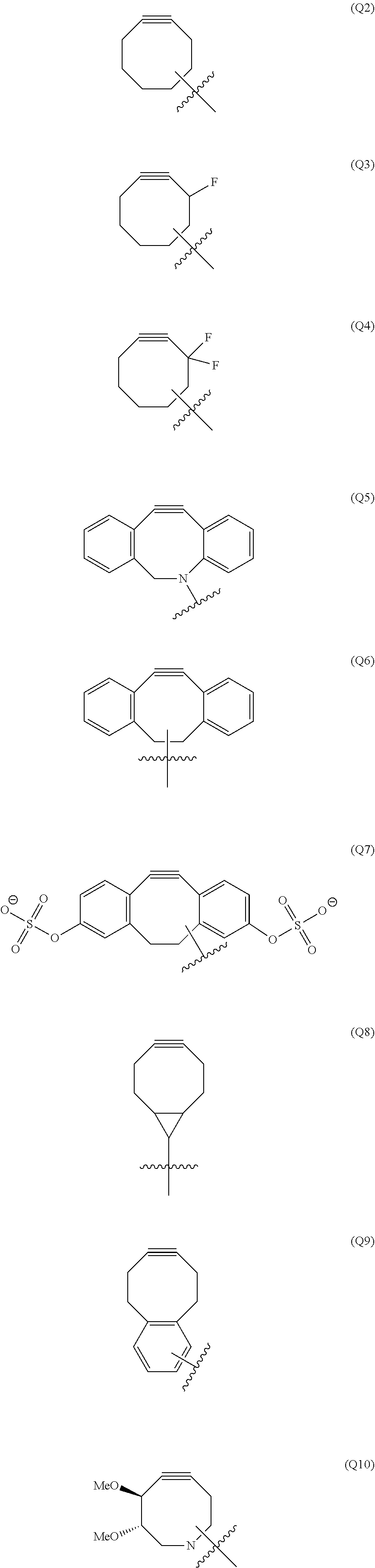

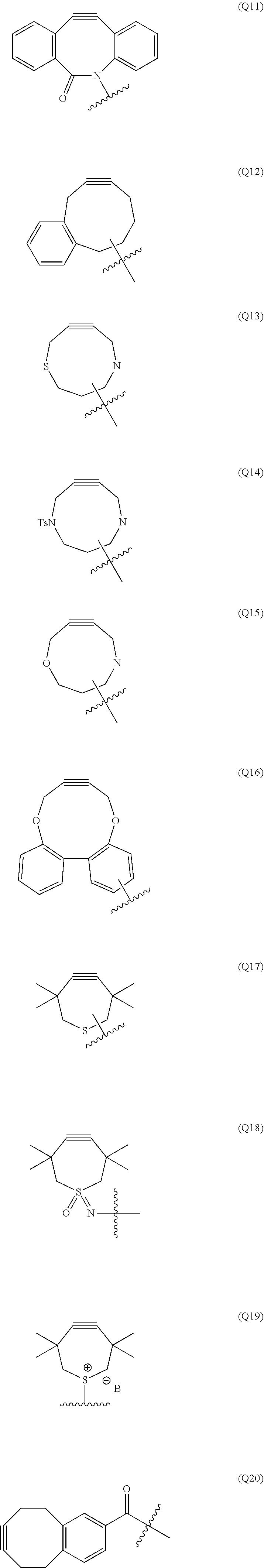

Herein, the connection to L, depicted with the wavy bond, may be to any available carbon or nitrogen atom of Q¹. The nitrogen atom of (Q10), (Q13), (Q14) and (Q15) may bear the connection to L, or may contain a hydrogen atom or be optionally functionalized. B⁽⁻⁾ is an anion, which is preferably selected from ⁽⁻⁾OTf, Cl⁽⁻⁾, Br⁽⁻⁾ or I⁽⁻⁾, most preferably B) is ⁽⁻⁾OTf. In the conjugation reaction, B⁽⁻⁾ does not need to be a pharmaceutically acceptable anion, since B⁽⁻⁾ will exchange with the anions present in the reaction mixture anyway. In case (Q19) is used for Q¹, the negatively charged counter-ion is preferably pharmaceutically acceptable upon isolation of the conjugate according to the invention, such that the conjugate is readily useable as medicament.

In a further preferred embodiment, Q¹ is selected from the group consisting of (Q21)-(Q38) depicted here below.

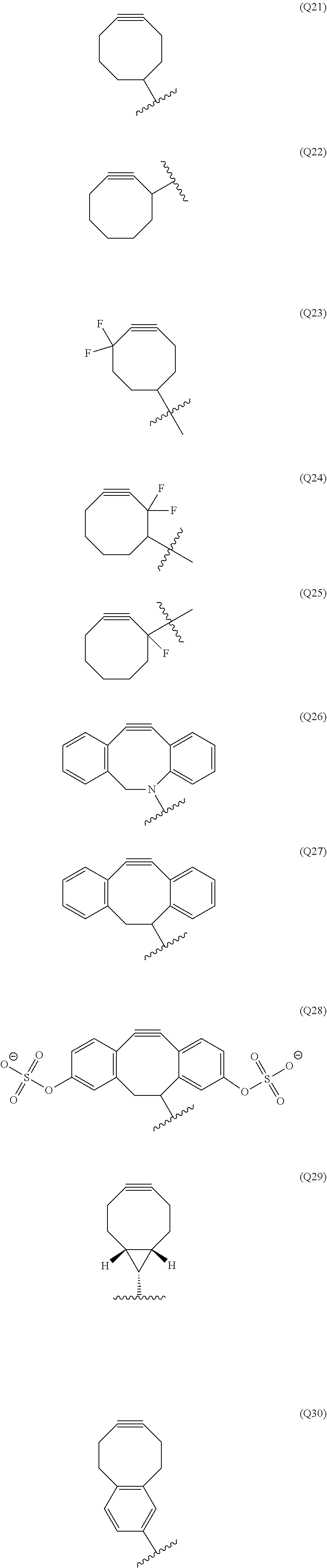

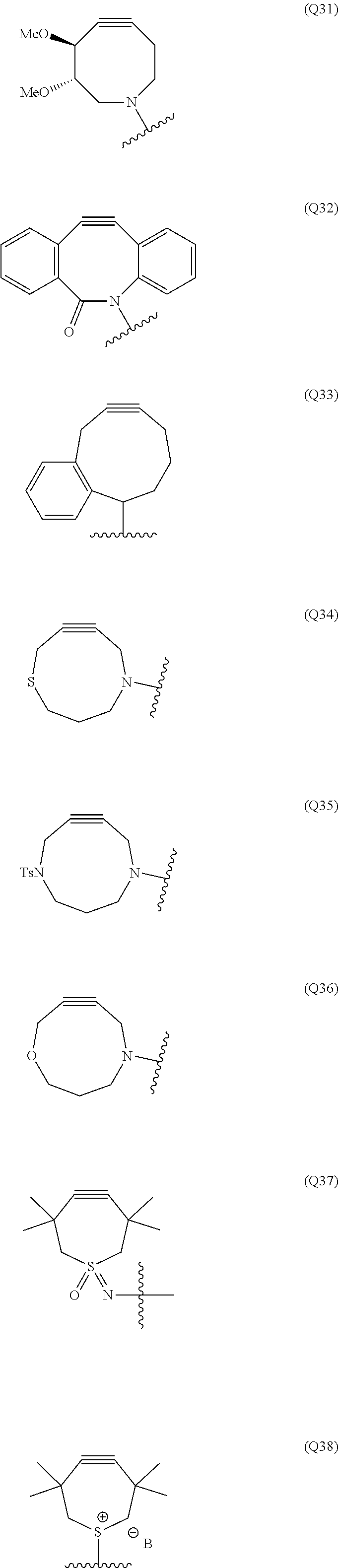

In structure (Q38), B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf.

In a preferred embodiment, Q¹ comprises a (hetero)cyclooctyne moiety according to structure (Q8), more preferably according to (Q29), also referred to as a bicyclo[6.1.0]non-4-yn-9-yl] group (BCN group), which is optionally substituted. In the context of the present embodiment, Q¹ preferably is a (hetero)cyclooctyne moiety according to structure (Q39) as shown below, wherein V is (CH₂)_{I} and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Q39), I is most preferably 1. Most preferably, Q¹ is according to structure (Q42), defined further below.

In an alternative preferred embodiment, Q¹ comprises a (hetero)cyclooctyne moiety according to structure (Q26), (Q27) or (Q28), also referred to as a DIBO, DIBAC, DBCO or ADIBO group, which are optionally substituted. In the context of the present embodiment, Q¹ preferably is a (hetero)cyclooctyne moiety according to structure (Q40) or (Q41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Q40) are optionally O-sulfonylated at one or more positions, whereas the rings of (Q41) may be halogenated at one or more positions. Most preferably, Q¹ is according to structure (Q43), defined further below.

In an alternative preferred embodiment, Q¹ comprises a heterocycloheptynyl group and is according to structure (Q37).

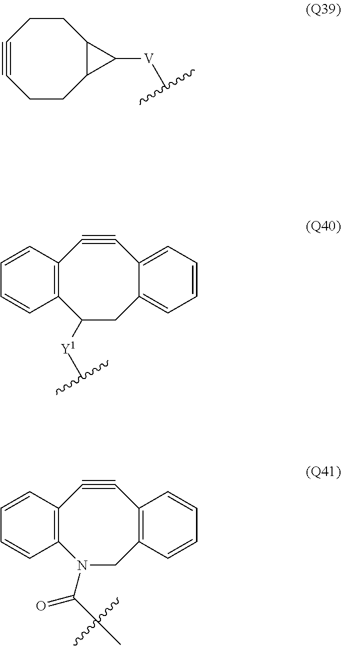

In an especially preferred embodiment, Q¹ comprises a cyclooctynyl group and is according to structure (Q42):

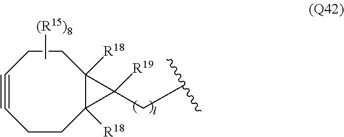

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q¹ or D connected via a spacer moiety; and
I is an integer in the range 0 to 10.

In a preferred embodiment of the reactive group according to structure (Q42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Q42), R¹⁸ is independently selected from the group consisting of hydrogen, C₁-C₆ alkyl groups, most preferably both R¹⁸ are H. In a preferred embodiment of the reactive group according to structure (Q42), R¹⁹ is H. In a preferred embodiment of the reactive group according to structure (Q42), I is 0 or 1, more preferably I is 1.

In an especially preferred embodiment, Q¹ comprises a (hetero)cyclooctynyl group and is according to structure (Q43):

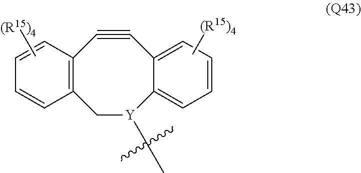

Herein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Q43), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (Q43), Y is N or CH, more preferably Y═N.

In an alternative preferred embodiment, Q¹ comprises a cyclic alkene moiety. The alkenyl group Q¹ may also be referred to as a (hetero)cycloalkenyl group, i.e. a heterocycloalkenyl group or a cycloalkenyl group, preferably a cycloalkenyl group, wherein the (hetero)cycloalkenyl group is optionally substituted. Preferably, the (hetero)cycloalkenyl group is a (hetero)cyclopropenyl group, a (hetero)cyclobutenyl group, a norbornene group, a norbornadiene group, a trans-(hetero)cycloheptenyl group, a trans-(hetero)cyclooctenyl group, a trans-(hetero)cyclononenyl group or a trans-(hetero)cyclodecenyl group, which may all optionally be substituted. Especially preferred are (hetero)cyclopropenyl groups, trans-(hetero)cycloheptenyl group or trans-(hetero)cyclooctenyl groups, wherein the (hetero)cyclopropenyl group, the trans-(hetero)cycloheptenyl group or the trans-(hetero)cyclooctenyl group is optionally substituted. Preferably, Q¹ comprises a cyclopropenyl moiety according to structure (Q44), a hetereocyclobutene moiety according to structure (Q45), a norbornene or norbornadiene group according to structure (Q46), a trans-(hetero)cycloheptenyl moiety according to structure (Q47) or a trans-(hetero)cyclooctenyl moiety according to structure (Q48). Herein, Y³ is selected from C(R²⁴)₂, NR²⁴ or O, wherein each R²⁴ is individually hydrogen, C₁-C₆ alkyl or is connected to L, optionally via a spacer, and the bond labelled [symbol] is a single or double bond. In a further preferred embodiment, the cyclopropenyl group is according to structure (Q49). In another preferred embodiment, the trans-(hetero)cycloheptene group is according to structure (Q50) or (Q51). In another preferred embodiment, the trans-(hetero)cyclooctene group is according to structure (Q52), (Q53), (Q54), (Q55) or (Q56).

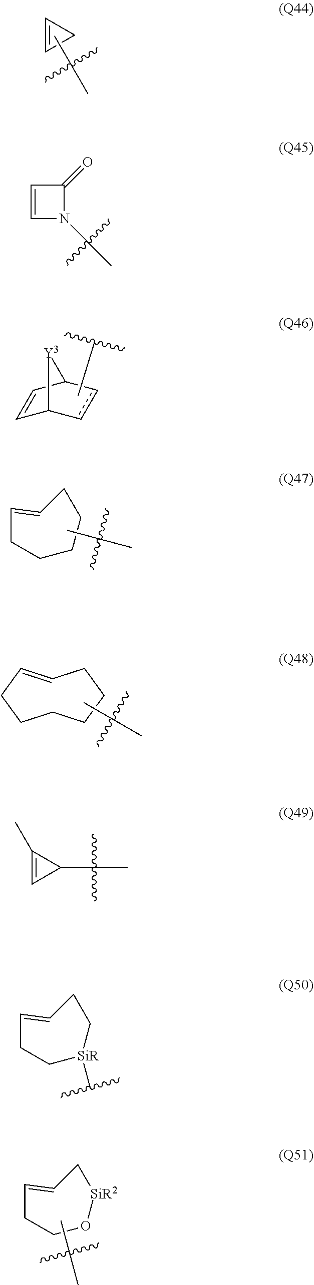

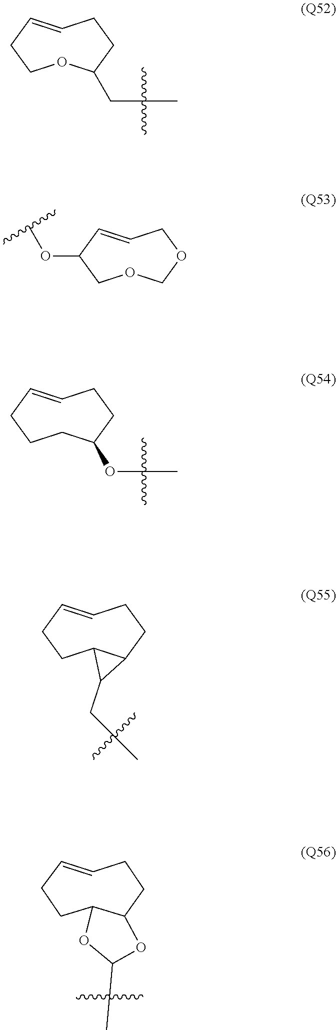

Herein, the R group(s) on Si in (Q50) and (Q51) are typically alkyl or aryl, preferably C₁-C₆ alkyl.

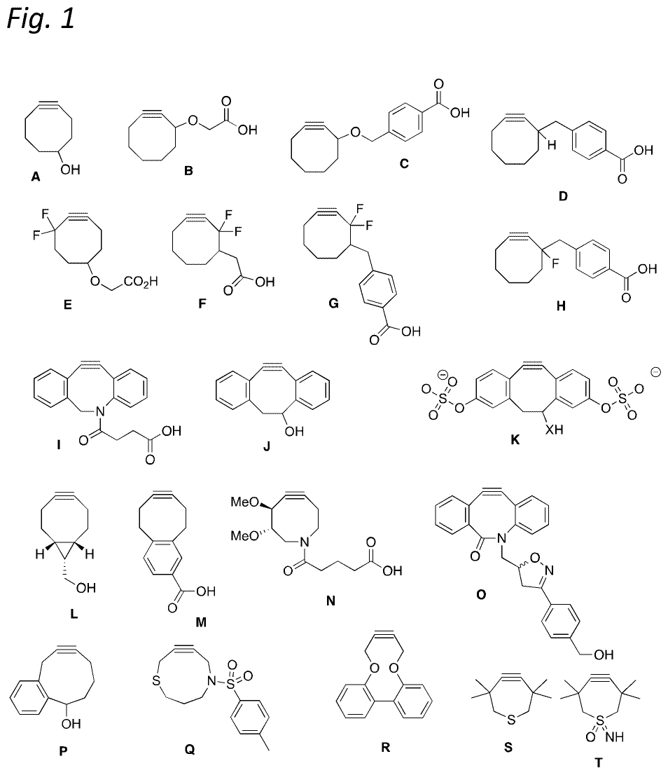

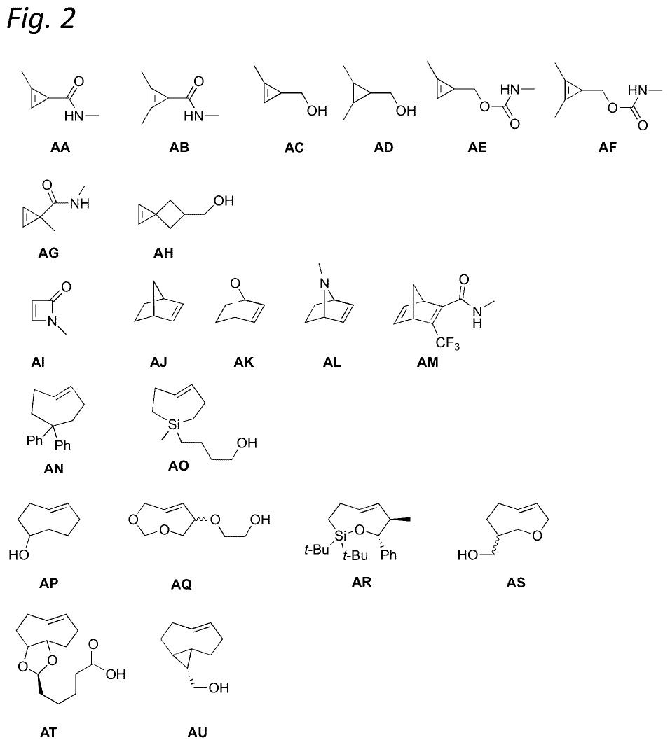

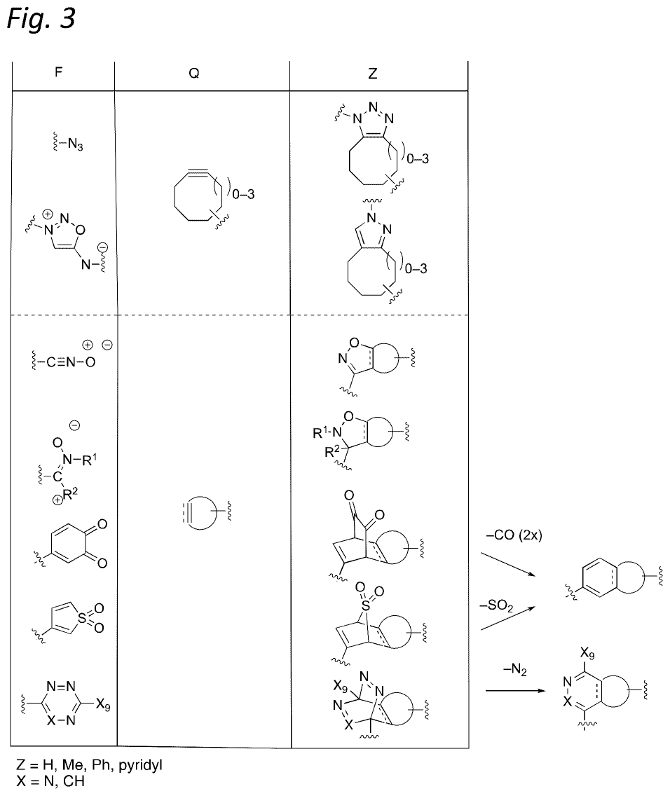

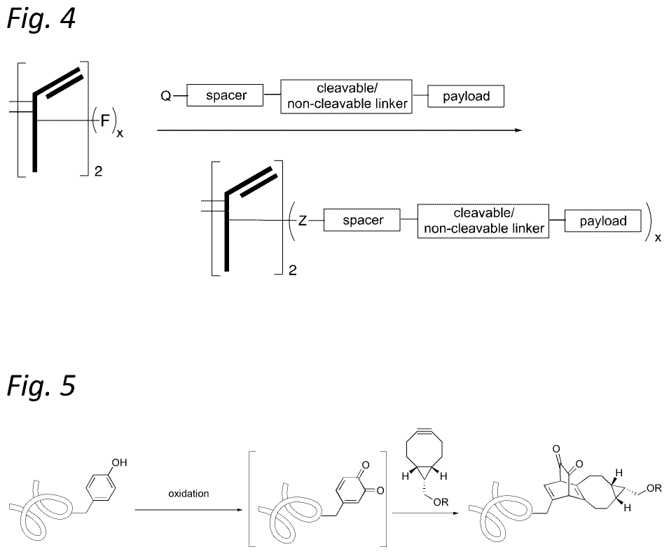

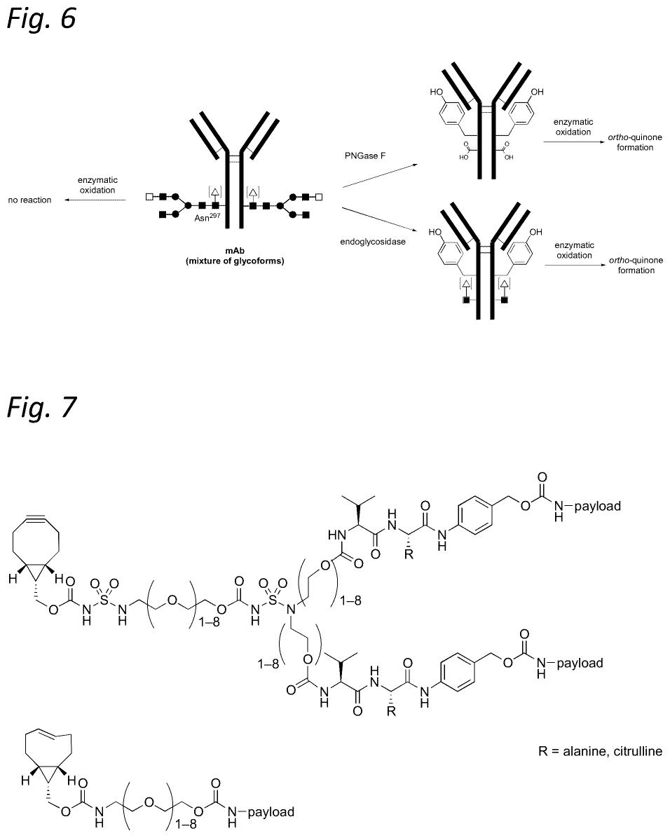

In an alternative preferred embodiment, Q¹ is selected from the structures depicted in FIGS. 1 and 2.

Q² is a chemical handle that is reactive towards an appropriately functionalized payload. The reactivity of Q² is further defined above, in the context of step (d). The appropriately functionalized payload may also be referred to as F²-D or F²-L²-(D)ₓ, wherein F² is reactive towards the chemical handle Q², L² is a linker and x is an integer in the range of 1-4, preferably 1 or 2. In a preferred embodiment, Q² is selected from the same group as Q¹, but is less reactive towards ortho-quinone moieties. In an especially preferred embodiment, Q¹ is a (hetero)cyclooctynyl moiety and Q² is a (hetero)cyclooctenyl moiety. An especially preferred combination is Q¹ being according to structure (Q42) and Q¹ being according to structure (Q48).

### Linker L

Linkers, also referred to as linking units, are well known in the art and any suitable linker may be used. In the compound of structure (3a) or (3b), linker L connects chemical handle Q¹ with chemical handle Q² or payload D. After the reaction of step (c), linker L connects connecting group Z¹ with chemical handle Q² or payload D. Linker L² connects reactive moiety F² with payload D. The linker may be a cleavable or non-cleavable linker. The linker may contain one or more branch-points for attachment of multiple payloads D or multiple chemical handles Q² to a single (hetero)cycloalkene or (hetero)cycloalkyne moiety Q¹. The further definition of the linker here below equally applies to linker L and linker L².

The linker may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR¹², wherein y is 0, 1 or 2, preferably y=2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. The linker may contain (poly)ethylene glycoldiamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z-diaminoalkanes wherein z is the number of carbon atoms in the alkane, and may for example range from 2-25.

In a preferred embodiment, linker L comprises a sulfamide group, preferably a sulfamide group according to structure (L1):

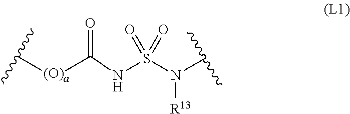

The wavy lines represent the connection to the remainder of the compound or conjugate, typically to Q¹ or Z¹ and to Q² or D, optionally via a spacer. Preferably, the (O)ₐC(O) moiety is connected to Q¹ or Z¹ and the NR¹³ moiety to Q² or D.

In structure (L1), a=0 or 1, preferably a=1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of Q² or D connected to N via a spacer moiety, preferably Sp² as defined here below.

In a preferred embodiment, R¹³ is hydrogen or a C₁-C₂₀ alkyl group, more preferably R¹³ is hydrogen or a C₁-C₁₆ alkyl group, even more preferably R¹³ is hydrogen or a C₁-C₁₀ alkyl group, wherein the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In a preferred embodiment, R¹³ is hydrogen. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an —OH group, preferably a terminal —OH group. In this embodiment it is further preferred that R¹³ is a (poly)ethylene glycol chain comprising a terminal —OH group. In another preferred embodiment, R¹³ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R¹³ is hydrogen or methyl, and most preferably R¹³ is hydrogen.

In a preferred embodiment, the linker is according to structure (L2):

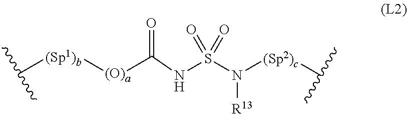

Herein, a, R¹³ and the wavy lines are as defined above, Sp¹ and Sp² are independently spacer moieties and b and c are independently 0 or 1. Preferably, b=0 or 1 and c=1, more preferably b=0 and c=1. In one embodiment, spacers Sp¹ and Sp² are independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, spacer moieties Sp¹ and Sp², if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR²⁰, preferably O, wherein R²⁰ is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR²⁰, preferably O and/or S—S, wherein R²⁰ is independently selected from the group consisting of hydrogen and C₁-C₄ alkyl groups, preferably hydrogen or methyl.

Another class of suitable linkers comprises cleavable linkers. Cleavable linkers are well known in the art. For example Shabat et al., Soft Matter 2012, 6, 1073, incorporated by reference herein, discloses cleavable linkers comprising self-immolative moieties that are released upon a biological trigger, e.g. an enzymatic cleavage or an oxidation event. Some examples of suitable cleavable linkers are peptide-linkers that are cleaved upon specific recognition by a protease, e.g. cathepsin, plasmin or metalloproteases, or glycoside-based linkers that are cleaved upon specific recognition by a glycosidase, e.g. glucoronidase, or nitroaromatics that are reduced in oxygen-poor, hypoxic areas.

Linker L may further contain a peptide spacer as known in the art, preferably a dipeptide or tripeptide spacer as known in the art, preferably a dipeptide spacer. Although any dipeptide or tripeptide spacer may be used, preferably the peptide spacer is selected from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, more preferably Val-Cit, Val-Ala, Ala-Ala-Asn. In one embodiment, the peptide spacer is Val-Cit. In one embodiment, the peptide spacer is Val-Ala. The peptide spacer may also be attached to the payload, wherein the amino end of the peptide spacer is conveniently used as amine group in the method according to the first aspect of the invention.

In a preferred embodiment, the peptide spacer is represented by general structure (L3):

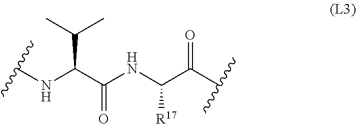

Herein, R¹⁷═CH₃ (Val) or CH₂CH₂CH₂NHC(O)NH₂ (Cit). The wavy lines indicate the connection to the remainder of the molecule, preferably the peptide spacer according to structure (L3) is connected via NH to Q¹ or Z¹, typically via a spacer, and via C(O) to Q² or D, typically via a spacer.

Linker L may further contain a self-cleavable spacer, also referred to as self-immolative spacer. The self-cleavable spacer may also be attached to the payload. Preferably, the self-cleavable spacer is para-aminobenzyloxycarbonyl (PABC) derivative, more preferably a PABC derivative according to structure (L4).

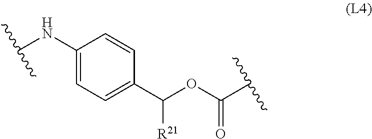

Herein, the wavy lines indicate the connection to the remainder of the molecule. Typically, the PABC derivative is connected via NH to Q¹ or Z¹, typically via a spacer, and via OC(O) to Q² or D, typically via a spacer.

R²¹ is H, R²² or C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups. Preferably, R²² is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R²¹ is H or C(O)R²², wherein R²²=4-methyl-piperazine or morpholine. Most preferably, R²¹ is H.

### Payload D

Linker L connects the (hetero)cycloalkane or (hetero)cycloalkyne moiety Q¹ with chemical handle Q² or payload D. Payload D may also be introduced in step (d). Payload molecules are well-known in the art, especially in the field of antibody-drug conjugates, as the moiety that is covalently attached to the antibody and that is released therefrom upon uptake of the conjugate and/or cleavage of the linker. In a preferred embodiment, the payload is selected from the group consisting of an active substance, a reporter molecule, a polymer, a solid surface, a hydrogel, a nanoparticle, a microparticle and a biomolecule. Especially preferred payloads are active substances and reporter molecules, in particular active substances.

The term “active substance” herein relates to a pharmacological and/or biological substance, i.e. a substance that is biologically and/or pharmaceutically active, for example a drug, a prodrug, a cytotoxin, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine. When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterial agents, peptides and oligonucleotides. Examples of cytotoxins include colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, auristatins, enediynes, pyrrolobenzodiazepines (PBDs) or indolinobenzodiazepine dimers (IGN) or PNU159,682 and derivatives thereof. Preferred payloads are selected from MMAE, MMAF, exatecan, SN-38, DXd, maytansinoids, calicheamicin, PNU159,685 and PBD dimers. Especially preferred payloads are PBD, SN-38, MMAE, exatecan or DXd. In one embodiment, the payload is MMAE. In one embodiment, the payload is exatecan or DXd. In one embodiment, the payload is SN-38. In one embodiment, the payload is MMAE. In one embodiment, the payload is a PDB dimer.

The term “reporter molecule” herein refers to a molecule whose presence is readily detected, for example a diagnostic agent, a dye, a fluorophore, a radioactive isotope label, a contrast agent, a magnetic resonance imaging agent or a mass label.

A wide variety of fluorophores, also referred to as fluorescent probes, is known to a person skilled in the art. Several fluorophores are described in more detail in e.g. G. T. Hermanson, “Bioconjugate Techniques”, Elsevier, 3^{rd} Ed. 2013, Chapter 10: “Fluorescent probes”, p. 395-463, incorporated by reference. Examples of a fluorophore include all kinds of Alexa Fluor (e.g. Alexa Fluor 555), cyanine dyes (e.g. Cy3 or Cy5) and cyanine dye derivatives, coumarin derivatives, fluorescein and fluorescein derivatives, rhodamine and rhodamine derivatives, boron dipyrromethene derivatives, pyrene derivatives, naphthalimide derivatives, phycobiliprotein derivatives (e.g. allophycocyanin), chromomycin, lanthanide chelates and quantum dot nanocrystals.

Examples of a radioactive isotope label include ^{99m}Tc, ¹¹¹In, ^{114m}In, ¹¹⁵In, ¹⁸F, ¹⁴C, ⁶⁴Cu, ¹³¹I, ¹²⁵I, ¹²³I, ²¹²Bi, ⁸⁸Y, ⁹⁰Y, ⁶⁷Cu, ¹⁸⁶Rh, ¹⁸⁸Rh, ⁶⁶Ga, ⁶⁷Ga and ¹⁰B, which is optionally connected via a chelating moiety such as e.g. DTPA (diethylenetriaminepentaacetic anhydride), DOTA (1,4,7,10-tetraazacyclododecane-N,N′,N,N′″tetraacetic acid), NOTA (1,4,7-triazacyclononane N,N,N″-triacetic acid), TETA (1,4,8,11-tetraazacyclotetradecane-N,N′,N,N″′-tetraacetic acid), DTTA (N¹-(p-isothiocyanatobenzyl)-diethylenetriamine-N,N²,N³,N³-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide). Isotopic labelling techniques are known to a person skilled in the art, and are described in more detail in e.g. G. T. Hermanson, “Bioconjugate Techniques”, Elsevier, 3^{rd} Ed. 2013, Chapter 12: “Isotopic labelling techniques”, p. 507-534, incorporated by reference.

Polymers suitable for use as a payload D in the compound according to the invention are known to a person skilled in the art, and several examples are described in more detail in e.g. G. T. Hermanson, “Bioconjugate Techniques”, Elsevier, 3^{rd} Ed. 2013, Chapter 18: “PEGylation and synthetic polymer modification”, p. 787-838, incorporated by reference. When payload D is a polymer, payload D is preferably independently selected from the group consisting of a poly(ethyleneglycol) (PEG), a polyethylene oxide (PEO), a polypropylene glycol (PPG), a polypropylene oxide (PPO), a 1,q-diaminoalkane polymer (wherein q is the number of carbon atoms in the alkane, and preferably q is an integer in the range of 2 to 200, preferably 2 to 10), a (poly)ethylene glycol diamine (e.g. 1,8-diamino-3,6-dioxaoctane and equivalents comprising longer ethylene glycol chains), a polysaccharide (e.g. dextran), a poly(amino acid) (e.g. a poly(L-lysine)) and a poly(vinyl alcohol).

Solid surfaces suitable for use as a payload D are known to a person skilled in the art. A solid surface is for example a functional surface (e.g. a surface of a nanomaterial, a carbon nanotube, a fullerene or a virus capsid), a metal surface (e.g. a titanium, gold, silver, copper, nickel, tin, rhodium orzinc surface), a metal alloy surface (wherein the alloy is from e.g. aluminum, bismuth, chromium, cobalt, copper, gallium, gold, indium, iron, lead, magnesium, mercury, nickel, potassium, plutonium, rhodium, scandium, silver, sodium, titanium, tin, uranium, zinc and/or zirconium), a polymer surface (wherein the polymer is e.g. polystyrene, polyvinylchloride, polyethylene, polypropylene, poly(dimethylsiloxane) or polymethylmethacrylate, polyacrylamide), a glass surface, a silicone surface, a chromatography support surface (wherein the chromatography support is e.g. a silica support, an agarose support, a cellulose support or an alumina support), etc. When payload D is a solid surface, it is preferred that D is independently selected from the group consisting of a functional surface or a polymer surface.

Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. When the payload is a hydrogel, it is preferred that the hydrogel is composed of poly(ethylene)glycol (PEG) as the polymeric basis.

Micro- and nanoparticles suitable for use as a payload D are known to a person skilled in the art. A variety of suitable micro- and nanoparticles is described in e.g. G. T. Hermanson, “Bioconjugate Techniques”, Elsevier, 3^{rd} Ed. 2013, Chapter 14: “Microparticles and nanoparticles”, p. 549-587, incorporated by reference. The micro- or nanoparticles may be of any shape, e.g. spheres, rods, tubes, cubes, triangles and cones. Preferably, the micro- or nanoparticles are of a spherical shape. The chemical composition of the micro- and nanoparticles may vary. When payload D is a micro- or a nanoparticle, the micro- or nanoparticle is for example a polymeric micro- or nanoparticle, a silica micro- or nanoparticle or a gold micro- or nanoparticle. When the particle is a polymeric micro- or nanoparticle, the polymer is preferably polystyrene or a copolymer of styrene (e.g. a copolymer of styrene and divinylbenzene, butadiene, acrylate and/or vinyltoluene), polymethylmethacrylate (PMMA), polyvinyltoluene, poly(hydroxyethyl methacrylate (pHEMA) or poly(ethylene glycol dimethacrylate/2-hydroxyethylmetacrylae) [poly(EDGMA/HEMA)]. Optionally, the surface of the micro- or nanoparticles is modified, e.g. with detergents, by graft polymerization of secondary polymers or by covalent attachment of another polymer or of spacer moieties, etc.

Payload D may also be a biomolecule. Biomolecules, and preferred embodiments thereof, are described in more detail below. When payload D is a biomolecule, it is preferred that the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides. In the context of the present invention, cytotoxic payloads are especially preferred. Thus, D is preferably, a cytotoxin, more preferably selected from the group consisting of colchicine, vinca alkaloids, anthracyclines, camptothecins, doxorubicin, daunorubicin, taxanes, calicheamycins, tubulysins, irinotecans, an inhibitory peptide, amanitins, amatoxins, deBouganin, duocarmycins, epothilones, mytomycins, combretastatins, maytansines, auristatins, enediynes, pyrrolobenzodiazepines (PBDs) or indolinobenzodiazepine dimers (IGN) or PNU159,682. In an especially preferred embodiment, D is MMAE or exatecan.

### The Conjugate

A further aspect of the invention concerns the conjugate that is obtainable by the process according to the invention. Alternatively, the conjugate according to the invention is defined as having a structure (1a) or (1b):

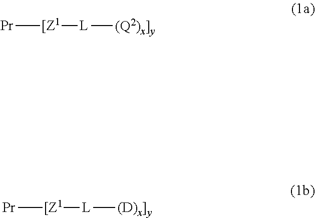

Herein:
Pr is an N-glycoprotein;
Z¹ is a connecting group comprising structure (Za) or (Zb):

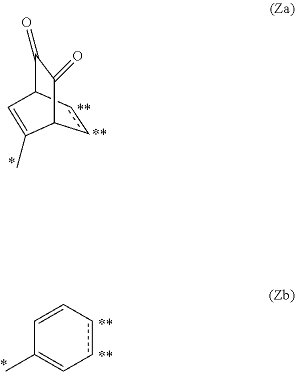

wherein the carbon labelled with * is directly connected to the peptide chain of the antibody at an amino acid located within 10 amino acids of an N-glycosylation site, which has been modified such that the glycoprotein does not contain a glycan longer than two monosaccharide residues within 10 amino acids of the amino acid residue, and both of the carbon atoms labelled with ** are connected to L, and the bond depicted as [symbol] is a single bond or a double bond;
L is a linker;
x is an integer in the range of 1-4;
y is an integer in the range of 1-4;
Q² is a chemical handle that is reactive towards an appropriately functionalized payload;
D is a payload.

The integer y denotes the number of tyrosine residues that are oxidized in step (b) and subsequently used as conjugation site in step (c) and optionally (d). Preferably, y=1, 2 or 4, most preferably y=2 or 4. The integer x denotes the number of chemical handles Q² or payloads D are connected to the linker. The linker may be linear, having only one occurrence of Q² or D connected to it, or may contain one or more branching points to connect up to 4 occurrences of Q² or D to the same connecting group Z¹. Preferably, x is 1 or 2. In case the compound according to structure (3b) is reacted in step (c), and in case the conjugate is according to structure (1b), it is preferred that x is 1 or 2, most preferably x=2. In case the compound according to structure (3a) is reacted in step (c), and in case the conjugate is according to structure (1a), it is preferred that x is 1 or 2, most preferably x=1.

The glycoprotein Pr, linker L, payload D and chemical handle Q² are further defined above, which definitions equally apply to the conjugate according to the present aspect.

### Connecting Group Z¹

A connecting group, also referred to herein as Z¹, is formed upon reaction of step (c). Connecting group Z¹ covalently connects the glycoprotein with the compound as defined above, more in particular with chemical handle Q² or payload D. Connecting group Z¹ comprises structure (Za) or (Zb):

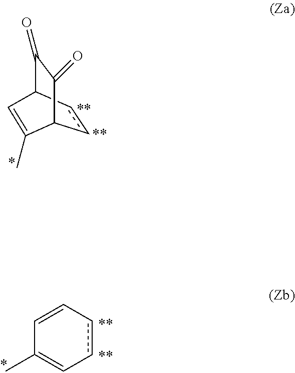

Herein, the carbon labelled with * is directly connected to the peptide chain of the antibody and both carbon atoms labelled with ** are connected to L. The bond depicted as [symbol] is a single bond or a double bond, and:
L is a linker;
x is an integer in the range of 1-4;
Q² is a chemical handle that is reactive towards an appropriately functionalized payload;
D is a payload

Connecting group Z¹ is formed by reaction of the ortho-quinone moiety with the (hetero)cycloalkene moiety, giving a single bond for [symbol] , or the (hetero)cycloalkyne moiety, giving a double bond for [symbol] . As the (hetero)alkene or (hetero)alkyne is present in a cyclic structure, both carbon atoms of the resulting C [symbol] C bond (labelled with **) will also be in a cyclic structure. In other words, both carbon atoms are connected to L via that cyclic structure. The carbon labelled with * originates from the exposed tyrosine residue and corresponds to the CH₂ moiety that connects the phenol moiety to the peptide main chain of the glycoprotein. In the connecting group, the CH₂ moiety labelled with * is thus directly connected to the peptide main chain.

In the [4+2] cycloaddition, the connecting group of structure (Za) is first formed. Depending on the conditions, this connecting group may eliminate two molecules of CO and in situ form the connecting group of structure (Zb). In the context of the present invention, the exact nature of the connecting group is irrelevant, as in any case it serves as a covalent attachment of Q² or D to the glycoprotein.

In one embodiment, Z¹ comprises a (hetero)cycloalkene moiety, i.e. is formed from Q¹ comprising a (hetero)cycloalkyne moiety. In an alternative embodiment, Z¹ comprises a (hetero)cycloalkane moiety, i.e. is formed from Q¹ comprising a (hetero)cycloalkene moiety. In a preferred embodiment, Z¹ has the structure (Z1a) or (Z1b):

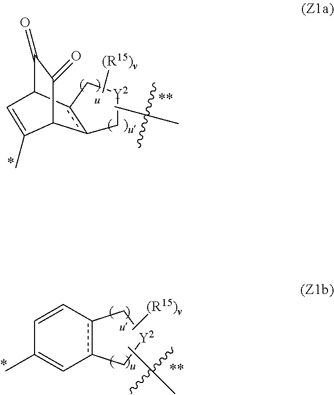

Herein, the carbon labelled with * is directly connected to the peptide chain of the antibody and the bond labelled with ** is connected to L, and the bond depicted as [symbol] is a single bond or a double bond. Furthermore:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S) is a cationic sulphur atom counterbalanced by B3, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 8-16.

In case the bond depicted as [symbol] is a double bond, it is preferred that u+u′=4, 5, 6, 7 or 8.

It is especially preferred that Z¹ comprises a (hetero)cycloalkene moiety, i.e. the bond depicted as [symbol] is a double bond. In a preferred embodiment, Z¹ is selected from the structures (Z2)-(Z20), depicted here below:

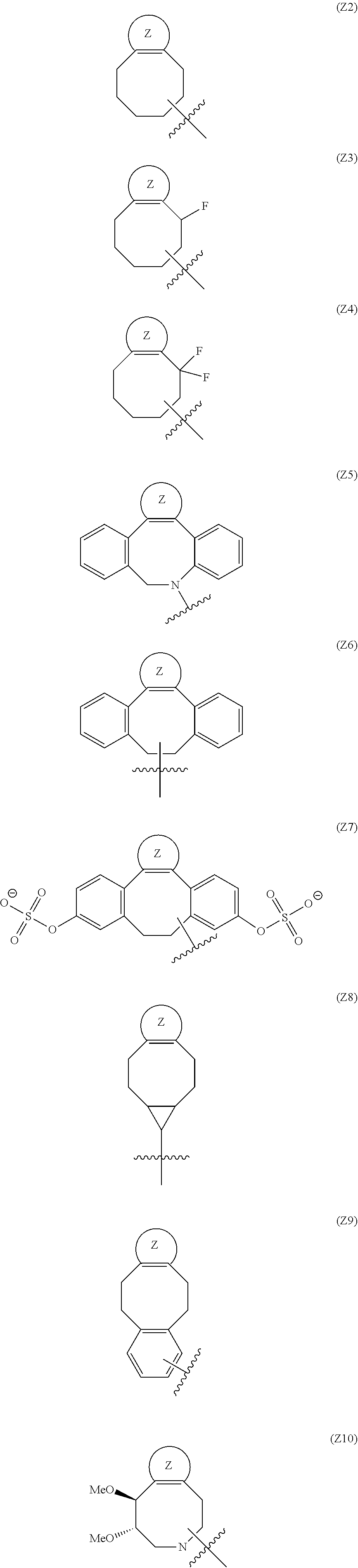

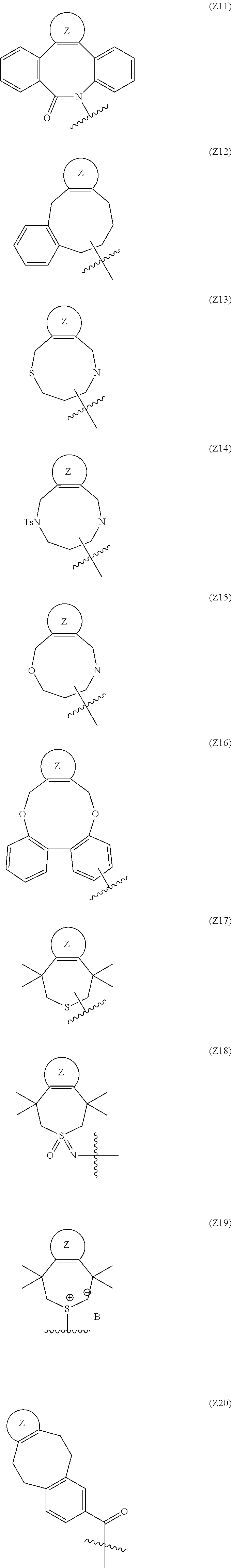

Herein, the connection to L is depicted with the wavy bond. B⁽⁻⁾ is an anion, preferably a pharmaceutically acceptable anion. Ring Z is either of structure (Za) or structure (Zb), wherein the carbon atoms labelled with ** correspond to the two carbon atoms of the (hetero)cycloalkane ring of (Z2)-(Z20) to which ring Z is fused, and the carbon a carbon labelled with * is directly connected to the peptide chain of the antibody. Since the connecting group Z is formed by reaction with a (hetero)cycloalkyne in the context of the present embodiment, the bound depicted above as [symbol] is a double bond.

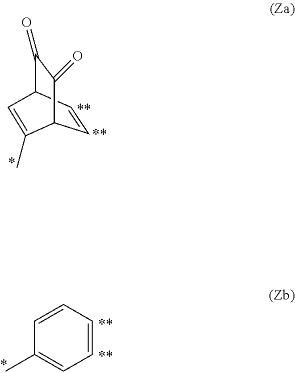

In a further preferred embodiment, Z¹ is selected from the structures (Z21)-(Z38), depicted here below:

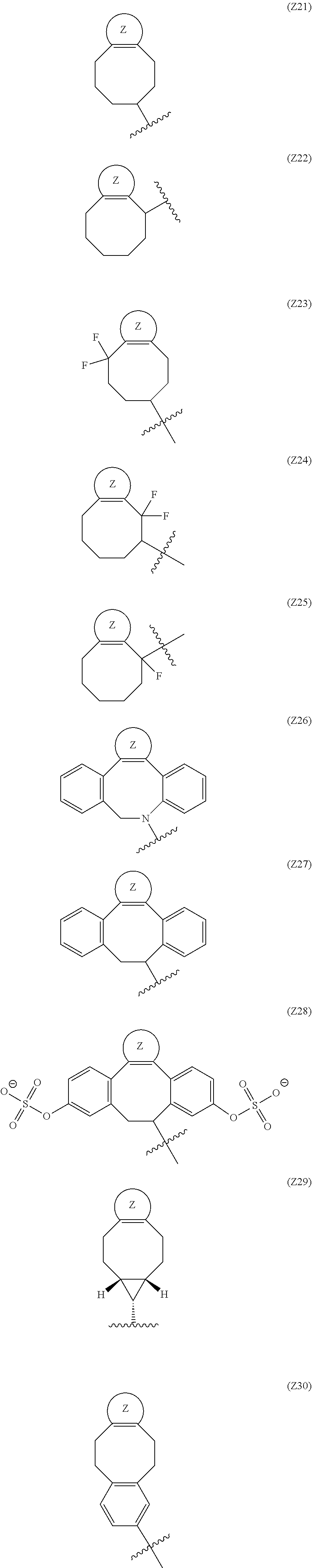

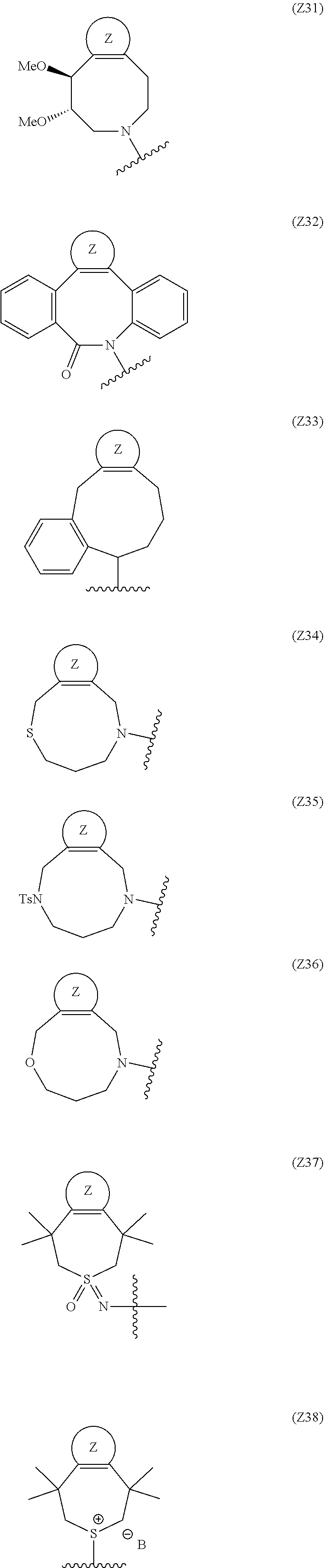

Herein, the connection to L is depicted with the wavy bond. In structure (Z38), B) is an anion, preferably a pharmaceutically acceptable anion. Ring Z is either of structure (Za) or structure (Zb), as defined above.

In a preferred embodiment, Z¹ comprises a (hetero)cyclooctene moiety according to structure (Z8), more preferably according to (Z29), which is optionally substituted. In the context of the present embodiment, Z¹ preferably comprises a (hetero)cyclooctene moiety according to structure (Z39) as shown below, wherein V is (CH₂)_{I} and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Z39), I is most preferably 1. Most preferably, Z¹ is according to structure (Z42), defined further below.

In an alternative preferred embodiment, Z¹ comprises a (hetero)cyclooctene moiety according to structure (Z26), (Z27) or (Z28), which are optionally substituted. In the context of the present embodiment, Z¹ preferably comprises a (hetero)cyclooctene moiety according to structure (Z40) or (Z41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Z40) are optionally O-sulfonylated at one or more positions, whereas the rings of (Z41) may be halogenated at one or more positions. Most preferably, Z¹ is according to structure (Z43), defined further below.

In an alternative preferred embodiment, Z¹ comprises a heterocycloheptenyl group and is according to structure (Z37).

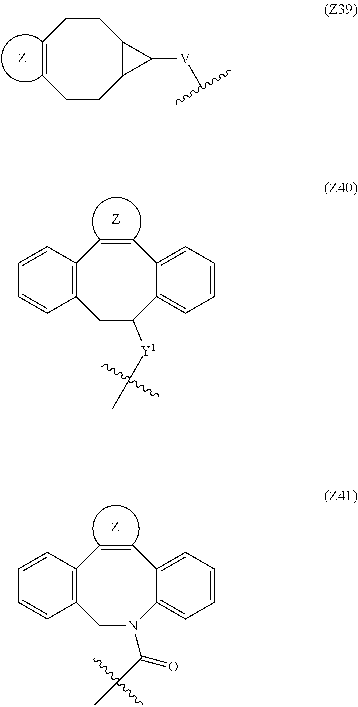

In an especially preferred embodiment, Z¹ comprises a cyclooctynyl group and is according to structure (Z42a) or (Z42b):

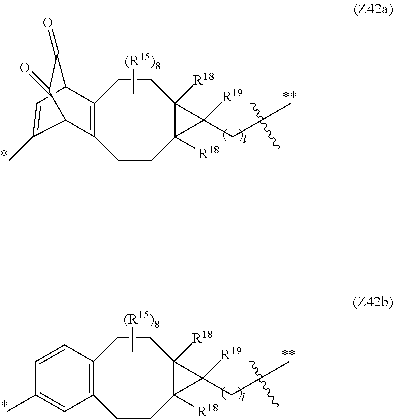

### Herein:

the carbon labelled with * is directly connected to the peptide chain of the antibody and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q¹ or D connected via a spacer moiety; and
I is an integer in the range 0 to 10.

In a preferred embodiment of the reactive group according to structure (Z42a) or (Z42b), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Z42a) or (Z42b), R¹⁸ is independently selected from the group consisting of hydrogen, C₁-C₆ alkyl groups, most preferably both R¹⁸ are H. In a preferred embodiment of the reactive group according to structure (Z42a) or (Z42b), R¹⁹ is H. In a preferred embodiment of the reactive group according to structure (Z42a) or (Z42b), I is 0 or 1, more preferably I is 1.

In an especially preferred embodiment, Q¹ comprises a (hetero)cyclooctynyl group and is according to structure (Z43a) or (Z43b):

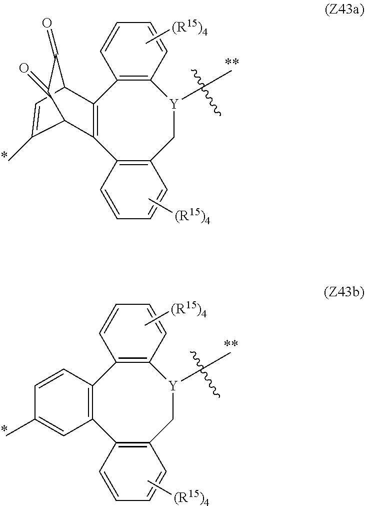

### Herein:

the carbon labelled with * is directly connected to the peptide chain of the antibody and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Z43a) or (Z43b), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (Z43a) or (Z43b), Y is N or CH, more preferably Y═N.

In an alternative preferred embodiment, Z¹ comprises a (hetero)cycloalkane moiety, i.e. the bond depicted as [symbol] is a single bond. The (hetero)cycloalkane group may also be referred to as a heterocycloalkanyl group or a cycloalkanyl group, preferably a cycloalkanyl group, wherein the (hetero)cycloalkanyl group is optionally substituted. Preferably, the (hetero)cycloalkanyl group is a (hetero)cyclopropanyl group, a (hetero)cyclobutanyl group, a norbornane group, a norbornene group, a (hetero)cycloheptanyl group, a (hetero)cyclooctanyl group, a (hetero)cyclononnyl group or a (hetero)cyclodecanyl group, which may all optionally be substituted. Especially preferred are (hetero)cyclopropanyl groups, (hetero)cycloheptanyl group or (hetero)cyclooctanyl groups, wherein the (hetero)cyclopropanyl group, the trans-(hetero)cycloheptanyl group or the (hetero)cyclooctanyl group is optionally substituted. Preferably, Z¹ comprises a cyclopropanyl moiety according to structure (Z44), a hetereocyclobutane moiety according to structure (Z45), a norbornane or norbornene group according to structure (Z46), a (hetero)cycloheptanyl moiety according to structure (Z47) or a (hetero)cyclooctanyl moiety according to structure (Z48). Herein, Y³ is selected from C(R²⁴)₂, NR²⁴ or O, wherein each R²⁴ is individually hydrogen, C₁-C₆ alkyl or is connected to L, optionally via a spacer, and the bond labelled [symbol] is a single or double bond. In a further preferred embodiment, the cyclopropanyl group is according to structure (Z49). In another preferred embodiment, the (hetero)cycloheptane group is according to structure (Z50) or (Z51). In another preferred embodiment, the (hetero)cyclooctane group is according to structure (Z52), (Z53), (Z54), (Z55) or (Z56).

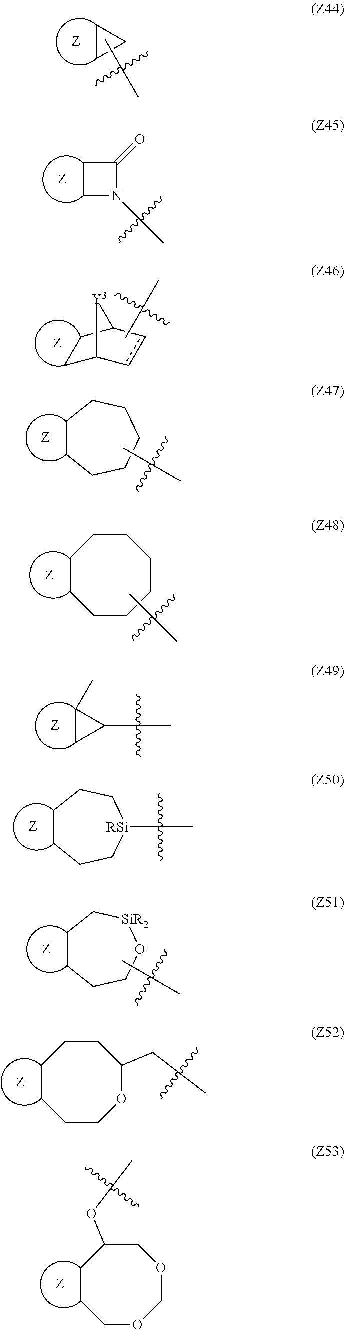

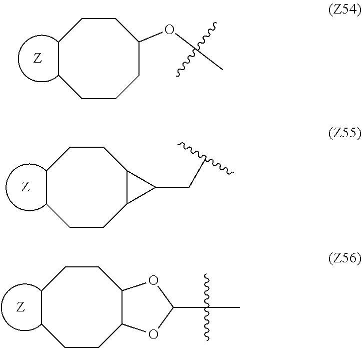

Herein, the R group(s) on Si in (Z50) and (Z51) are typically alkyl or aryl, preferably C₁-C₆ alkyl. Ring Z is either of structure (Za) or structure (Zb), wherein the carbon atoms labelled with ** correspond to the two carbon atoms of the (hetero)cycloalkane ring of (Z44)-(Z56) to which ring Z is fused, and the carbon a carbon labelled with * is directly connected to the peptide chain of the antibody. Since the connecting group Z is formed by reaction with a (hetero)cycloalkene in the context of the present embodiment, the bound depicted above as [symbol] is a single bond.

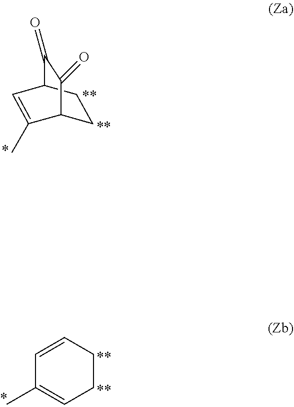

In an alternative preferred embodiment, Z¹ comprises a (hetero)cycloalkane group or a (hetero)cycloalkane group formed by conjugation reaction of the ortho-quinone and a chemical handle selected from the structures depicted in FIGS. 1 and 2.

In an alternative aspect of the present invention, the glycoprotein-conjugate has structure (1a) or (1b), wherein:
Pr is a protein;
Z¹ comprises structure (Za) or (Zb):

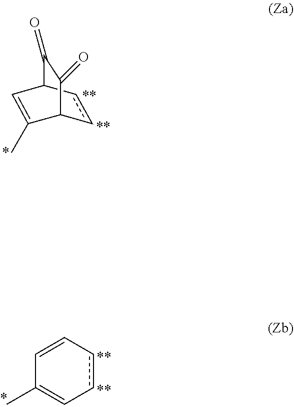

wherein the carbon labelled with * is directly connected to the peptide chain of the glycoprotein at an amino acid which is in the native form of the protein not a tyrosine residue and both of the carbon atoms labelled with ** are connected to L, and the bond depicted as [symbol] is a single bond or a double bond.

Z¹, L, x, y, Q² and D are as further defined elsewhere.

The protein is a mutant protein, which is in its native form unreactive towards oxidative enzymes capable of oxidizing tyrosine, but is rendered reactive towards such enzyme by providing a mutated form of the protein, wherein a tyrosine residue is introduced at a non-native position in a position of the amino acid sequence of the protein where it is reactive towards oxidative enzymes capable of oxidizing tyrosine. Thus, the amino acid to which the connecting group Z¹ is connected is located at a position where a tyrosine residue is reactive towards oxidative enzymes capable of oxidizing tyrosine. Typically, the protein has undergone a point mutation to introduce the tyrosine residue at the desired location.

Also part of the present invention is a process for preparing a conjugate according to structure (1b), comprising reacting a conjugate according to structure (1a) with a with a payload having structure D-F² or D-L²-(F²)ₓ, wherein F² is reactive towards Q² in a conjugation reaction. Herein, L² is a linker and x an integer in the range of 1-4. In the context of the present invention, this payload may also be referred to as functionalized payload. This conjugation reaction corresponds to step (d) defined above, and everything defined for step (d) equally applies to the process according to the present aspect, and vice versa.

The functionalized payload is contacted with the conjugate according to structure (1a). Herein, F² is reactive towards Q² in a conjugation reaction, preferably a cycloaddition. Preferably, F² is reactive towards a (hetero)cycloalkene and/or a (hetero)cycloalkyne, and is typically selected from the group consisting of azide, tetrazine, triazine, nitrone, nitrile oxide, nitrile imine, diazo compound, ortho-quinone, dioxothiophene and sydnone. Preferred structures for the reactive group are structures (F1)-(F10) depicted here below.

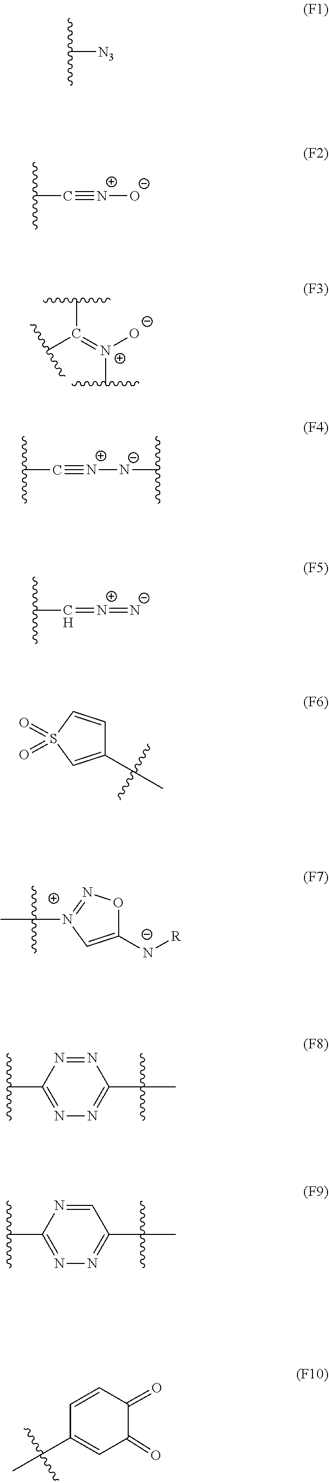

Herein, the wavy bond represents the connection to the payload. For (F3), (F4), (F8) and (F9), the payload can be connected to any one of the wavy bonds. The other wavy bond may then be connected to an R group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which (except hydrogen) may optionally be substituted and optionally interrupted by one or more heteroatoms selected from O, S and NR³² wherein R³² is independently selected from the group consisting of hydrogen and C₁-C₄ alkyl groups. The skilled person understands which R groups may be applied for each of the groups F. For example, the R group connected to the nitrogen atom of (F3) may be selected from alkyl and aryl, and the R group connected to the carbon atom of (F3) may be selected from hydrogen, alkyl, aryl, acyl and sulfonyl. Preferably, F² is selected from azides or tetrazines. Most preferably, F² is an azide.

### Application

The conjugates according to structure (1b) are especially suitable in the treatment of cancer. By virtue of the lack of an N-glycan, the conjugates according to structure (1b) will no longer be able to bind to Fc-gamma receptors, and therefore are highly effective in the treatment of cancer. The invention thus further concerns the use of the conjugate according to structure (1b) in medicine, preferably in the treatment of cancer. In a further aspect, the invention also concerns a method of treating a subject in need thereof, comprising administering the conjugate according to structure (1b) to the subject. The method according to this aspect can also be worded as the conjugate according to structure (1b) for use in treatment, in particular for use in the treatment of a subject in need thereof. The method according to this aspect can also be worded as use of the conjugate according to structure (1b) for the manufacture of a medicament. Herein, administration typically occurs with a therapeutically effective amount of the conjugate according to structure (1b).

The invention further concerns a method for the treatment of a specific disease in a subject in need thereof, comprising the administration of the conjugate according to the invention as defined above. Typically, the specific disease is cancer and the subject in need thereof is a cancer patient. The use of antibody-drug conjugates is well-known in cancer treatment, and the conjugates according to structure (1b) are especially suited in this respect. In the method according to this aspect, the conjugate is typically administered in a therapeutically effective amount. The present aspect of the invention can also be worded as a conjugate according to structure (1b) for use in the treatment of a specific disease in a subject in need thereof, preferably for the treatment of cancer. In other words, this aspect concerns the use of a conjugate according to structure (1b) for the preparation of a medicament or pharmaceutical composition for use in the treatment of a specific disease in a subject in need thereof, preferably for use in the treatment of cancer.

Administration in the context of the present invention refers to systemic administration. Hence, in one embodiment, the methods defined herein are for systemic administration of the conjugate. In view of the specificity of the conjugates, they can be systemically administered, and yet exert their activity in or near the tissue of interest (e.g. a tumour). Systemic administration has a great advantage over local administration, as the drug may also reach tumour metastasis not detectable with imaging techniques and it may be applicable to hematological tumours.

The invention further concerns a pharmaceutical composition comprising the conjugate according to structure (1b) and a pharmaceutically acceptable carrier.

### EXAMPLES

The invention is illustrated by the following examples.

### General Reagents and Analytics

Solvents were purchased from Sigma-Aldrich or Fisher Scientific and used as received. Thin layer chromatography was performed on silica gel-coated plates (Kieselgel 60 F254, Merck, Germany) with the indicated solvent mixture, spots were detected by KMnO₄ staining (1.5 g KMnO₄, 10 g K₂CO₃, 2.5 mL 5% NaOH-solution, 150 mL H₂O), p-anisaldehyde staining (9.2 mL p-anisaldehyde, 321 mL EtOH, 17 mL H₂O, 3.75 mL AcOH, 12.7 mL H₂SO₄), and UV-detection. NMR spectra were recorded on a Bruker Biospin 400 (400 MHz) and a Bruker DMX300 (300 MHz). Protein mass spectra (HRMS) were recorded on a JEOL AccuTOF JMS-T100CS (Electrospray Ionization (ESI) time-of-flight) or a JEOL AccuTOF JMS-100GCv (Electron Ionization (EI), Chemical Ionization (CI)). Low-resolution mass spectra (LRMS) were recorded on a ThermoScientific Advantage LCQ Linear ion-trap electrospray and a Waters LCMS consisting of a 2767 Sample manager, 2525 pump, 2996 UV-detector and a Micromass ZQ with an Xbridge™ C18 3.5 μm column (ESI).

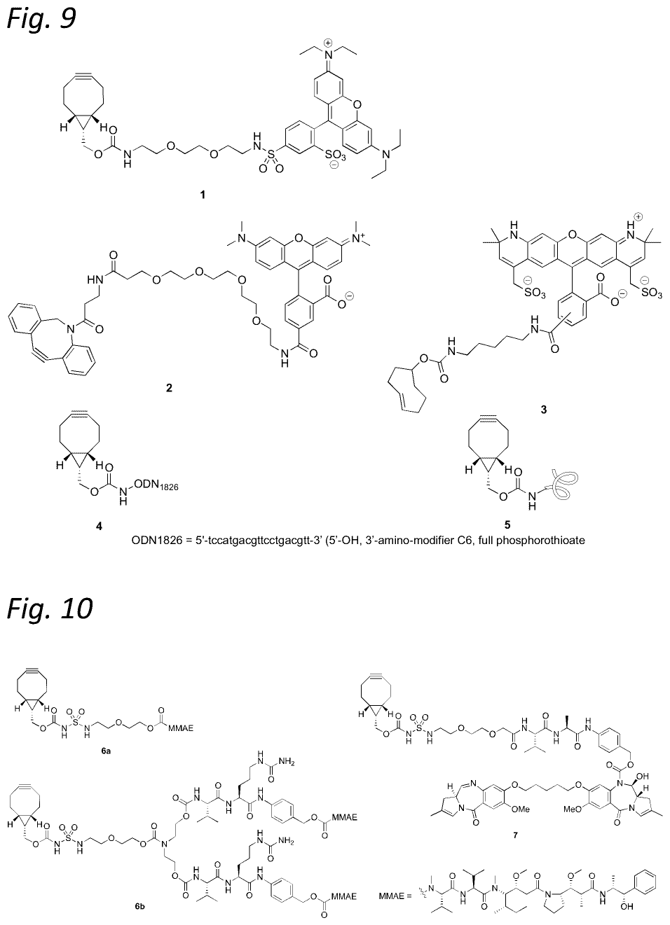

Trastuzumab (Herzuma) and cetuximab (Cerbitux) were obtained from the pharmacy. PNGase F was obtained from New England Biolabs (NEB). Compound 2 (structure in FIG. 9) was obtained from ClickChemistryTools (https://clickchemistrytools.com/product/tamra-dbco/). Compound 3 (structure in FIG. 9) was obtained from ClickChemistryTools (https://clickchemistrytools.com/product/af-568-tco). Compounds 4 (structure in FIG. 9) and 9d were custom synthesized by Eurogentec (www.eurogentec.com). Compound 9a was obtained from Broadpharm: https://broadpharm.com/web/product.php?catalog=BP-22443. Compounds 9b and 9c were prepared according to Bruins et al., ACS Omega 2019, 4, 11801-11807, incorporated by reference. Human IgG2 was purchased from Abcam (https://www.abcam.com/native-human-igg2-protein-ab90284.html?productWallTab=ShowAll#top-200). Mouse IgG1 was purchased from Abcam (https://www.abcam.com/mouse-igg1-kappa-monoclonal-mopc-21-isotype-control-ab18443.html).

### General Procedure for Reducing SDS-PAGE, Coomassie Staining and Fluorescence Detection

12% acrylamide gels were prepared according to BIO-RAD bulletin 6201 protocol. 5 μL 1 mg/mL antibody solution was diluted with 5 μL 2× sample buffer including 5% 2-mercaptoethanol and heated to 95° C. for 5 minutes. After loading the samples, the gel was run using a BIO-RAD Mini-PROTEAN Tetra Vertical Electrophoresis Cell at 150 volts until completion.

Fluorescently labelled proteins were analysed prior to staining using a BioRad ChemiDoc™ system. Subsequently, the gel was stained using staining solution, containing 1 g/L Coomassie Brilliant Blue R-250 in 5:4:1 (v/v/v) methanol:water:acetic acid, for 30 minutes. The gel was subsequently destained using 5:4:1 (v/v/v) methanol:water:acetic acid for 60 minutes, after which it was further destained overnight using demineralized water.

### General Procedure for Generation of Fc/2 Fragments

A solution of 20 μg of (modified) IgG was incubated for 1 hour at 37° C. with IdeS/Fabricator™ (1.25 UI/μL) in PBS pH 6.6 in a total volume of 10 μL.

### General Procedure for Analytical RP-HPLC

Prior to RP-HPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-HPLC analysis was performed on an Agilent 1100 series (Hewlett Packard). The sample (10 μL) was injected with 0.5 mL/min onto Bioresolve RP mAb 2.1*150 mm 2.7 μm (Waters) with a column temperature of 70° C. A linear gradient was applied in 16.8 minutes from 30 to 54% acetonitrile in 0.1% TFA and water.

### General Procedure for Analytical SEC

HPLC-SEC analysis was performed on an Agilent 1100 series (Hewlett Packard) using an Xbridge BEH200A (3.5 μM, 7.8×300 mm, PN 186007640 Waters) column. The sample was diluted to 1 mg/mL in PBS and measured with 0.86 mL/min isocratic method (0.1 M sodium phosphate buffer pH 6.9 (NaHPO₄/Na₂PO₄) containing 10% isopropanol) for 16 minutes.

### General Procedure for Analytical MS Analysis

Prior to mass spectral analysis, IgG was treated with IdeS, which allows analysis of the Fc/2 fragment. For analysis of the Fc/2 fragment, a solution of 20 μg (modified) IgG was incubated for 1 hour at 37° C. with IdeS/Fabricator™ (1.25 U/μL) in PBS pH 6.6 in a total volume of 10 μL. Samples were diluted to 80 μL followed by analysis electrospray ionization time-of-flight (ESI-TOF) on a JEOL AccuTOF. Deconvoluted spectra were obtained using Magtran software.

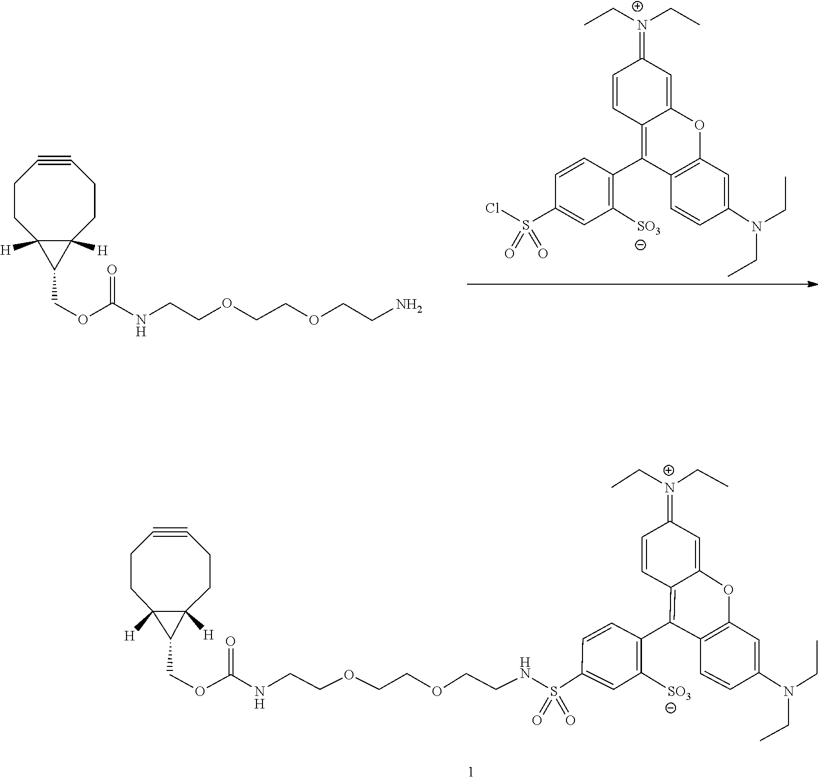

### Example 1. Synthesis of BCN-Lissamine Compound 1

Compound 1 was prepared by sulfonylation of BCN-diethyleneglycol-NH₂ (prepared as described for compound 24 in WO2014065661, example 1) with commercially available sulforhodamine B acid chloride (https://www.sigmaaldrich.com/catalog/product/sigma/86186).

### Example 2. Preparation of BCN-scFv Conjugate 5

BCN-UCHT1 conjugate was prepared according to Bartels et al., Methods 2019, 154, 93-101, incorporated by reference. Thus, 1 eq. of UCHT1-G₄SLPETGGH₆ (see sequence below) was incubated with 1 eq. sortase A and 30 eq of Gly₃-BCN tag (Figure). Typical conditions: To 100 μL 1.86 mg/mL UCHT1-G₄SLPETGGH₆ in TBS pH 8.0 was added 10 μL 17 mg/mL sortase A in TBS pH 8.0 (1 eq.), 13.6 μL 100 mM CaCl₂) in TBS pH 8.0, Gly₃-BCN in DMSO (4 μL 50 mM, 30 eq.), and 9.6 μL DMSO (10% final concentration), incubation overnight at 37° C. Unreacted UCHT1-G₄SLPETGGH₆ was removed by Ni-NTA column, and subsequent SEC-column yielded pure conjugate.

UCHT1 sequence:
VQLQQSGPELVKPGASMKISCKASGYSFTGYTMNWVKQSHGKNLEWMGL
INPYKGVSTYNQKFKDKATLTVDKSSSTAYMELLSLTSEDSAVYYCARS
GYYGDSDWYFDVWGAGTTVTVSSGGGSGGGSGGGSGGGSDIQMTQTTSS
LSASLGDRVTISCRASQDIRNYLNWYQQKPDGTVKLLIYYTSRLHSGVP
SKFSGSGSGTDYSLTISNLEQEDIATYFCQQGNTLPWTFAGGTKLEIKR
AGGGGSLPETGGHHHHHH

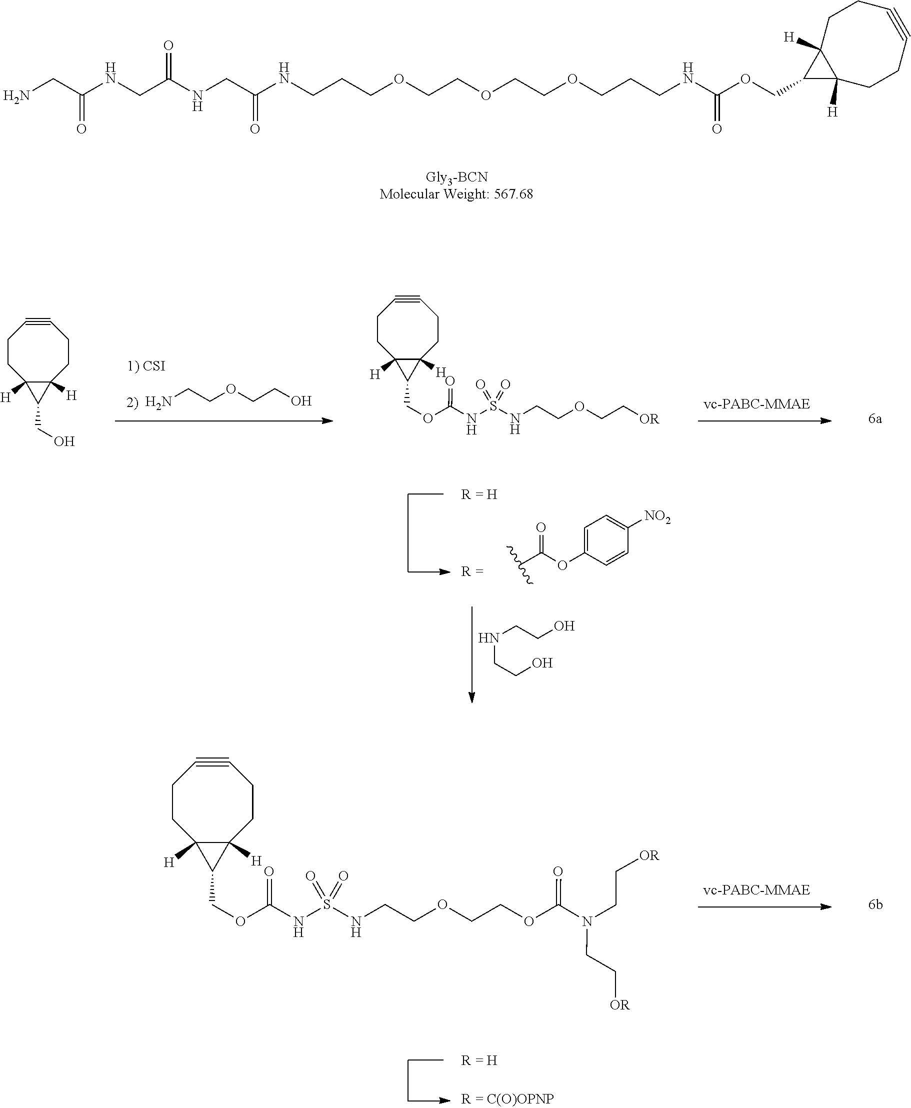

Chemical structures of 6a, 6b and 7 are depicted in FIG. 10.

### Example 3a. Synthesis of BCN-MMAE Compound 6a

Compound 6a (prepared according to procedure described by Verkade et al., Antibodies 2018, 7, doi:10.3390/antib7010012, incorporated by reference). To a solution of BCN alcohol (1.5 g, 10 mmol) in DCM (150 mL), under a N₂ atmosphere, was added CSI (0.87 mL, 1.4 g, 10 mmol), Et₃N (2.8 mL, 2.0 g, 20 mmol) and 2-(2-aminoethoxy)ethanol (1.2 mL, 1.26 g, 12 mmol). The mixture was stirred for 10 min and quenched through addition of aqueous NH₄Cl (sat., 150 mL). After separation, the aqueous layers was extracted with DCM (150 mL). The combined organic layers were dried (Na₂SO₄) and concentrated. The residue was purified with column chromatography. The product alcohol was obtained as slightly yellow thick oil (2.06 g, 5.72 mmol, 57%). ¹H NMR (400 MHz, CDCl₃) δ (ppm) 6.0 (bs, 1H), 4.28 (d, J=8.2 Hz, 2H), 3.78-3.73 (m, 2H), 3.66-3.61 (m, 2H), 3.61-3.55 (m, 2H), 3.34 (t, J=4.9 Hz, 2H), 2.37-2.15 (m, 6H), 1.64-1.48 (m, 2H), 1.40 (quintet, J=8.7 Hz, 1H), 1.05-0.92 (m, 2H).

To a solution of the alcohol prepared above (229 mg, 0.64 mmol) in DCM (20 mL) were added p-nitrophenyl chloroformate (128 mg, 0.64 mmol) and Et₃N (268 μL, 194 mg, 1.92 mmol). The mixture was stirred overnight at it and subsequently concentrated under reduced pressure. The residue was purified via gradient column chromatography (20→70% EtOAc in heptane (1% AcOH) to afford the PNP carbonate derivative as a white solid (206 mg, 0.39 mmol, 61%). ¹H NMR (400 MHz, CDCl₃) δ (ppm) 8.31-8.26 (m, 2H), 7.45-7.40 (m, 2H), 5.56 (t, J=6.0 Hz, 1H), 4.48-4.40 (m, 2H), 4.27 (d, J=8.2 Hz, 2H), 3.81-3.75 (m, 2H), 3.68 (t, J=5.0 Hz, 2H), 3.38-3.30 (m, 2H), 2.36-2.14 (m, 6H), 1.61-1.45 (m, 2H), 1.38 (quintet, J=8.7 Hz, 1H), 1.04-0.94 (m, 2H).

To a solution of the PNP carbonate prepared above (4.7 mg, 9.0 μmol) in DMF (200 μL) was added solid H-Val-Cit-PABC-MMAE (vc-PABC-MMAE, 10 mg, 8.1 μmol) followed by addition of Et₃N (3.7 μL, 2.7 mg, 27 μmol). After 23 h, 2′-(ethylenedioxy)bis(ethylamine) (1.3 μL, 1.3 mg, 8.9 μmol) in DMF was added (13 μL of 10% solution in DMF). The mixture was left for 4 h and purified via reversed phase (C18) HPLC chromatography (30→90% MeCN (1% AcOH) in H₂O (1% AcOH). The product 6a was obtained as a colourless film (10.7 mg, 7.1 μmol, 87%) LCMS (ESI+) calculated for C₇₄H₁₁₇N₁₂O₁₉S⁺ (M+H⁺) 1509.83 found 1510.59.

### Example 3b. Synthesis of BCN-MMAE Compound 6b

Compound 6b (prepared according to procedure for compound 7 described by Verkade et al., Antibodies 2018, 7, doi:10.3390/antib7010012, incorporated by reference). To solution of the PNP carbonate prepared in the synthesis of 6a (0.39 g; 0.734 mmol) in DCM (30 mL) were added a solution of diethanolamine (DEA, 107 mg; 1.02 mmol) in DMF (2 mL) and Et₃N (305 μL; 221 mg; 2.19 mmol). The resulting mixture was stirred at it for 17 h and washed with a saturated aqueous solution of NH₄Cl (30 mL). The aqueous phase was extracted with DCM (30 mL) and the combined organic layers were dried (Na₂SO₄) and concentrated. The residue was purified by flash column chromatography (DCM→MeOH/DCM 1/9). The product diol was obtained as a colourless film (163 mg; 0.33 mmol; 45%). ¹H NMR (400 MHz, CDCl₃) δ (ppm) 6.29 (bs, 1H), 4.33-4.29 (m, 2H), 4.28 (d, J=8.2 Hz, 2H), 3.90-3.80 (m, 4H), 3.69-3.64 (m, 2H), 3.61 (t, J=4.8 Hz, 2H), 3.52 (t, J=5.0 Hz, 4H), 3.32 (t, J=5.1 Hz, 2H), 2.37-2.18 (m, 6H), 1.60-1.55 (m, 2H), 1.39 (quintet, J=8.7 Hz, 1H), 1.05-0.94 (m, 2H).

To a solution of the diol prepared above (163 mg, 0.33 mmol) and 4-nitrophenyl chloroformate (134 mg, 0.66 mmol) in DCM (10 mL) was added Et₃N (230 μL; 167 mg; 1.65 mmol). The reaction mixture was stirred for 17 h and concentrated. The residue was purified by flash column chromatography (50% EtOAc in heptane→100% EtOAc). The product was obtained as a colourless oil (69 mg; 0.084 mmol; 25%). ¹H NMR (400 MHz, CDCl₃) δ (ppm) 8.29-8.23 (m, 4H), 7.42-7.35 (m, 4H), 5.81-5.71 (m, 1H), 4.53-4.43 (m, 4H), 4.36-4.30 (m, 2H), 4.25 (d, J=8.2 Hz, 2H), 3.81-3.70 (m, 4H), 3.70-3.65 (m, 2H), 3.62-3.56 (m, 2H), 3.32-3.24 (m, 2H), 2.34-2.14 (m, 6H), 1.60-1.45 (m, 2H), 1.35 (quintet, J=8.7 Hz, 1H), 1.02-0.91 (m, 2H).

To a solution of bis PNP-carbonate (27 mg, 33 μmol) in DMF (400 μL) were added triethylamine (22 μl; 16 mg; 158 μmol) and a solution of vc-PABC-MMAE.TFA (96 mg; 78 μmol) in DMF (1.0 mL). The mixture was left standing for 19 h and 2,2′-(ethylenedioxy)bis(ethylamine) (37 μL, 38 mg, 253 μmol) was added. After 2 h, the reaction mixture was diluted with DMF (100 μL) and purified by RP HPLC (C18, 30%→90% MeCN (1% AcOH) in water (1% AcOH). The desired product 6b was obtained as a colourless film (41 mg, 14.7 μmol, 45%). LCMS (ESI+) calculated for C₁₃₈H₂₁₉N₂₃O₃₅S²⁺ (M+2H⁺) 1395.79 found 1396.31.

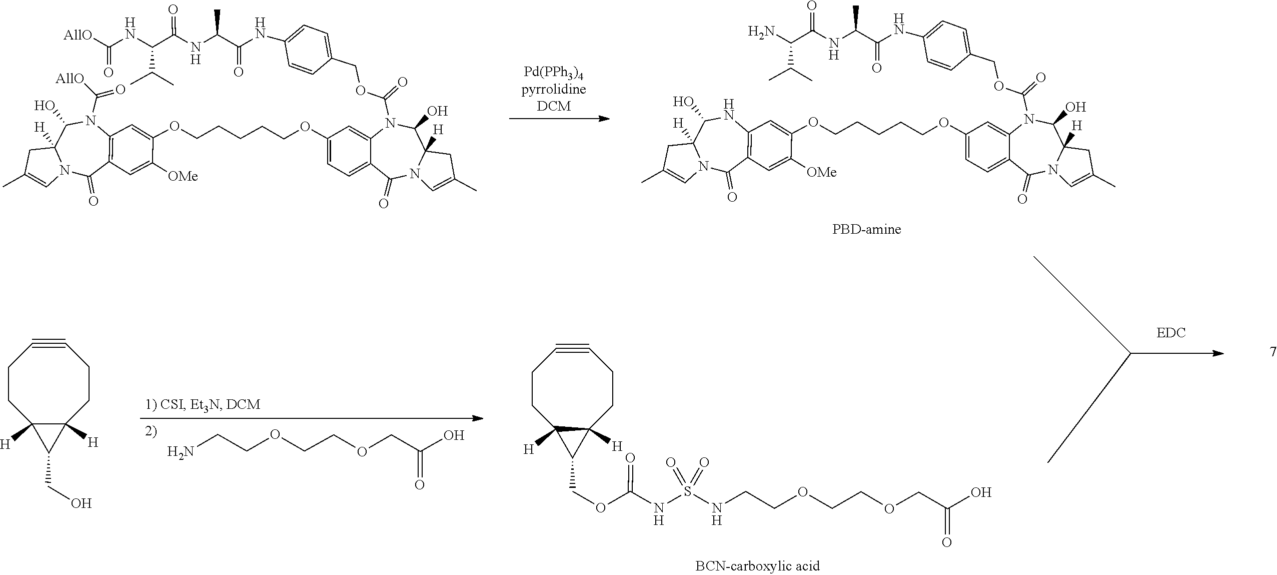

### Example 4. Preparation of BCN-PBD Compound 7

Compound 7 (prepared according to procedure described for compound 130 in WO2017137456, incorporated by reference).

### Preparation of BCN-Carboxylic Acid

A solution of BCN alcohol (0.384 g, 2.55 mmole) in MeCN (25 mL) under a N₂ atmosphere was cooled to 0° C., and chlorosulfonyl isocyanate was added (CSI) was added dropwise (0.255 mL, 415 mg, 2.93 mmole, 1.15 equiv.). After stirring for 15 minutes, Et₃N was added dropwise (1.42 mL, 1.03 g, 10.2 mmole, 4 equiv.) and stirring was continued for another 10 minutes. Next, a solution of 2-(2-(2-aminoethoxy)ethoxy)acetic acid (1.0 g, 6.1 mmole, 2.4 equiv.) in H₂O (5 mL) was added and the reaction mixture was stirred to room temperature for 2 h. After this time, CHCl₃ (50 mL) and H₂O (100 mL) were added, and the layers were separated. To the aqueous layer in a separatory funnel was added CH₂Cl₂ (100 mL) and the pH was adjusted to 4 with 1 N HCl, before separation of layers. The water layer was extracted twice with CH₂Cl₂ (2×100 mL), the organic layers were combined and dried (Na₂SO₄), filtered and concentrated. The residue was purified by flask column chromatography on silica, elution with CH₂Cl₂ to 20% MeOH in CH₂Cl₂. Yield 0.42 g (1.0 mmole, 39%) of BCN-carboxylic acid as a colorless sticky wax.

### Preparation of PBD-amine

Palladium tetrakistriphenylphosphine Pd(PPh₃)₄ (4.8 mg, 4.15 μmol) is weighed and put under an atmosphere of N₂. A solution of pyrrolidine (5.0 μL, 4.3 mg, 60 μmol) in DCM (1 mL) is degassed by bubbling N₂ through the solution. A solution of Alloc-protected PBD amine (27 mg, 24 μmol) in DCM (6 mL) is degassed by bubbling N₂ through the solution. While N₂ is still bubbled through the solution, the degassed solution of pyrrolidine is added. The weighed Pd(PPh₃)₄ is dissolved in CH₂Cl₂ (1 mL) and 0.9 mL of this solution is added. After 50 min of bubbling of N₂, CH₂Cl₂ (25 mL) is added and the mixture is washed with aqueous saturated NH₄Cl (25 mL). After separation, the aqueous layer is extracted with CH₂Cl₂ (2×25 mL). The combined organic layers are dried (Na₂SO₄) and concentrated. The residue is purified by RP-HPLC (30-90% MeCN (0.1% formic acid) in H₂O (0.1% formic acid). The combined fractions are passed through SPE (HCO₃⁻) columns and concentrated. After addition of MeCN (50 mL) the mixture is again concentrated. The resulting residue is used in the next step.

### Preparation of BCN-PBD Compound 7

To a solution of PBD-amine in CHCl₃ (5 mL) is added a solution of BCN-carboxylic acid (15 mg, 36 μmol) in CHCl₃ (0.8 mL). The resulting mixture is added to solid EDC·HCl (4.7 mg, 25 μmol), CHCl₃ (5 mL) was added and the mixture was left standing for 16 h. DCM (30 mL) is added and the resulting mixture is washed with water (30 mL). After separation, the aqueous phase is extracted with DCM (30 mL). The combined organic layers are dried (Na₂SO₄) and concentrated. The residue is purified by RP-HPLC (30-90% MeCN (no acid) in H₂O (0.01% formic acid). The HPLC collection tubes are filled with 5% aqueous (NH₄)HCO₃ before collection. The combined HPLC fractions are extracted with DCM (3×20 mL). The combined organic layers are dried (Na₂SO₄) and concentrated. The product 7 is obtained as slightly yellow oil (21 mg, 16 μmol, mw 1323 g/mole, 67% over two steps from Alloc-protected PDB amine).

### Example 5. Synthesis of TCO-OSu

The starting TCO-OH (prepared as described by Blackman et al., J. Am. Chem. Soc. 2008, 130, 41, 13518-13519, incorporated by reference) (120 mg, 0.953 mmol, 1 eq.) was dissolved in 5 mL dry DCM under nitrogen. Triethylamine (193 mg, 1.91 mmol, 2 eq.) and N,N′-disuccinimidyl carbonate (269 mg, 1.05 mmol, 1.1 eq.) were added and stirred until TLC indicated completion (16 h at rt). The sample was concentrated under vacuo and purified by flash column chromatography (20-30% EtOAc in n-heptane), yielding TCO-Osu (192 mg, 0.720 mmol, 76% yield).

### Example 6. Synthesis of Bifunctional BCN-TCO Compound 8

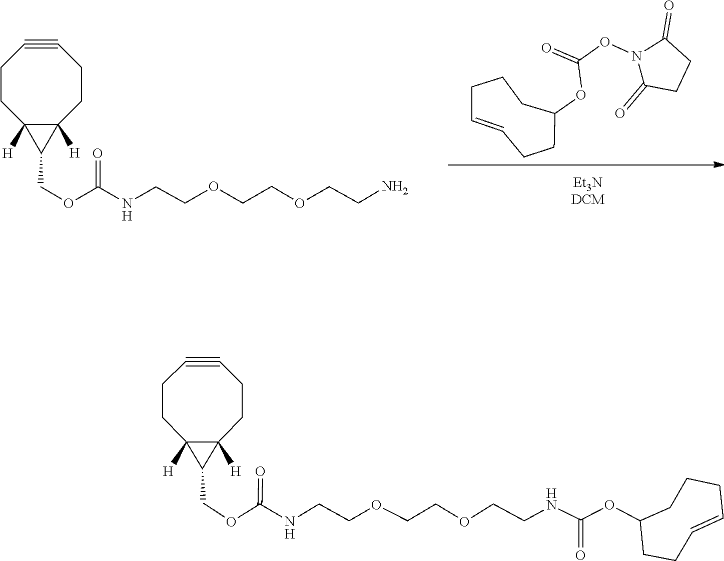 [SMILES: NCCOCCOCCNC(=O)OC[C@@H]1[C@@H]2CCC#CCC[C@@H]21 ; O=C(OC1CC/C=C/CCC1)ON1C(=O)CCC1=O ; O=C(NCCOCCOCCNC(=O)OC1CC/C=C/CCC1)OC[C@@H]1[C@@H]2CCC#CCC[C@@H]21]

BCN-diethyleneglycol-NH₂ (prepared as described in WO2014065661, example 1) (20.1 mg, 0.0620 mmol, 1 eq) was dissolved in 2 mL dry DCM under nitrogen. Triethylamine (12.5 mg, 0.124 mmol, 2 eq) was added. TCO-OSu (19.9 mg, 0.0743 mmol, 1.2 eq) was added. The reaction was stirred until TLC indicated completion (2 h at RT). The sample was concentrated under vacuo and purified by flash column chromatography (5% MeOH in DCM).

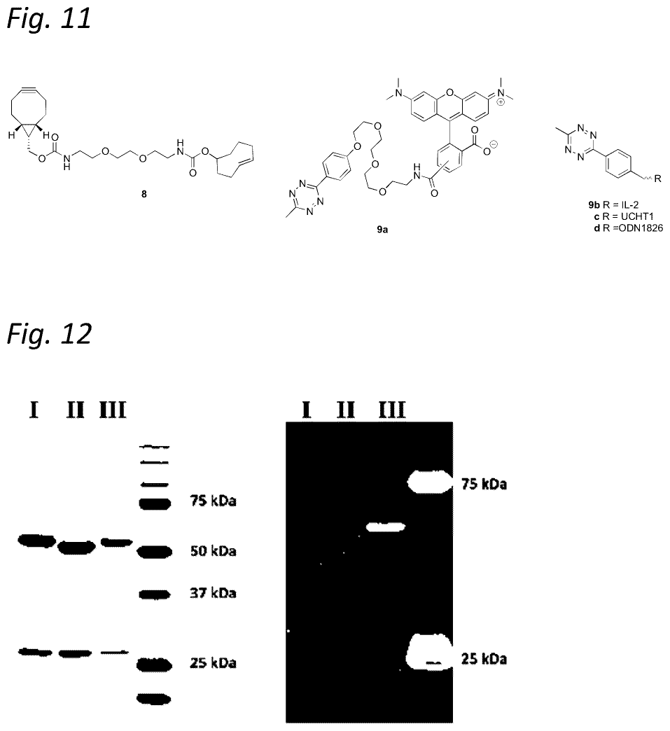

Chemical structures of 8, 9a-d are depicted in FIG. 11.

### Example 7. Preparation of Methyltetrazine-IL-2 Compound 9b

MeTz-IL-2 conjugate 9b was prepared according to Bartels et al., Methods 2019, 154, 93-101, incorporated by reference. Thus, 1 eq. of IL-2-G₄SG₄SLPETGGH₆ (see sequence below) was incubated with 1 eq. sortase A and 30 eq of Gly₃-MeTz tag (Figure). Typical conditions: To 100 μL 1.2 mg/mL IL-2-G₄SGG₄SLPETGGH₆ in TBS pH 8.0 was added 10 μL 17 mg/mL sortase A in TBS pH 8.0 (1 eq.), 13.6 μL 100 mM CaCl₂ in TBS pH 8.0, Gly₃-MeTz in DMSO (4 μL 50 mM, 30 eq.), and 9.6 μL DMSO (10% final concentration), incubate overnight at 37° C. overnight. Unreacted IL-2-G₄SGG₄SLPETGGH₆ was removed by Ni-NTA column, and subsequent SEC-column yielded pure conjugate.

IL-2 sequence:
APTSSSTKKTQLQLEHLLLDLQMILNGINNYKNPKLTAMLTKKFYMPKK
ATELKHLQCLEEELKPLEEVLNLAQSKNFHLRPRDLISNINVIVLELKG
SETTFMCEYADETATIVEFLNRWITFSQSIISTLT
IL-2-G₄SGG₄SLPETGGH₆ sequence:
APTSSSTKKTQLQLEHLLLDLQMILNGINNYKNPKLTAMLTKKFYMPKK
ATELKHLQCLEEELKPLEEVLNLAQSKNFHLRPRDLISNINVIVLELKG
SETTFMCEYADETATIVEFLNRWITFSQSIISTLTGGGGSGGGGSLPET
GGHHHHHH

### Example 8. Preparation of methyltetrazine-UCHT1 Compound 9c

MeTz-UCHT1 conjugate was prepared according to Bartels et al., Methods 2019, 154, 93-101, incorporated by reference. Thus, 1 eq. of UCHT1-G₄SLPETGGH₆ (see sequence above) was incubated with 1 eq. sortase A and 30 eq of Gly₃-MeTz. Typical conditions: To 100 μL 2 mg/mL UCHT1-G₄SLPETGGH₆ in TBS pH 8.0 was added 10 μL 17 mg/mL sortase A in TBS pH 8.0 (1 eq.), 13.6 μL 100 mM CaCl₂ in TBS pH 8.0, Gly₃-MeTz in DMSO (4 μL 50 mM 30 eq.), and 9.6 μL DMSO (10% final concentration), incubation at 37° C. overnight. Unreacted UCHT1-G₄SLPETGGH₆ was removed by Ni-NTA column, and subsequent SEC-column yielded pure conjugate.

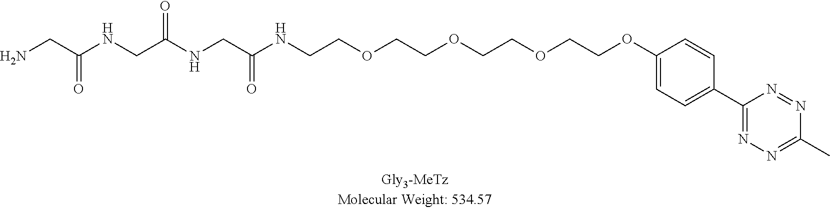 [SMILES: Cc1nnc(-c2ccc(OCCOCCOCCOCCNC(=O)CNC(=O)CNC(=O)CN)cc2)nn1]

### Example 9. Enzymatic deglycosylation of trastuzumab or cetuximab with PNGase F

Trastuzumab (Herzuma) (12 mg, 18.4 mg/mL in PBS pH 7.4) was incubated with PNGase F (15 μL, 7500 units) at 37° C. After overnight incubation the antibody was dialyzed (3 times to PBS pH 5.5) and concentrated to 15.3 mg/mL. Mass spectral analysis of a sample after IdeS treatment showed one major Fc/2 product (observed mass 23787 Da) corresponding to the expected product.

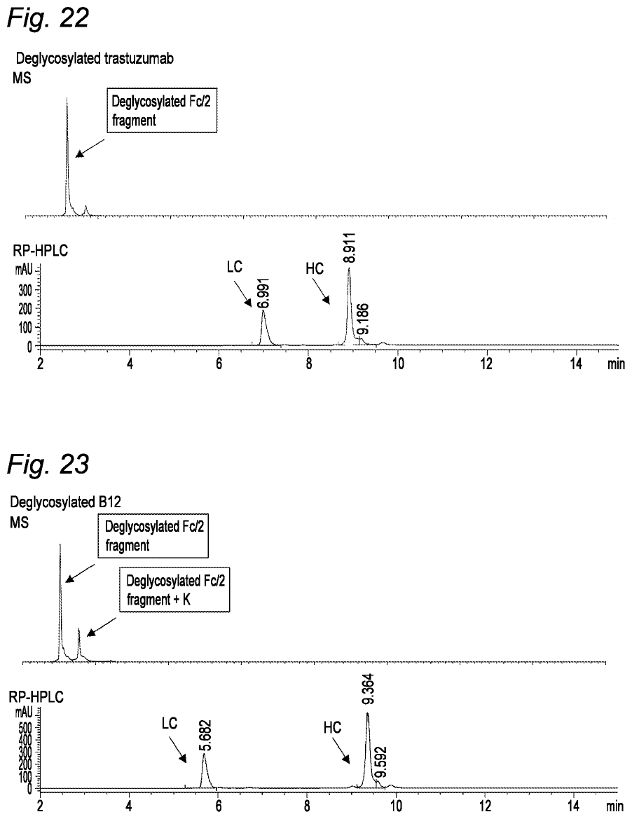

Cetuximab (Cerbitux) was deglycosylated similarly. Mass spectral analysis of a sample after IdeS treatment showed one major Fc/2 product (observed mass 23,787 Da) corresponding to the expected product. HPLC-profiles for deglycosylated trastuzumab and cetuximab are depicted in FIGS. 22 and 23, respectively.

### Example 10. Conjugation of deglycosylated trastuzumab with 1

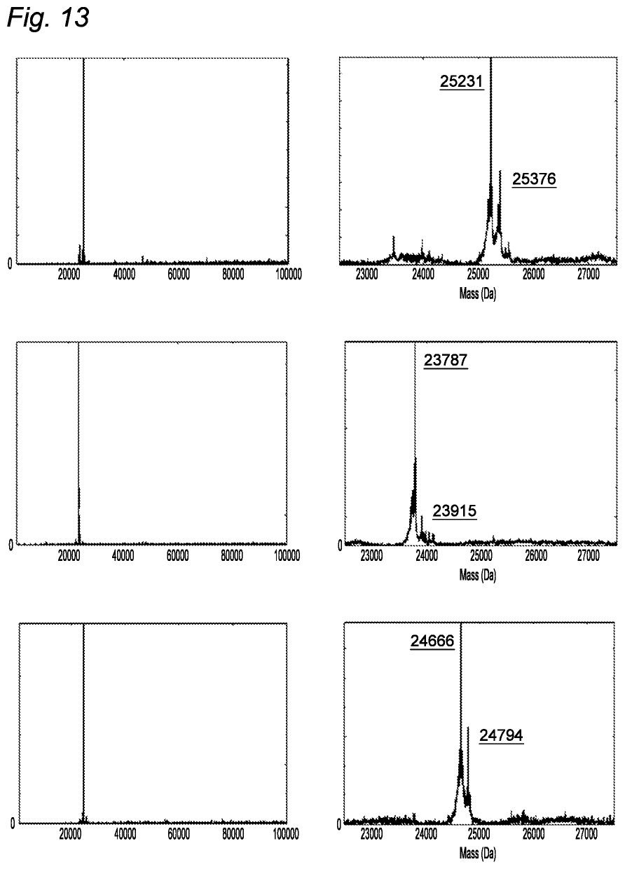

Deglycosylated trastuzumab (8.0 μL, 5.0 mg/mL, 40 μg in PBS pH 5.5) was diluted with 4.8 μL PBS pH 5.5 and incubated with 1 (1.6 μL, 2.0 mg/mL, 2.3 mM in DMF or DMSO) and mushroom tyrosinase (1.6 μL, 10 mg/mL in phosphate buffer pH 6.0) for 16 h at 4° C. After completion, the product was purified using protein A purification. SDS-PAGE was performed as described above, this showed the formation of a fluorescent band on the heavy chain of trastuzumab (FIG. 12). Mass spectral analysis of a sample after IdeS treatment showed one major Fc/2 product (observed mass 24666 Da) corresponding to the expected product (FIG. 13). HPLC analysis was performed as described above and indicated clean conversion (FIG. 17).

### Example 11. Conjugation of deglycosylated cetuximab with 1

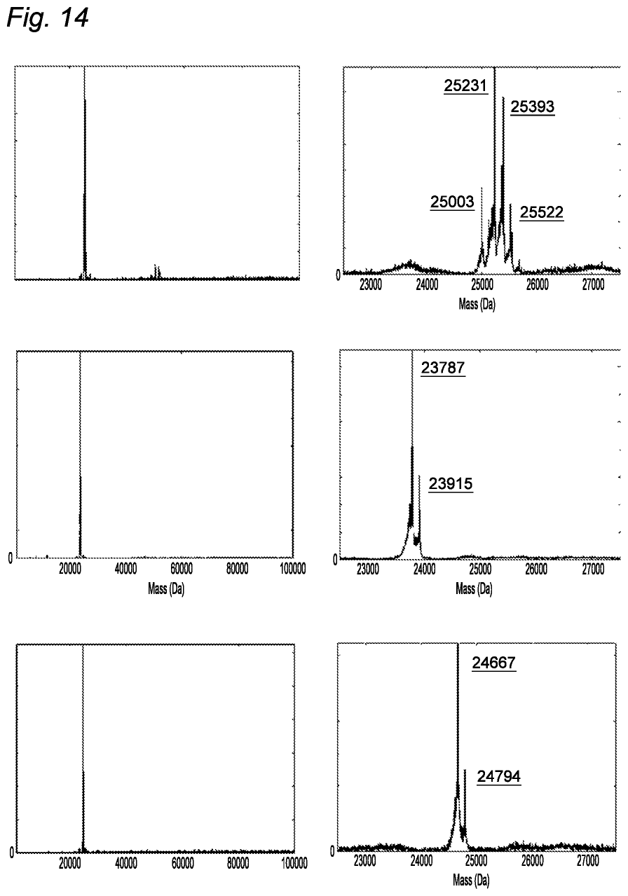

Deglycosylated cetuximab (8.0 μL, 5.0 mg/mL, 40 μg in PBS pH 5.5) was diluted with 4.8 μL PBS pH 5.5 and incubated with 1 (1.6 μL, 2.0 mg/mL, 2.3 mM in DMF or DMSO) and mushroom tyrosinase (1.6 μL, 10 mg/mL in phosphate buffer pH 6.0) for 16 h at 4° C. After completion, the product was purified using protein A purification. Mass spectral analysis of a sample after IdeS treatment showed one major Fc/2 product (observed mass 24667 Da) corresponding to the expected product (FIG. 14).

### Example 12. Evaluation of Stoichiometry of BCN-lissamine 1 for Labeling of cetuximab

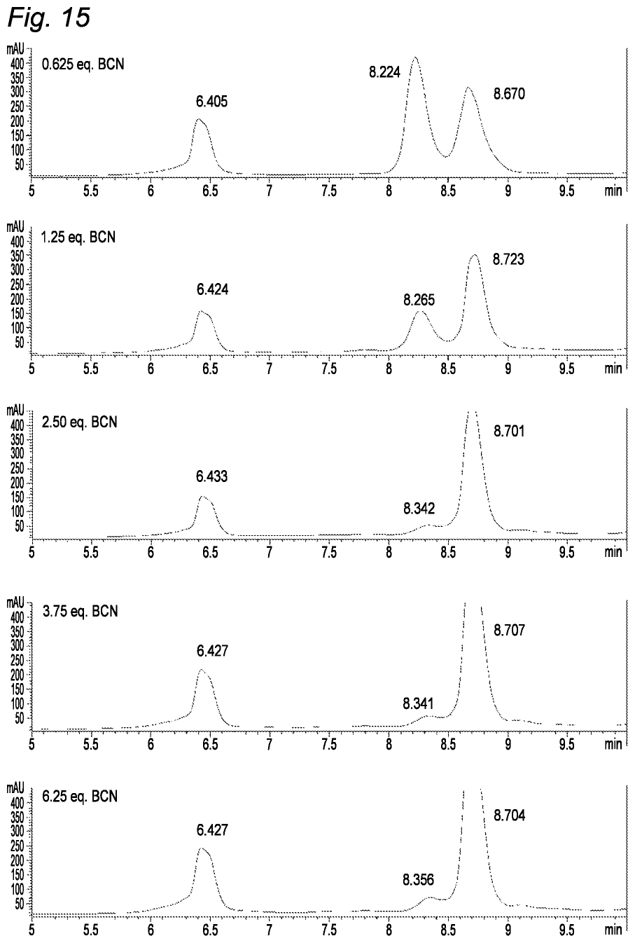

To evaluate the effect of stoichiometric ratio of 1 versus deglycosylated cetuximab, various concentrations of 1 were incubated with deglycosylated cetuximab in presence of mushroom tyrosinase. 1.39 μL 7.2 mg/mL deglycosylated cetuximab in PBS pH 5.5 was diluted with 3 uL PBS pH 5.5. To the mixture was added 0.5 μL 10 mg/mL mushroom tyrosinase in phosphate buffer pH 6.0 and 0.5 μL BCN-lissamine 1 in DMSO with varying concentrations for each sample (see table). The samples were reacted for 24 h at 4° C., after which conversion was determined by HPLC (FIG. 15, and table below).

| | Concentration 1 | 1 (eq. per | |
|---|---|---|---|
| Sample # | (mg/mL) | tyrosine) | Conversion (%) |
| 1 | 0.145 | 0.625 | 46 |
| 2 | 0.289 | 1.25 | 69 |
| 3 | 0.578 | 2.5 | 93 |
| 4 | 0.867 | 3.75 | 95 |
| 5 | 1.445 | 6.25 | 93 |

### Example 13. Evaluation of Labeling of trastuzumab and cetuximab with TCO-AF₅₆₈ (3)

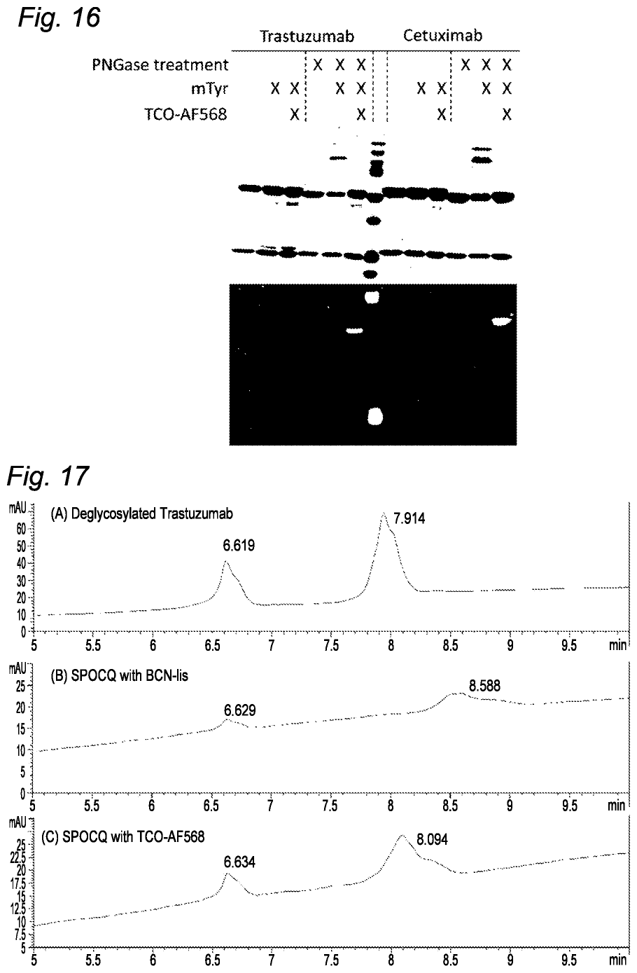

Deglycosylated cetuximab (8.0 μL, 5.0 mg/mL, 40 μg in PBS pH 5.5) or trastuzumab (similar amount) was diluted with 4.8 μL PBS pH 5.5 and incubated with 3 (1.6 μL, 4.0 mg/mL, 4.3 mM in DMF or DMSO) and mushroom tyrosinase (1.6 μL, 10 mg/mL in phosphate buffer pH 6.0) for 16 h at 4° C. After completion, the product was purified using protein A purification. SDS-PAGE was performed as described above, this showed the formation of a fluorescent band on the heavy chain of trastuzumab and cetuximab (FIG. 16). HPLC analysis was performed as describe above and indicated clean conversion (FIG. 17).

### Example 14: Attempted Labelling of Intact Mouse IqG1 with 3

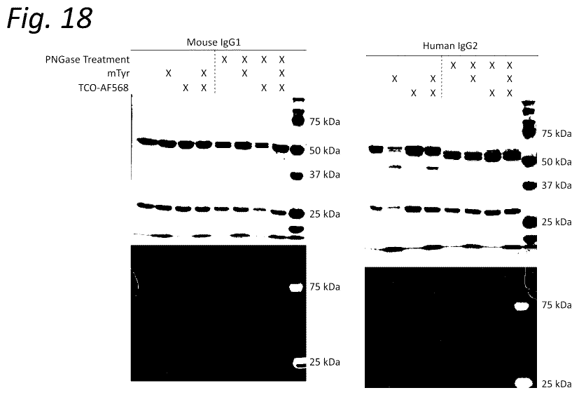

Mouse IgG1 in PBS pH 7.1 (10 μL, 0.5 mg/mL) was incubated with TCO-AF568 3 (1.0 μL, 4.0 mg/mL in DMSO, 65 eq.) and mushroom tyrosinase (1.0 μL, 10 mg/mL in phosphate buffer pH 6.5) for 48 h at 4° C. SDS-PAGE analysis was performed as described above (FIG. 18). No oxidation of the antibody was observed.

### Example 15: Attempted Labelling of deglycosylated Mouse IqG1 with 3

Mouse IgG1 in PBS pH 7.1 (200 μL, 0.5 mg/mL) was incubated with PNGase F (10 μL 0.1 mg/mL) for 16 hours at 37° C. The reaction was rebuffered to PBS pH 7.1 with spin-filtration (MWCO 100 kDa), which removed PNGase F. The deglycosylated mouse IgG1 (1.1 μL, 4.5 mg/mL) was diluted with 6.9 μL PBS pH 7.1, and incubated with TCO-AF568 3 (1.0 μL, 4.0 mg/mL in DMSO, 65 eq.) and mushroom tyrosinase (1.0 μL, 10 mg/mL in phosphate buffer pH 6.5) for 48 h at 4° C. SDS-PAGE analysis was performed as described above (FIG. 18). No oxidation of the antibody was observed.

### Example 16: Attempted Labelling of Intact Human IqG2 with 3

Human IgG2 in PBS pH 7.1 (2.5 μL, 2.1 mg/mL) was diluted with 5.5 μL PBS pH 7.1, and incubated with TCO-AF568 3 (1.0 μL, 4.0 mg/mL in DMSO, 62 eq.) and mushroom tyrosinase (1.0 μL, 10 mg/mL in phosphate buffer pH 6.5) for 48 h at 4° C. SDS-PAGE analysis was performed as described above (FIG. 18). No oxidation of the antibody was observed.

### Example 17: Attempted Labelling of deglycosylated Human IqG2 with 3

Human IgG2 in PBS pH 7.1 (50 μL, 2.1 mg/mL) was incubated with PNGase F (10 μL 0.1 mg/mL) for 16 hours at 37° C. The reaction was rebuffered to PBS pH 7.1 with spin-filtration (MWCO 100 kDa), which removed PNGase F. The deglycosylated human IgG2 (1 μL, 4.8 mg/mL) was diluted with 7.0 μL PBS pH 7.1, and incubated with TCO-AF568 3 (1.0 μL, 4.0 mg/mL in DMSO, 67 eq.) and mushroom tyrosinase (1.0 μL, 10 mg/mL in phosphate buffer pH 6.5) for 48 h at 4° C. SDS-PAGE analysis was performed as described above (FIG. 18). No oxidation of the antibody was observed.

### Example 19. Competition Experiment for Labelling of trastuzumab LC-G₄Y Between BCN-Reagent 1 and TCO-Reagent 3

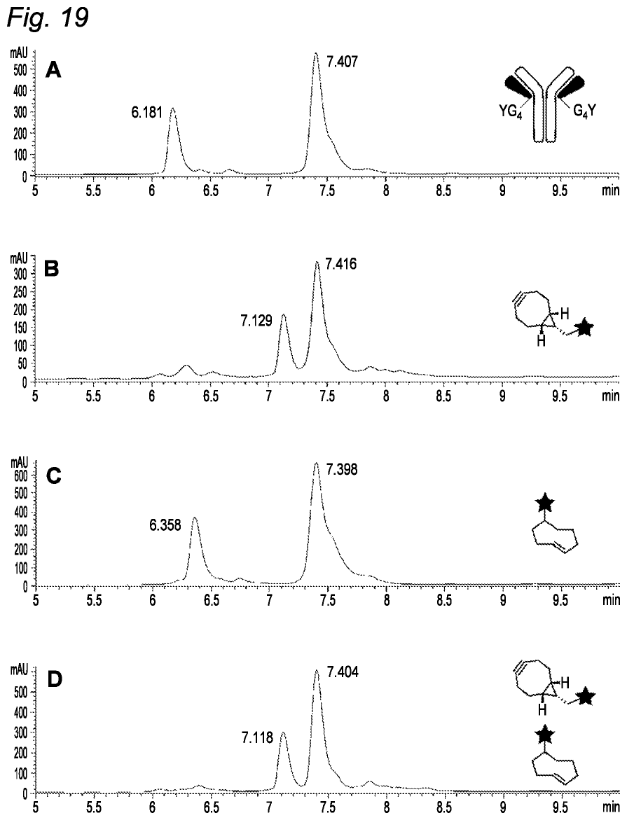

(A) Tras[LC]G₄SG₄SG₄Y in PBS pH 5.5 (1.73 μL, 28.9 mg/mL, 50 μg) was diluted with 8.27 μL PBS and incubated 16 hours at 4° C. HPLC analysis was performed as described above, and showed a clean light and heavy chain trace for Tras[LC]G₄SG₄SG₄Y. The HPLC-trace is depicted in FIG. 19A.
(B) Tras[LC]G₄SG₄SG₄Y in PBS pH 5.5 (1.73 μL, 28.9 mg/mL, 50 μg) was diluted with 6.78 μL PBS pH 5.5. To the solution was added BCN-lissamine 1 (0.5 μL, 5.0 mg/mL, 2.5 μg, 4.3 eq. per tyrosine tag). After homogenizing the sample, mushroom tyrosinase (1.0 μL, 10 mg/mL in phosphate buffer pH 6.0) was added. The mixture was reacted for 16 h at 4° C. HPLC analysis was performed as described above, and showed a clean conversion on the light chain of Tras[LC]G₄SG₄SG₄Y with a 1 minute shift in retention time. The HPLC-trace is depicted in FIG. 19B.
(C) Tras[LC]G₄SG₄SG₄Y in PBS pH 5.5 (1.73 μL, 28.9 mg/mL, 50 μg) was diluted with 6.65 μL PBS pH 5.5. To which was added TCO-AF568 3 (0.625 μL 4.0 mg/mL, 2.5 μg, 4.03 eq. per tyrosine tag). After homogenizing the sample mushroom tyrosinase (1.0 μL, 10 mg/mL in phosphate buffer pH 6.0) was added. The mixture was reacted for 16 h at 4° C. HPLC analysis was performed as described above, and showed a clean conversion on the light chain of Tras[LC]G₄SG₄SG₄Y with a 0.2 minute shift in retention time. The HPLC-trace is depicted in FIG. 19C.
(D) Tras[LC]G₄SG₄SG₄Y in PBS pH 5.5 (1.73 μL, 28.9 mg/mL, 50 μg) was diluted with 6.15 μL PBS pH 5.5. To which was added BCN-lissamine 1 (0.5 μL, 5.0 mg/mL, 2.5 μg, 4.3 eq. per tyrosine tag) and TCO-AF568 3 (0.625 μL 4.0 mg/mL, 2.5 μg, 4.03 eq. per tyrosine tag). After homogenizing the sample mushroom tyrosinase (1.0 μL, 10 mg/mL in phosphate buffer pH 6.0) was added. HPLC analysis was performed as described above, and showed a clean conversion on the light chain of Tras[LC]G₄SG₄SG₄Y with a 1 minute shift in retention time, indicating the formation of primarily BCN-conjugate. The HPLC-trace is depicted in FIG. 19D.

Tras[LC]G₄SG₄SG₄Y sequence:
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECGGGGSGGGGSGGGGY

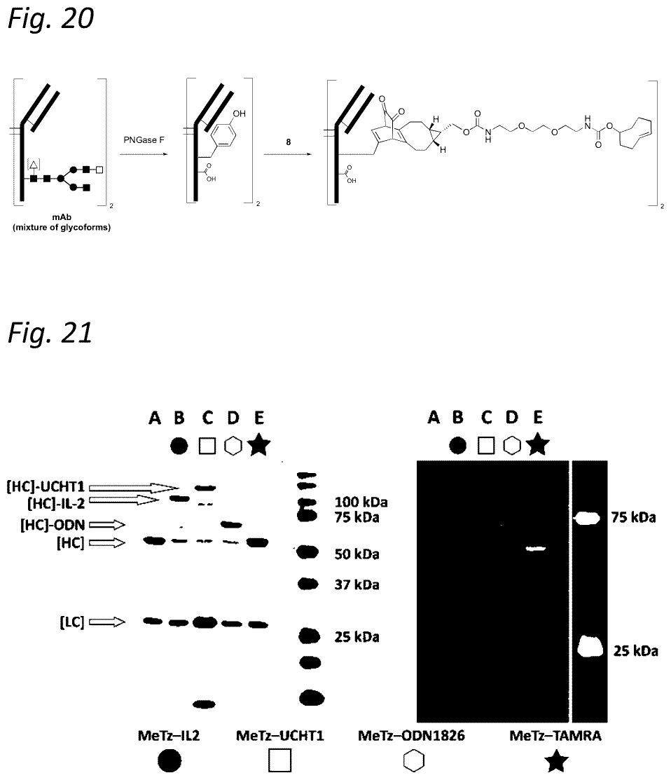

### Example 20. Conjugation of deglycosylated cetuximab with Bifunctional BCN-TCO Reagent 8 Leading to cetuximab-TCO (Conceptually Depicted in FIG. 20)

Deglycosylated cetuximab (11.0 μL, 9.0 mg/mL in PBS pH 5.5) was diluted with 33.0 μL PBS pH 5.5. To which was added BCN-TCO 8 (5.5 μL, 5.0 mg/mL, 27.5 μg, 44 eq. pertyrosine tag, in DMSO) and subsequently was added mushroom tyrosinase (5.5 μL, 10 mg/mL in phosphate buffer pH 6.0). The mixture was reacted for 16 h at 4° C. The reaction was rebuffered to PBS pH 7.1 with spin-filtration (MWCO 100 kDa), which removed unreacted BCN-TCO 8. TCO-modified cetuximab final concentration was 5.2 mg/mL.

### Example 21. Reaction of cetuximab-TCO with methyltetrazine Reagents 9a-9d

TCO-modified cetuximab (1.0 μL, 5.2 mg/mL) was diluted with 3.5 μL PBS pH 7.1 and subsequently incubated with MeTz-TAMRA 9a (0.5 μL, 1.0 mg/mL, 9.3 eq. perTCO) in DMSO. The sample was incubated at 4° C. for 30 minutes. SDS-PAGE analysis was performed as described above, this showed formation of a fluorescent band at the heavy chain (FIG. 21E).

TCO-modified cetuximab (1.0 μL, 5.2 mg/mL) was diluted with 3.5 μL PBS pH 7.1 and subsequently incubated with MeTz-IL2 9b (0.5 μL, 7.4 mg/mL, 3.0 eq. per TCO) in PBS pH 7.1. The sample was incubated at 4° C. for 30 minutes. SDS-PAGE analysis was performed as described above, this showed formation of a fluorescent band at the heavy chain (FIG. 21B).

TCO-modified cetuximab (1.0 μL, 5.2 mg/mL) was diluted with 3.33 μL PBS pH 7.1 and subsequently incubated with MeTz-UCHT1 9c (0.67 μL, 9.1 mg/mL, 3.1 eq. per TCO) in PBS pH 7.1. The sample was incubated at 4° C. for 30 minutes. SDS-PAGE analysis was performed as described above, this showed formation of a fluorescent band at the heavy chain (FIG. 21C).

TCO-modified cetuximab (1.0 μL, 5.2 mg/mL) was diluted with 2.0 μL PBS pH 7.1 and subsequently incubated with MeTz-ODN1826 9d (2.0 μL, 100 μM, 2.8 eq. per TCO) in MilliQ. The sample was incubated at 4° C. for 30 minutes. SDS-PAGE analysis was performed as described above, this showed formation of a fluorescent band at the heavy chain (FIG. 21D).

### Example 22. Transient Expression and Purification of B12

B12 was transiently expressed in CHO K1 cells by Evitria (Zurich, Switzerland) at 1 L scale. The supernatant was purified using a protein A column (25 mL, CaptivA PriMAB). The supernatant was loaded onto the column followed by washing with at least 10 column volumes of 25 mM Tris pH 7.5, 150 mM NaCl (TBS). Retained protein was eluted with 0.1 M NaOAc pH 3.5. The eluted product was immediately neutralized with 2.5 M Tris-HCl pH 7.2 and dialyzed against TBS. Next, the IgG was concentrated (15-20 mg/mL) using a Vivaspin Turbo 4 ultrafiltration unit (Sartorius).

### Example 23. Enzymatic deglycosylation of B12 with PNGase F

B12 (6 mg, 10 mg/mL in PBS pH 7.4) was incubated with PNGase F (6 μL, 3000 units, NEB) at 37° C. After overnight incubation the antibody was dialyzed (3 times to PBS pH 5.5) and concentrated to 23.6 mg/mL. Mass spectral analysis of a sample after IdeS treatment showed one major Fc/2 product (observed mass 23756 Da, approximately 70% of total Fc/2) corresponding to the expected product and a minor product (observed mass 23885 Da, approximately 25% of total Fc/2) for the expected product+lysine.

### Example 24. Conjugation of deglycosylated trastuzumab with BCN-HS-PEG₂-vc-PABC-MMAE (6a)

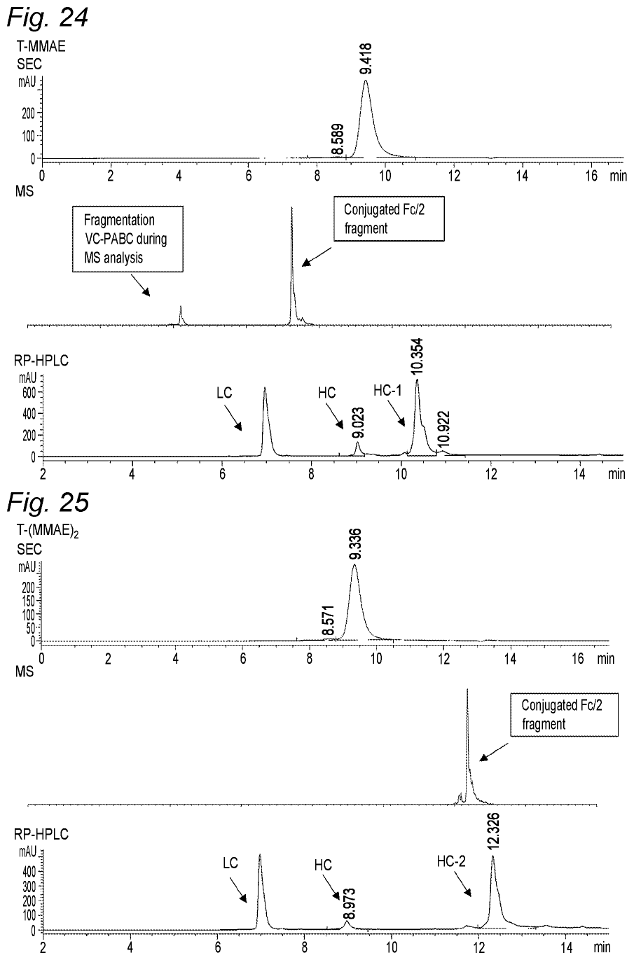

Deglycosylated trastuzumab (196 μL, 3 mg, 15.3 mg/mL in PBS 5.5) was incubated with BCN-HS-PEG₂-vc-PABC-MMAE 6a (40 μL, 5 mM in DMF) and mushroom tyrosinase (60 μL, 10 mg/mL in phosphate buffer pH 6.0, Sigma Aldrich T3824) for 16 h. Subsequently, the reaction was diluted with 300 μL PBS and centrifuged for 2 min at 13.000 rpm. The liquid was purified on a Superdex200 Increase 10/300 GL (GE Healthcare) column on an AKTA Purifier-10 (GE Healthcare). Mass spectral analysis of the IdeS-digested sample showed one major product (observed mass 25311 Da, approximately 90% of total Fc/2 fragment), corresponding to the conjugated Fc/2 fragment. SEC, MS and HPLC profiles of the conjugate depicted in FIG. 24.

### Example 25. Conjugation of deglycosylated trastuzumab with BCN-HS-PEG₂-(vc-PABC-MMAE)₂ (6b)

Deglycosylated trastuzumab (196 μL, 3 mg, 15.3 mg/mL in PBS 5.5) was incubated with BCN-HS-PEG₂-(vc-PABC-MMAE)₂ 6b (40 μL, 5 mM in DMF) and mushroom tyrosinase (60 μL, 10 mg/mL in phosphate buffer pH 6.0, Sigma Aldrich T3824) for 16 h. Subsequently, the reaction was diluted with 300 μL PBS and centrifuged for 2 min at 13.000 rpm. The liquid was purified on a Superdex200 Increase 10/300 GL (GE Healthcare) column on an AKTA Purifier-10 (GE Healthcare). Mass spectral analysis of the IdeS-digested sample showed one major product (observed mass 26591 Da, approximately 90% of total Fc/2 fragment), corresponding to the conjugated Fc/2 fragment. SEC, MS and HPLC profiles of the conjugate depicted in FIG. 25.

### Example 26. Conjugation of deglycosylated trastuzumab with BCN-HS-PEG₂-va-PABC-PBD (7)

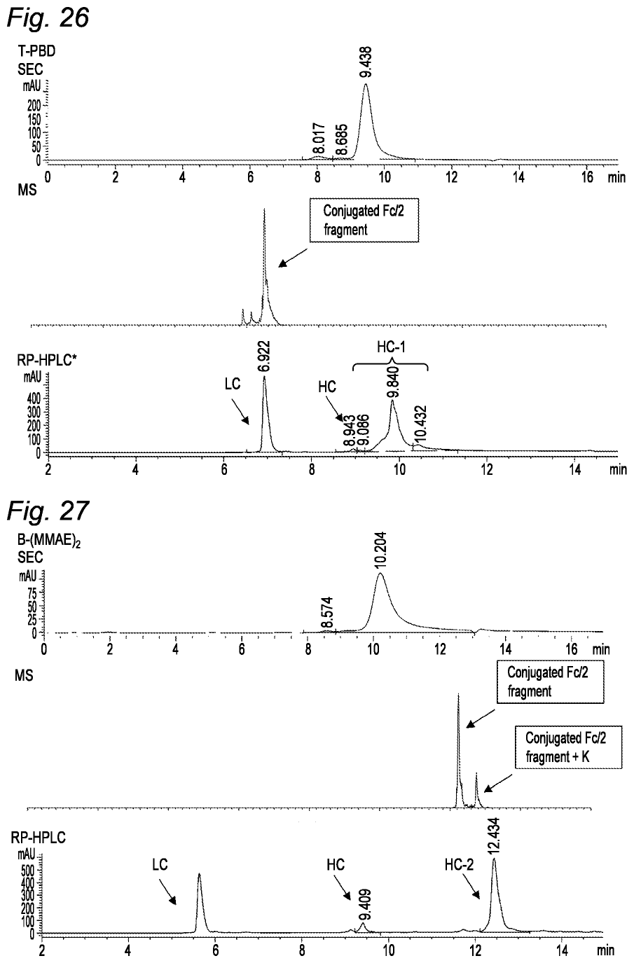

Deglycosylated trastuzumab (196 μL, 3 mg, 15.3 mg/mL in PBS 5.5) was incubated with BCN-HS-PEG₂-va-PABC-PBD 7 (40 μL, 5 mM in DMF) and mushroom tyrosinase (60 μL, 10 mg/mL in phosphate buffer pH 6.0, Sigma Aldrich T3824) for 16 h. Subsequently, the reaction was diluted with 300 μL PBS and centrifuged for 2 min at 13.000 rpm. The liquid was purified on a Superdex200 Increase 10/300 GL (GE Healthcare) column on an AKTA Purifier-10 (GE Healthcare). Mass spectral analysis of the IdeS-digested sample showed one major product (observed mass 25122 Da, approximately 90% of total Fc/2 fragment), corresponding to the conjugated Fc/2 fragment. SEC, MS and HPLC profiles of the conjugate depicted in FIG. 26.

### Example 27. Conjugation of deglycosylated B12 with BCN-HS-PEG₂-(vc-PABC-MMAE)₂ (6b)

Deglycosylated B12 (127 μL, 3 mg, 23.6 mg/mL in PBS 5.5) was incubated with BCN-HS-PEG₂-(vc-PABC-MMAE)₂ 6b (40 μL, 5 mM in DMF) and mushroom tyrosinase (60 μL, 10 mg/mL in phosphate buffer pH 6.0, Sigma Aldrich T3824) and PBS (73 μL, pH 5.5) for 16 h. Subsequently, the reaction was diluted with 300 μL PBS and centrifuged for 2 min at 13.000 rpm. The liquid was purified on a Superdex200 Increase 10/300 GL (GE Healthcare) column on an AKTA Purifier-10 (GE Healthcare). Mass spectral analysis of the IdeS-digested sample showed one major product (observed mass 26599 Da, approximately 70% of total Fc/2 fragment), corresponding to the conjugated Fc/2 fragment, and one minor product (observed mass 26687 Da, approximately 20% of total Fc/2 fragment), corresponding to the conjugated Fc/2 fragment with C-terminal lysine. SEC, MS and HPLC profiles of the conjugate of B12 with 6b depicted in FIG. 27.

SUMMARY TABLE

| Conjugate | % Conversion | % monomer | % Yield | DAR |
|---|---|---|---|---|
| T-6a (BCN-MMAE) | 90.0 | 98.2 | 88 | 1.8 |
| T-6b (BCN-MMAE)₂ | 91.6 | 97.5 | 50 | 3.67 |
| T-7 (BCN-PBD) | 97.0 | 94.5 | 84 | 3.88 |
| B12-6b (BCN-MMAE)₂ | 90.3 | 98.5 | 67 | 3.61 |

### Example 28. In Vitro Analysis

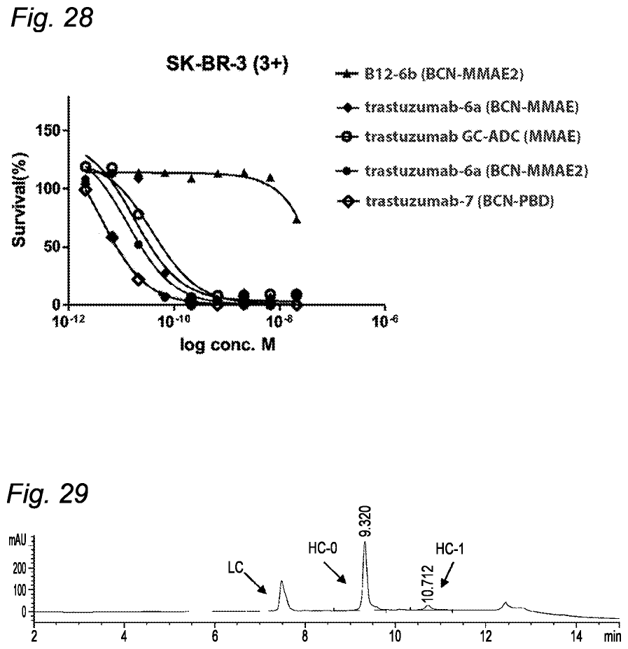

SK-BR-3 (Her2 3+) and MCF-7(Her2−) cells were plated in 96-well plates (5000 cells/well) in RPMI 1640 (Merck, R₇₃₈₈) supplemented with 10% fetal bovine serum (FBS) (ATCC® 30-2020™) and incubated overnight in a humidified atmosphere at 37° C. and 5% C₀₂. Compound T-6a/b, T-7 and B-6b were added in quadruplo in a three-fold dilution series to obtain a final concentration ranging from 2 μM to 21 nM. The cells were incubated for 5 days in a humidified atmosphere at 37° C. and 5% CO₂. The culture medium was replaced by 0.01 mg/mL resazurin (Sigma Aldrich) in RPMI 1640 (Merck, R7388) supplemented with 10% fetal bovine serum (FBS) (ATCC® 302020™). After approximately 3 to 4 hours in a humidified atmosphere at 37° C. and 5% CO₂ the fluorescence was detected with a fluorescence plate reader (Envision multipabel plate reader) at 531 nm excitation and 590 nm emission. The relative fluorescent units (RFU) were normalized to cell viability percentage by setting wells without cells at 0% viability and wells with untreated cells at 100% viability. Cell killing potential for the various constructs at different concentrations is plotted in FIG. 28.

### Example 29: Enzymatic Trimming of trastuzumab by Fusion Protein EndoSH

Trastuzumab (Herzuma) (1 mg, 10 mg/mL in PBS pH 7.4) was incubated with EndoSH (2 μL, 4.2 mg/mL) at 37° C. After overnight incubation the antibody was dialyzed (3 times to PBS pH 5.5) and concentrated to 11 mg/mL. Mass spectral analysis of a sample after IdeS treatment showed one major Fc/2 product (observed mass 24134 Da,) corresponding to the expected product.

### Example 30: Enzymatic Trimming of High-mannose trastuzumab by Fusion Protein EndoSH

Trastuzumab having high-mannose glycans (obtained via transient expression in CHO K1 cells in the presence of kifunensin performed by Evitria (Zurich, Switzerland) (1.4 mg, 11.4 mg/mL in PBS pH 7.4) was incubated with EndoSH (2.7 μL, 4.2 mg/mL) at 37° C. After incubation for 6 h the antibody was dialyzed (3 times to PBS pH 5.5) and concentrated to 16 mg/mL. Mass spectral analysis of a sample after IdeS treatment showed one major Fc/2 product (observed mass 23990 Da,) corresponding to the expected product.

### Example 31. Conjugation of Trimmed trastuzumab with BCN-HS-PEG₂-Vc-PABC-MMAE (6a)

Trimmed trastuzumab (20 μL, 0.2 mg, 10 mg/mL in PBS 5.5) was incubated with BCN-HS-PEG₂-vc-PABC-MMAE 6a (4 μL, 3.33 mM in DMF) and mushroom tyrosinase (4 μL, 10 mg/mL in phosphate buffer pH 6.0, Sigma Aldrich T3824) for 16 h. RP-HPLC analysis after DTT reduction showed about 10% conversion via a shift for the heavy chain peak corresponding to the conjugated product (FIG. 29).

### Example 32. Conjugation of Trimmed High-mannose trastuzumab with BCN-HS-PEG₂-vc-PABC-MMAE (6a)

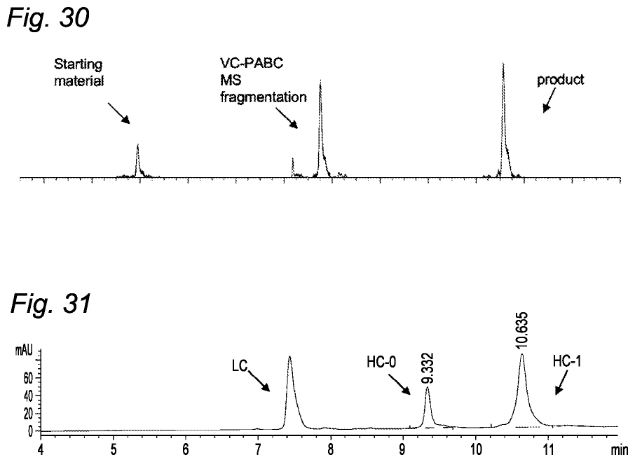

Trimmed high-mannose trastuzumab (20 μL, 0.2 mg, 10 mg/mL in PBS 5.5) was incubated with BCN-HS-PEG₂-vc-PABC-MMAE 6a (4 μL, 3.33 mM in DMF) and mushroom tyrosinase (4 μL, 10 mg/mL in phosphate buffer pH 6.0, Sigma Aldrich T3824) for 16 h. Subsequently, an extra portion of mushroom tyrosinase (4 μL, 10 mg/mL in phosphate buffer pH 6.0, Sigma Aldrich T3824) was added and the reaction was incubated for an additional 24 h. Mass spectral analysis of the IdeS-digested sample showed one major product (observed mass 25512 Da, approximately 40% of total Fc/2 fragment), and one fragmentation product (observed mass 24752 Da, approximately 30% of total Fc/2 fragment), both peaks correspond to the conjugated product (FIG. 30). RP-HPLC analysis (FIG. 31) indicates good conversion to the conjugate with a drug-to-antibody ratio of 1.50.

1. A process for preparing a glycoprotein-conjugate, comprising:
(a) providing an N-glycoprotein having an exposed tyrosine residue, wherein the exposed tyrosine residue is located within 10 amino acids of an N-glycosylation site, but the N-glycosylation site has been modified such that the glycoprotein does not contain a glycan longer than two monosaccharide residues within 10 amino acids of the exposed tyrosine residue;
(b) converting the phenol moiety of the exposed tyrosine residue into an ortho-quinone moiety by contacting the glycoprotein with an oxidative enzyme capable of oxidizing tyrosine;
(c) reacting the ortho-quinone moiety with an alkene or alkyne compound via a [4+2]cycloaddition, wherein the compound comprises a (hetero)cycloalkene or (hetero)cycloalkyne moiety and (i) a chemical handle to further modify the compound with a payload, or (ii) a payload.

2. The process according to claim 1, wherein the exposed tyrosine residue is located within 5 amino acids of the N-glycosylation site.

3. The process according to claim 1, wherein the N-glycoprotein having an exposed tyrosine residue is provided by:
(a1) subjecting an N-glycoprotein to deglycosylation by contacting it with an amidase to obtain an N-glycoprotein from which the glycan is removed; or
(a2) subjecting an N-glycoprotein to trimming by contacting it with an endoglycosidase, to form an N-glycoprotein having a glycan of structure -GlcNAc(Fuc)_{b}, wherein b is 0 or 1; or
(a3) providing a mutated N-glycoprotein wherein the glycosylated asparagine is replaced by a non-glycosylated amino acid.

4. The process according to claim 3, wherein the amidase is PNGase F.

5. The process according to claim 1, wherein the oxidative enzyme is tyrosinase or (poly)phenol oxidase.

6. The process according to claim 1, wherein (b) and (c) are performed in one-pot, by contacting the N-glycoprotein simultaneously with the oxidative enzyme and the alkene or alkyne compound.

7. The process according to claim 1, wherein the alkene or alkyne compound has a structure (3a) or (3b) 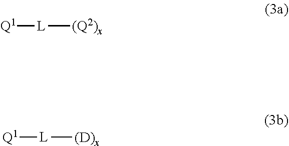
wherein:
Q¹ is a (hetero)cycloalkene or (hetero)cycloalkyne moiety;
L is a linker;
x is an integer in the range of 1-4;
Q² is a chemical handle that is reactive towards an appropriately functionalized payload but not towards Q¹;
D is a payload.

8. The process according to claim 1, wherein Q¹ is a (hetero)cycloalkyne according to structure (Q1): 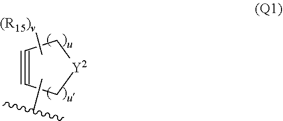
wherein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S⁽⁺⁾ is a cationic sulphur atom counterbalanced by B⁽⁻⁾, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=4, 5, 6, 7 or 8;
v=an integer in the range 8-16.

9. The process according to claim 8, wherein Q¹ is selected from the group consisting of (Q2)-(Q20): 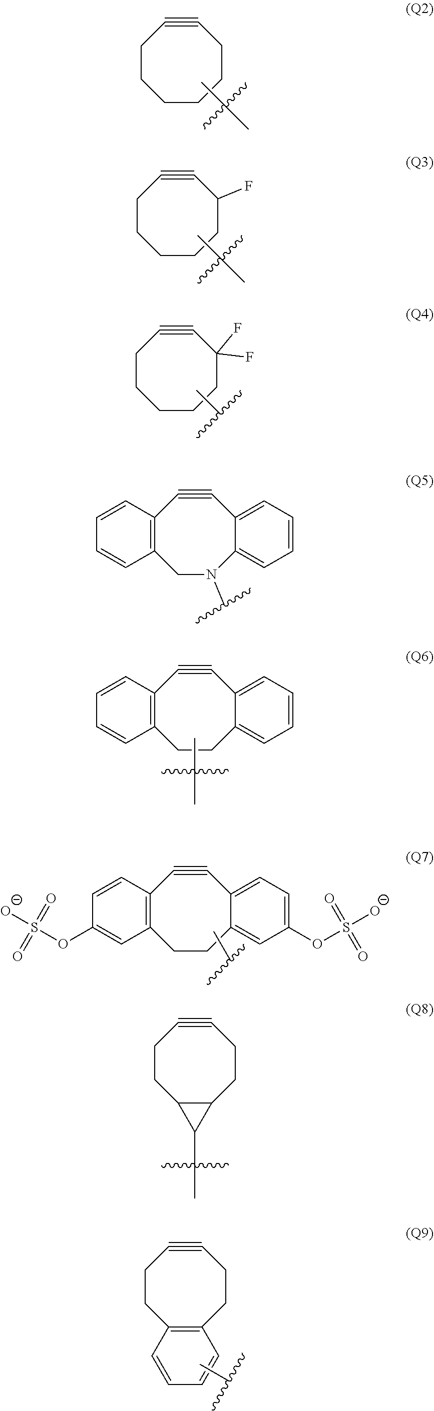 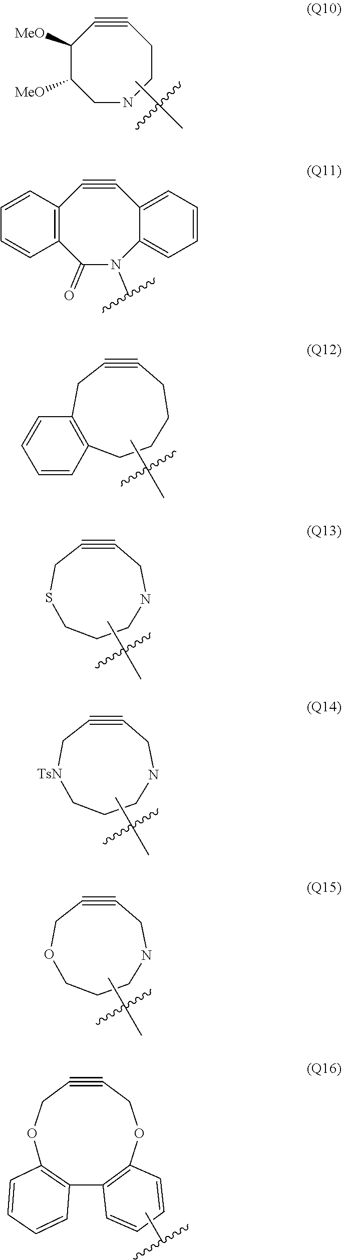 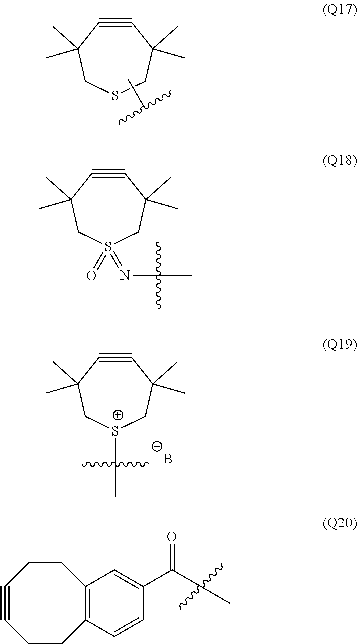
wherein B⁽⁻⁾ is an anion.

10. The process according to claim 9, wherein Q¹ is a cyclooctyne according to structure (Q42): 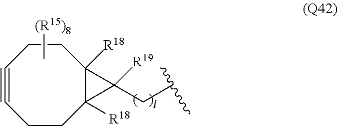
wherein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q¹ or D connected via a spacer moiety; and
I is an integer in the range 0 to 10;
or wherein Q¹ is a (hetero)cyclooctyne according to structure (Q43): 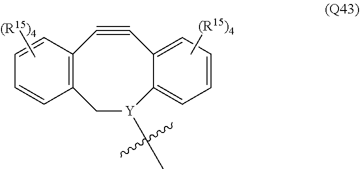
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵;
or wherein Q¹ is a heterocycloheptyne according to structure (Q37):

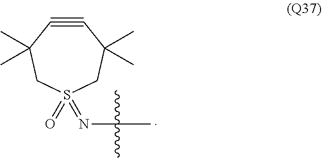

11. The process according to claim 1, wherein Q¹ is a (hetero)cycloalkene selected from the group consisting of, optionally substituted, (hetero)cyclopropenyl group, (hetero)cyclobutenyl group, a norbornene group, a norbornadiene group, trans-(hetero)cycloheptenyl group, trans-(hetero)cyclooctenyl group, trans-(hetero)cyclononenyl group or trans-(hetero)cyclodecenyl group.

12. The process according to claim 11, wherein Q¹ is selected from the group consisting of (Q44)-(Q56): 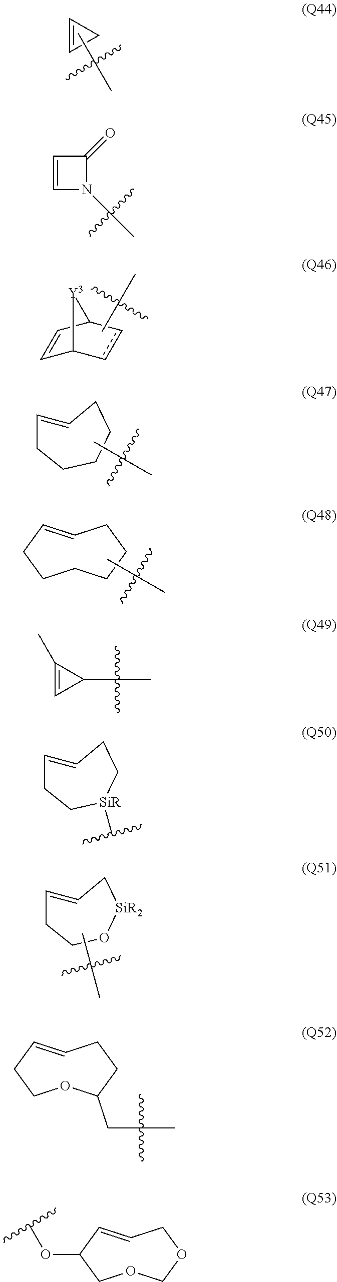 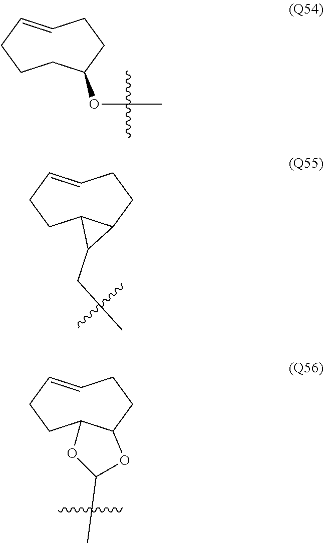
wherein Y³ is selected from C(R²⁴)₂, NR²⁴ or O, wherein each R²⁴ is individually hydrogen, C₁-C₆ alkyl or is connected to L, optionally via a spacer, and the bond labelled [symbol] is a single or double bond, and the R group(s) on Si in (Q50) and (Q51) is alkyl or aryl.

13. The process according to claim 1, wherein the compound comprises (i) a chemical handle to further modify the compound with a payload, and the process further comprises:
(d) subjecting the chemical handle, preferably Q², of the glycoprotein obtained in step (c) to a conjugation reaction with a payload having structure F²-D or F²-L²-(D)ₓ, wherein F² is reactive towards the chemical handle, L² is a linker and x is an integer in the range of 1-4.

14. The process according to claim 13, wherein the chemical handle is Q².

15. The process according to claim 1, wherein the payload D is selected from the group consisting of an active substance, a reporter molecule, a polymer, a solid surface, a hydrogel, a nanoparticle, a microparticle and a biomolecule.

16. A glycoprotein-conjugate according to structure (1a) or (1b): 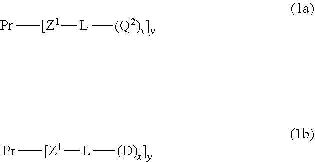
wherein:
Pr is an N-glycoprotein;
Z¹ comprises structure (Za) or (Zb): 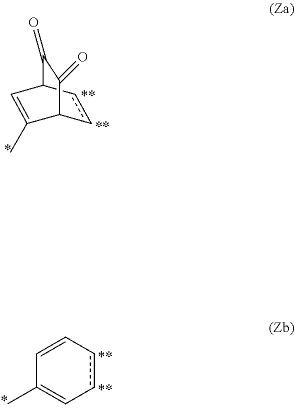
wherein the carbon labelled with * is directly connected to the peptide chain of the glycoprotein at an amino acid located within 10 amino acids of an N-glycosylation site, which has been modified such that the glycoprotein does not contain a glycan longer than two monosaccharide residues within 10 amino acids of the amino acid residue, and both of the carbon atoms labelled with ** are connected to L, and the bond depicted as [symbol] is a single bond or a double bond;
L is a linker;
x is an integer in the range of 1-4;
y is an integer in the range of 1-4;
Q² is a chemical handle that is reactive towards an appropriately functionalized payload;
D is a payload.

17. The glycoprotein-conjugate according to claim 16, wherein Z¹ has structure: 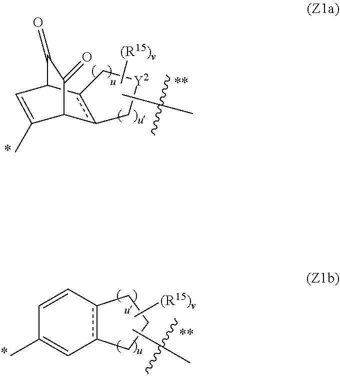
wherein:
the carbon labelled with * is directly connected to the peptide chain of the glycoprotein and the bond labelled with ** is connected to L, and the bond depicted as [symbol] is a single bond or a double bond;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S) is a cationic sulphur atom counterbalanced by B3, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 8-16.

18. The glycoprotein-conjugate according to claim 16, wherein Q² is reactive in a cycloaddition.

19. The glycoprotein-conjugate according to claim 16, wherein the payload D is selected from an active substance, a reporter molecule, a polymer, a solid surface, a hydrogel, a nanoparticle, a microparticle and a biomolecule.

20. A process for preparing a glycoprotein-conjugate, comprising reacting a glycoprotein according to structure (1a) according to claim 16, with a payload having structure D-F² or F²-L²-(D), wherein F² is reactive towards the chemical handle Q² in a conjugation reaction and wherein L² is a linker and x is an integer in the range of 1-4.

21. A pharmaceutical composition comprising the glycoprotein-conjugate according to structure (1b) according to claim 16 and a pharmaceutically acceptable carrier.

22. A method of treating cancer, comprising administering to a subject in need thereof a glycoprotein-conjugate according to structure (1b) according to claim 16.

23. A process for preparing a protein-conjugate, comprising:
(a) providing a mutant protein, which is in its native form unreactive towards oxidative enzymes capable of oxidizing tyrosine, but is rendered reactive towards such enzymes by providing a mutated form of the protein, wherein a tyrosine residue is introduced at a non-native position of the amino acid sequence of the protein where it is reactive towards oxidative enzymes capable of oxidizing tyrosine;
(b) converting the phenol moiety of the tyrosine residue into an ortho-quinone moiety by contacting the protein with an oxidative enzyme capable of oxidizing tyrosine;
(c) reacting the ortho-quinone moiety with an alkene or alkyne compound via a [4+2]cycloaddition, wherein the compound comprises a (hetero)cycloalkene or (hetero)cycloalkyne moiety and (i) a chemical handle to further modify the compound with a payload, or (ii) a payload.

24. A protein-conjugate according to structure (1a) or (1b): 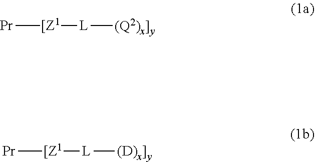
wherein:
Pr is a protein;
Z¹ comprises structure (Za) or (Zb): 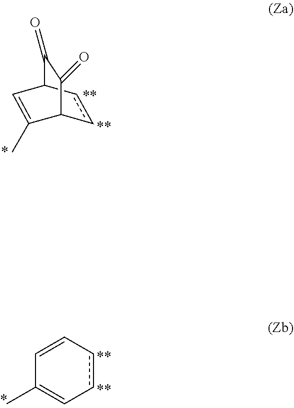
wherein the carbon labelled with * is directly connected to the peptide chain of the glycoprotein at an amino acid which is in the native form of the protein not a tyrosine residue, and both of the carbon atoms labelled with ** are connected to L, and the bond depicted as [symbol] is a single bond or a double bond;
L is a linker;
x is an integer in the range of 1-4;
y is an integer in the range of 1-4;
Q² is a chemical handle that is reactive towards an appropriately functionalized payload;
D is a payload.

25. The protein-conjugate according to claim 24, wherein the amino acid to which the connecting group Z¹ is connected is located at a position where a tyrosine residue is reactive towards oxidative enzymes capable of oxidizing tyrosine.

26. The protein-conjugate according to claim 24, wherein Pr is a mutant protein which is in its native form unreactive towards oxidative enzymes capable of oxidizing tyrosine, but is rendered reactive towards such enzyme by providing a mutated form of the protein, wherein a tyrosine residue is introduced at a non-native position in a position of the amino acid sequence of the protein where it is reactive towards oxidative enzymes capable of oxidizing tyrosine.
